(19) United States
(12) Patent Application Publication
(10) Pub. No.: US 2022/0332764 A1
(43) Pub. Date: Oct. 20, 2022

(54) T CELL EPITOPES OF HCMV AND USES OF THEREOF

(71) Applicants: EBERHARD KARLS UNIVERSITÄT TÜBINGEN, TÜBINGEN (DE); ALBERT-LUDWIGS-UNIVERSITÄT FREIBURG, FREIBURG (DE)

(72) Inventors: ANNIKA NELDE, TÜBINGEN (DE); MAREN LÜBKE, REUTLINGEN (DE); STEFANIE SPALT, BISSINGEN AN DER TECK (DE); DANIEL KOWALEWSKI, KIRCHENTELLINSFURT (DE); HANS-GEORG RAMMENSEE, TÜBINGEN (DE); ANNE HALENIUS, FREIBURG (DE); COSIMA ZIMMERMANN, BERLIN (DE); HARTMUT HENGEL, GUNDELFINGEN (DE); LIANE BAUERSFELD, LÖRRACH (DE); VU THUY KHANH LE-TRILLING, ESSEN (DE)

(21) Appl. No.: 17/638,833

(22) PCT Filed: Aug. 28, 2020
(86) PCT No.: PCT/EP2020/074114

§ 371 Date: Feb. 27, 2022

(30) Foreign Application Priority Data
Aug. 29, 2019 (DE) 10 2019 123 229.5

(51) Int. Cl. C07K 14/005; C07K 16/08; C07K 14/725; C12N 5/0783; A61K 39/25

(52) U.S. Cl. CPC C07K 14/005; C07K 16/088; C07K 14/7051; C12N 5/0638; A61K 39/25; C12N 2710/16122; C12N 2710/16134

(57) ABSTRACT

The present invention relates to relates to T cell epitope peptides, proteins, nucleic acids and cells for use in immunother-apeutic methods. In particular, the present invention relates to the immunotherapy of viral infection. The present invention specifically relates to virus-associated T-cell peptide epitopes, alone or in combination with other virus-associated peptides that can serve as active pharmaceutical ingredients of vaccine compositions that stimulate anti-viral immune responses, or to stimulate T cells ex vivo and transfer into patients. Peptides bound to molecules of the major histocompatibility complex (MHC), or peptides as such, can also be targets of antibodies, soluble T-cell receptors, and other binding molecules.

The present invention relates to relates to T cell epitope peptides, proteins, nucleic acids and cells for use in immunotherapeutic methods. In particular, the present invention relates to the immunotherapy of viral infection. The present invention specifically relates to virus-associated T-cell peptide epitopes, alone or in combination with other virus-associated peptides that can serve as active pharmaceutical ingredients of vaccine compositions that stimulate anti-viral immune responses, or to stimulate T cells ex vivo and transfer into patients. Peptides bound to molecules of the major histocompatibility complex (MHC), or peptides as such, can also be targets of antibodies, soluble T-cell receptors, and other binding molecules.

### BACKGROUND OF THE INVENTION

In healthy individuals, immune control of persistent human cytomegalovirus (HCMV) infection is effectively mediated by virus-specific CD4+ and CD8+ T-cells. However, identification of the repertoire of T-cell specificities for HCMV is hampered by the immense protein coding capacity of this betaherpes virus.

HCMV-associated pathologies are a common cause of post-transplant morbidity and mortality. Identification of the physiological targets of anti-HCMV T-cell responses will allow for improvements of current treatments of these complications, e.g. by adoptive T-cell transfer. Although HCMV-derived T-cell epitopes could already be identified, some of these are recognized infrequently. Furthermore, research has mainly focused on subsets of HLA alleles, leaving gaps in the HLA coverage.

Primary infection with human cytomegalovirus (HCMV) is followed by lifelong latency with recurrent cycles of endogenous reactivation. The prevalence of HCMV in adults ranges from 40 to 90% and increases with age (1, 2).

While in the immunocompetent host infection is usually controlled by an HCMV-specific immune response, severe damage is common in immunologically restricted populations. Congenital infection of the fetus has considerable consequences, involving the central nervous system with sensorineural hearing loss, mental retardation or even death. HCMV infection or reactivation is a major cause of morbidity and mortality in immunocompromised individuals such as AIDS patients or transplant recipients, since CD8+ but also CD4+ T-cell immunity plays a critical role in preventing lethal infection (3-5). Further, it is thought that subclinical infections with HCMV are involved in a variety of diseases, for example certain cancers, inflammatory, hypertensive, and pulmonary diseases (6-10).

Current HCMV treatments include antiviral drugs and attempts to exploit the humoral and cellular immune responses. Furthermore, considerable effort is made on the development of an HCMV vaccine. For all immunological therapies deeper insights into potential target structures are vitally important. Since with its almost 236 kbp long double-stranded DNA genome, HCMV has the largest genome among human herpesviruses, the majority of studies on cytotoxic T-lymphocyte responses have so far been restricted to a very limited selection of HCMV antigens; most prominent among them are the immunodominant antigens pp65 and IE1 (11-15).

A number of studies have clearly demonstrated that the HCMV-specific T-cell response targets a much broader spectrum of HCMV antigens (16-18). To date, the identification of most HCMV-specific T-cell targets has been based on prediction methods (16, 19, 20) or the use of overlapping peptides (18). The approach of direct isolation of viral ligands from infected target cells, successfully used for some viral infections (21-24), has been cumbersome due to strict control of peptide presentation by HCMV encoded HLA class I (HLA-I) immunoevasins (25-30).

Glycoproteins encoded by the US6 gene family are able to impair the stability and localization of HLA-I. The glycoproteins US2 and US11 bind HLA-I and mediate their reverse transport into the cytosol for subsequent degradation by the proteasome (31-33). US6 prevents the assembly of the HLA-I/peptide complexes by inhibiting the transport of peptides into the endoplasmic reticulum by the transporter associated with antigen processing (TAP) (26, 34). The product of US3 forms complexes with assembled J32-microglobulin-associated HLA-I heavy chains, thereby blocking maturation and translocation of HLA-I molecules to the cell surface (25).

Therefore, development of better therapies and prevention strategies in HCMV is of considerable importance. Other objects of the present invention will become apparent to the person of skill when studying the following more detailed description of the invention.

In a first aspect of the present invention, the present invention relates to a peptide comprising an amino acid sequence selected from the group consisting of SEQ ID NO: 1 to SEQ ID NO: 101, and variant sequences of SEQ ID NO: 1 to SEQ ID NO: 101 that comprise one amino acid exchange and bind to molecule(s) of the major histocompatibility complex (MHC) and/or induce T cells cross-reacting with said variant peptide, and a pharmaceutical acceptable salts thereof, wherein said peptide has an overall length of between 8 and 30, preferably 9 and 30, amino acids.

Preferred is a peptide or variant according to the present invention, wherein said peptide consists or consists essentially of an amino acid sequence according to any of SEQ ID NO: 1 to SEQ ID NO: 100 or optionally comprises an extension of one N- and/or C-terminal, preferably naturally occurring, amino acid.

More preferred is a peptide or variant according to the present invention, wherein the amino acid sequence is selected from SEQ ID NO: 1 to 4, 24 to 29, 40, 41, 51 to 55, 67, 68, 80, 87 to 89, and 99 to 101.

Here, the inventors present a novel approach which employs HCMV deletion mutant viruses, lacking HLA class I immunoevasins, to allow direct identification of naturally presented HCMV-derived HLA ligands by mass spectrometry. The use of varying HCMV deletion mutants resulted in a higher variability of identified HCMV-derived peptide species, and demonstrated that HLA-I immunoevasins affect not only the quantity, but also the quality of HLA-I antigen processing and presentation.

The present invention thus further relates to a method for identifying HCMV-derived HLA class I ligands comprising generating a HCMV deletion mutant virus, lacking at least one functional HLA class I immunoevasin, preferably all HLA class I immunoevasins, infecting a cell culture, for example a fibroblast cell culture, expressing at least one HLA-I type of interest, and isolating and identifying presented HCMV-derived HLA-I ligands from said cell culture. The ligands (peptides) can then be used in the context of the present invention, i.e. for respective vaccines, therapies, and the generation of T-cells and/or -receptors as described herein. Preferred is a peptide or variant according to the present invention, wherein said peptide consists or consists essentially of an amino acid sequence according to any of SEQ ID NO: 1 to SEQ ID NO: 100 or optionally comprises an extension of one N- and/or C-terminal, preferably naturally occurring, amino acid. More preferred is a peptide or variant according to the present invention, wherein the amino acid sequence is selected from SEQ ID NO: 1 to 4, 24 to 29, 40, 41, 51 to 55, 67, 68, 80, 87 to 89, and 99 to 101.

Using this strategy, the inventors identified 368 unique HCMV-derived HLA class I ligands representing an unexpectedly broad panel of 123 HCMV antigens. Functional characterization revealed memory T-cell responses in seropositive individuals for a substantial proportion (28%) of these novel peptides. Importantly, the inventors frequently detected multiple HCMV-directed specificities in the memory T-cell pool of single individuals, indicating that physiological anti-HCMV T-cell responses are directed against a broad range of antigens. Furthermore, these memory T cells were multifunctional (IFNy, TNF) and able to exert cytolytic activity in vitro.

Thus, the unbiased identification of naturally presented viral epitopes enabled a comprehensive and systematic assessment of the physiological repertoire of anti-HCMV T-cell specificities in seropositive individuals. This approach proved to be superior to procedures applying in silico analysis to identify true viral antigens, and the use of varying HCMV deletion mutants resulted in a higher variability of identified HCMV-derived peptide species.

The following tables show the peptides according to the present invention, and their respective SEQ ID NOs.

TABLE 1
Peptide epitopes of the invention,
underlying protein, sequence, actual
HLA restriction (where determined).

| | | Actual HLA |
|---|---|---|
| | | restriction |
| | | (identified |
| Source | | using |
| protein | | tetramer |
| and | Sequence/ | staining |
| position | SEQ ID NO: | and ICS) |
| US8 74-82 | GVLDAVWRV | A*02:01 |
| | (SEQ ID NO: 1) | |
| UL150A 152-161 | ALWDVALLEV | A*02:01 |
| | (SEQ ID NO: 2) | |
| UL100 200-208 | TLIVNLVEV | A*02:01 |
| | (SEQ ID NO: 3) | |
| UL44 259-267 | GLFA VENFL | DR |
| | (SEQ ID NO: 4) | |
| UL71 40-48 | FLDENFKQL | DR |
| | (SEQ ID NO: 5) | |
| UL105 431-439 | RLFDLPVYC | |
| | (SEQ ID NO: 6) | |
| UL29 175-183 | RLQPNVPLV | |
| | (SEQ ID NO: 7) | |
| US16 134-144 | GLLAHIPALGV | |
| | (SEQ ID NO: 8) | |
| US29 293-301 | ALSPSTSKV | |
| | (SEQ ID NO: 9) | |
| UL29 344-352 | SLYEANPEL | |
| | (SEQ ID NO: 10) | |
| UL86 146-154 | TILDKILNV | |
| | (SEQ ID NO: 11) | |
| US16 186-194 | TLINGVWVV | |
| | (SEQ ID NO: 12) | |
| US16 134-142 | GLLAHIPAL | |
| | (SEQ ID NO: 13) | |
| UL48 132-141 | ALYPEYIYTV | |
| | (SEQ ID NO: 14) | |
| UL47 766-744 | GLNERLLSV | |
| | (SEQ ID NO: 15) | |
| UL34 130-138 | ALFNQLVFTA | |
| | (SEQ ID NO: 16) | |
| UL56 124-132 | FTDNVRFSV | |
| | (SEQ ID NO: 17) | |
| UL128 145-153 | GLDQYLESV | |
| | (SEQ ID NO: 18) | |
| UL84 133-141 | ALLGRLYFI | |
| | (SEQ ID NO: 19) | |
| UL4 96-104 | NYNEQHYRY | |
| | (SEQ ID NO: 20) | |
| US27 276-284 | LYVGQFLAY | |
| | (SEQ ID NO: 21) | |
| UL4 88-97 | SFFPKLQGNY | |
| | (SEQ ID NO: 22) | |
| UL4 89-97 | FFPKLQGNY | |
| | (SEQ ID NO: 23) | |
| UL16 162-170 | YPRPPGSGL | B*07:02 |
| | (SEQ ID NO: 24) | |
| UL83 417-426 | TPRVTGGGAM | B*07:02 |
| | (SEQ ID NO: 25) | |
| TRS1 166-174 | SPRDAWIVL | B*07:02 |
| | (SEQ ID NO: 26) | |
| UL52 349-357 | SPSRDRFVQL | B*07:02 |
| | (SEQ ID NO: 27) | |
| UL23 22-30 | RPWKPGQRV | B*07:02 |
| | (SEQ ID NO: 28) | |
| UL46 76-84 | SPRHLYISL | B*07:02 |
| | (SEQ ID NO: 29) | |
| UL38 225-235 | IPMTFVDRDSL | |
| | (SEQ ID NO: 30) | |
| US30 313-321 | RPFPSTHQL | |
| | (SEQ ID NO: 31) | |
| UL83 49-57 | RVSQPSLIL | |
| | (SEQ ID NO: 32) | |
| UL82 245-254 | SPHPPTSVFL | |
| | (SEQ ID NO: 33) | |
| UL27 485-493 | IPDYRSVSL | |
| | (SEQ ID NO: 34) | |
| UL31 310-317 | APFGRVSV | |
| | (SEQ ID NO: 35) | |
| TRS1/IRS1 | IPVERQAL | |
| 92-99 | (SEQ ID NO: 36) | |
| UL98 135-143 | APNYRQVEL | |
| | (SEQ ID NO: 37) | |
| UL40 210-218 | LPNDHHYAL | |
| | (SEQ ID NO: 38) | |
| US 12 82-89 | APYLRDTL | |
| | (SEQ ID NO: 39) | |
| UL112/UL113 | SENGNLQVTY | B*44:02 |
| 125-134 | (SEQ ID NO: 40) | |
| UL117 358-366 | HETGVYQMW | B*44:02 |
| | (SEQ ID NO: 41) | |
| UL17 24-32 | DEQVSKRSW | B*44:02 |
| | (SEQ ID NO: 42) | |
| TRL12 402-410 | SESEFIVRY | B*44:02 |
| | (SEQ ID NO: 43) | |
| UL147A 51-59 | EEQDYRALL | |
| | (SEQ ID NO: 44) | |
| UL78 150-158 | RENAGVALY | |
| | (SEQ ID NO: 45) | |
| US21 71-80 | AEPNFPKNVW | |
| | (SEQ ID NO: 46) | |
| TRS1/IRS1 | EEATALGREL | |
| 276-285 | (SEQ ID NO: 47) | |
| US11 103-111 | SESLVAKRY | |
| | (SEQ ID NO: 48) | |
| UL54 755-763 | LENGVTHRF | |
| | (SEQ ID NO: 49) | |
| US22 72-81 | REQAAIPQIY | |
| | (SEQ ID NO: 50) | |
| UL105 715-723 | YADPFFLKY | A*01:01 |
| | (SEQ ID NO: 51) | |
| UL44 245-253 | VTEHDTLLY | A*01:01 |
| | (SEQ ID NO: 52) | |
| UL69 569-578 | RTDPATLTAY | A*01:01 |
| | (SEQ ID NO: 53) | |
| US28 122-130 | ITEIALDRY | A*01:01 |
| | (SEQ ID NO: 54) | |
| UL55 657-665 | NTDFRVLELY | A*01:01 |
| | (SEQ ID NO: 55) | |
| UL36 82-91 | FVEGPGFMRY | |
| | (SEQ ID NO: 56) | |
| UL148 282-290 | SLDRFIVQY | DR |
| | (SEQ ID NO: 57) | |
| UL25 370-379 | YTSRGALYLY | |
| | (SEQ ID NO: 58) | |
| UL86 1346-1354 | TSETHFGNY | |
| | (SEQ ID NO: 59) | |
| US34 92-101 | GSDALPAGLY | |
| | (SEQ ID NO: 60) | |
| UL48 1607-1617 | VTDYGNVAFKY | |
| | (SEQ ID NO: 61) | |
| IRS1/TRS1 | LLDELGAVFGY | |
| 464-474 | (SEQ ID NO: 62) | |
| UL112/UL113 | ISENGNLQVTY | |
| 124-134 | (SEQ ID NO: 63) | |
| UL105 616-624 | VTDPEHLMM | |
| | (SEQ ID NO: 64) | |
| UL105 360-369 | DLDFGDLLKY | |
| | (SEQ ID NO: 65) | |
| UL78 222-232 | YSDRRDHVWSY | |
| | (SEQ ID NO: 66) | |
| UL77 228-236 | GLYTQPRWK | A*03:01 |
| | (SEQ ID NO: 67) | |
| UL57 790-798 | RVKNRPIYR | A*03:01 |
| | (SEQ ID NO: 68) | |
| UL36 51-60 | RSALGPFVGK | A*03:01 |
| | (SEQ ID NO: 69) | |
| UL123 184-192 | KLGGALQAK | |
| | (SEQ ID NO: 70) | |
| US33A 13-21 | KLGYRPHAK | A*03:01 |
| | (SEQ ID NO: 71) | |
| US24 136-145 | RVYAYDTREK | |
| | (SEQ ID NO: 72) | |
| UL25 580-588 | GVSSVTLLK | |
| | (SEQ ID NO: 73) | |
| UL84 3-11 | RVDPNLRNR | |
| | (SEQ ID NO: 74) | |
| UL70 698-706 | SVRLPYMYK | |
| | (SEQ ID NO: 75) | |
| UL79 237-245 | RTFAGTLSR | |
| | (SEQ ID NO: 76) | |
| UL57 1044-1052 | RLADVLIKR | |
| | (SEQ ID NO: 77) | |
| UL70 697-706 | RSVRLPYMYK | |
| | (SEQ ID NO: 78) | |
| UL122 113-121 | SVSSAPLNK | |
| | (SEQ ID NO: 79) | |
| UL13 465-473 | YLVRRPMTI | B*08:01 |
| | (SEQ ID NO: 80) | |
| UL36 199-207 | VMKFKETSF | |
| | (SEQ ID NO: 81) | |
| UL84 239-247 | TPLLKRLPL | |
| | (SEQ ID NO: 82) | |
| UL40 170-178 | HLKLRPATF | |
| | (SEQ ID NO: 83) | |
| UL84 500-507 | FISSKHTL | |
| | (SEQ ID NO: 84) | |
| UL44 26-34 | QLRS VIRAL | |
| | (SEQ ID NO: 85) | |
| UL148 1-8 | MLRLLFTL | B*08:01 |
| | (SEQ ID NO: 86) | |
| UL83 116-123 | LPLKMLNI | B*51:01 |
| | (SEQ ID NO: 87) | |
| UL38 156-164 | FPVEVRSHV | B*51:01 |
| | (SEQ ID NO: 88) | |
| UL56 503-511 | DARSRIHNV | B*51:01 |
| | (SEQ ID NO: 89) | |
| UL71 330-338 | IPPPQIPFV | |
| | (SEQ ID NO: 90) | |
| US28 158-166 | IAIPHFMVV | |
| | (SEQ ID NO: 91) | |
| US23 65-73 | IPHNWFLQV | |
| | (SEQ ID NO: 92) | |
| UL33 162-170 | VPAAVYTTV | |
| | (SEQ ID NO: 93) | |
| UL14 66-74 | FPAHDWPEV | |
| | (SEQ ID NO: 94) | |
| UL122 449-457 | MPVTHPPEV | |
| | (SEQ ID NO: 95) | |
| UL75 540-549 | FPDATVPATV | |
| | (SEQ ID NO: 96) | |
| UL48 1322-1331 | LPYLSAERTV | |
| | (SEQ ID NO: 97) | |
| UL147A2-10 | SLFYRAVAL | |
| | (SEQ ID NO: 98) | |
| UL26 61-69 | LPYPRGYTL | B*08:01/B*51:01 |
| | (SEQ ID NO: 99) | B*08:01/B*51:01 |
| UL34 180-188 | LPHERHREL | B*08:01 |
| | (SEQ ID NO: 100) | |

TABLE 2
Preferred dominant epitopes of the invention,
sequence, actual HLA restriction

| | Sequence | actual HLA |
|---|---|---|
| Protein | SEQ ID NO: | restriction |
| US8 74-82 | GVLDAVWRV | A*02:01 |
| | SEQ ID NO: 1 | |
| UL150A 152- | ALWDVALLEV | A*02:01 |
| 161 | SEQ ID NO: 2 | |
| UL100 200-208 | TLIVNLVEV | A*02:01 |
| | SEQ ID NO: 3 | |
| UL44 259-267 | GLFAVENFL | Class II |
| | SEQ ID NO: 4 | |
| UL16 162-170 | YPRPPGSGL* | B*07:02 |
| | SEQ ID NO: 24 | |
| UL83 417-426 | TPRVTGGGAM* | B*07:02 |
| | SEQ ID NO: 25 | |
| TRS1 166-174 | SPRDAWIVL | B*07:02 |
| | SEQ ID NO: 26 | |
| UL52 349-357 | SPSRDRFVQL | B*07:02 |
| | SEQ ID NO: 27 | |
| UL23 22-30 | RPWKPGQRV | B*07:02 |
| | SEQ ID NO: 28 | |
| UL46 76-84 | SPRHLYISL | B*07:02 |
| | SEQ ID NO: 29 | |
| UL112/UL113 | SENGNLQVTY | B*44:02 |
| 125-134 | SEQ ID NO: 40 | |
| UL117 358-366 | HETGVYQMW | B*44:02 |
| | SEQ ID NO: 41 | |
| UL105 715-723 | YADPFFLKY* | A*01:01 |
| | SEQ ID NO: 51 | |
| UL44 245-253 | VTEHDTLLY* | A*01:01 |
| | SEQ ID NO: 52 | |
| UL69 569-578 | RTDPATLTAY | A*01:01 |
| | SEQ ID NO: 53 | |
| US28 122-130 | ITEIALDRY | A*01:01 |
| | SEQ ID NO: 54 | |
| UL55 657-665 | NTDFRVLELY | A*01:01 |
| | SEQ ID NO: 55 | |
| UL77 228-236 | GLYTQPRWK | A*03:01 |
| | SEQ ID NO: 67 | |
| UL57 790-798 | RVKNRPIYR | A*03:01 |
| | SEQ ID NO: 68 | |
| UL34 180-188 | LPHERHREL | B*08:01 |
| | SEQ ID NO: 100 | |
| UL26 61-69 | LPYPRGYTL | B*08:01/51:01 |
| | SEQ ID NO: 99 | |
| UL13 465-473 | YLVRRPMTI | B*08:01 |
| | SEQ ID NO: 80 | |
| UL83 116-123 | LPLKMLNI* | B*51:01 |
| | SEQ ID NO: 87 | |
| UL38 156-164 | FPVEVRSHV | B*51:01 |
| | SEQ ID NO: 88 | |
| UL26 61-69 | LPYPRGYTL | B*08:01/51:01 |
| | SEQ ID NO: 99 | |
| UL56 503-511 | DARSRIHNV | B*51:01 |
| | SEQ ID NO: 89 | |
| UL83 495-503 | NLVPMVATV* | A*02:01 |
| | SEQ ID NO: 101 | |

The present invention further relates to a peptide or variant thereof according to the present invention, wherein said peptide is modified and/or includes non-peptide bonds.

The present invention further relates to a peptide or variant thereof according to the present invention, wherein said peptide is part of a fusion protein, in particular comprising the N-terminal amino acids of the HLA-DR antigen-associated invariant chain (li).

The present invention further relates to an antibody, in particular a soluble or membrane-bound antibody that specifically binds to the peptide or variant thereof according to the present invention, preferably the peptide or variant thereof according to the present invention when bound to an MHC molecule.

The present invention further relates to a T cell receptor, preferably a recombinant, soluble or membrane-bound T cell receptor that is reactive with an HLA ligand, wherein said ligand is at least 75% identical, preferably at least 88% identical, and most preferred 100% identical to an amino acid sequence according to the present invention.

The present invention further relates to a T cell receptor according to the present invention, wherein said T cell receptor is provided as a soluble molecule, and optionally comprises an effector function, such as an immune stimulating domain or toxin.

The present invention further relates to a nucleic acid, encoding a peptide or variant thereof according to the present invention, the antibody according to the present invention or the T cell receptor according to the present invention, wherein said nucleic acid is optionally linked to a heterologous promoter sequence.

The present invention further relates to an expression vector expressing the nucleic acid according to the present invention.

The present invention further relates to a recombinant host cell comprising a recombinant peptide according to the present invention, a recombinant antibody according to the present invention, a recombinant T cell receptor according to the present invention, the nucleic acid according to claim 7 or the expression vector according to the present invention, wherein said host cell preferably is an antigen presenting cell such as a dendritic cell, or preferably is a T cell or NK cell.

The present invention further relates to a method for producing the peptide or variant thereof according to the present invention, the antibody according to the present invention, or the T cell receptor according to the present invention, the method comprising culturing the host cell according to the present invention that presents the peptide according to the present invention, or expresses the nucleic acid according to the present invention or comprises the expression vector according to the present invention, and isolating said peptide or variant thereof, said antibody or said T cell receptor from said host cell and/or its culture medium.

The present invention further relates to an in vitro method for producing activated T lymphocytes, the method comprising contacting in vitro T cells with antigen loaded human class I or II MHC molecules expressed on the surface of a suitable antigen-presenting cell or an artificial construct mimicking an antigen-presenting cell for a period of time sufficient to activate said T cells in an antigen specific manner, wherein said antigen is a peptide according to the present invention.

The present invention further relates to an activated T lymphocyte, produced by the method according to the present invention that selectively recognizes a cell which presents a polypeptide comprising an amino acid sequence as disclosed herein.

The present invention further relates to a pharmaceutical composition comprising at least one active ingredient selected from the group consisting of the peptide or variant thereof according to the present invention, the antibody according to the present invention, the T cell receptor according to the present invention, the nucleic acid according to the present invention, the expression vector according to the present invention, the host cell according to the present invention or the activated T lymphocyte according to the present invention, and a pharmaceutically acceptable carrier, and optionally additional pharmaceutically acceptable excipients and/or stabilizers.

The present invention further relates to a method for producing a personalized anti-viral vaccine, said method comprising: a) identifying at least one HCMV-associated peptide according to any one of SEQ ID NO: 1 to SEQ ID NO: 101 in a sample from said individual patient; b) selecting at least one peptide as identified in said sample from step a), and c) formulating the at least one peptide as selected in step b) into a personalized anti-viral vaccine.

The present invention further relates to a peptide or variant thereof according to the present invention, the antibody according to the present invention, the T cell receptor according to the present invention, the nucleic acid according to the present invention, the expression vector according to the present invention, the host cell according to the present invention or the activated T lymphocyte according to the present invention, the pharmaceutical composition according to the present invention, or the vaccine as produced according to the present invention for use in medicine.

The present invention further relates to a peptide or variant thereof according to the present invention, the antibody according to the present invention, the T cell receptor according to the present invention, the nucleic acid according to the present invention, the expression vector according to the present invention, the host cell according to the present invention or the activated T lymphocyte according to the present invention, the pharmaceutical composition according to the present invention, or the vaccine as produced according to the present invention for use in the diagnosis and/or treatment of HCMV infection, or for use in the manufacture of a medicament against HCMV infection.

The present invention further relates to a peptide or variant thereof according to the present invention, the antibody according to the present invention, the T cell receptor according to the present invention, the nucleic acid according to the present invention, the expression vector according to the present invention, the host cell according to the present invention or the activated T lymphocyte according to the present invention, the pharmaceutical composition according to the present invention, or the vaccine as produced according to the present invention for use according to the present invention, wherein said HCMV infection exhibits a co-morbidity with cancer, inflammatory diseases, hypertensive diseases, and pulmonary diseases.

The present invention further relates to a kit comprising: a) a container comprising a peptide or variant thereof according to the present invention, the antibody according to the present invention, the T cell receptor according to the present invention, the nucleic acid according to the present invention, the expression vector according to the present invention, the host cell according to the present invention, or the activated T lymphocyte according to the present invention, the pharmaceutical composition according to the present invention, or the vaccine as produced according to the present invention, in solution or in lyophilized form; b) optionally, a second container containing a diluent or reconstituting solution for the lyophilized formulation; c) optionally, at least one additional peptide selected from the group consisting of SEQ ID NO: 1 to SEQ ID NO: 101, and d) optionally, instructions for (i) use of the solution or (ii) reconstitution and/or use of the lyophilized formulation, and e) a substance or combination of substances acting as an adjuvant, i.e. acting as an inducer of immune responses Preferred is the kit according to the present invention, further comprising one or more of (iii) a buffer, (iv) a diluent, (v) a filter, (vi) a needle, or (v) a syringe, or vi) a mixing device.

Finally, the present invention further relates to a method for treating HCMV infection in target cells in a patient, wherein said target cells present at least one peptide comprising an amino acid sequence according to the present invention, comprising administering to said patient an effective amount of activated T lymphocytes according to the present invention, the pharmaceutical composition according to the present invention, and/or of the vaccine as produced according to the present invention.

There are two classes of MHC-molecules, MHC class I and MHC class II. MHC class I molecules are composed of an alpha heavy chain and beta-2-microglobulin, MHC class II molecules of an alpha and a beta chain. Their three-dimensional conformation results in a binding groove, which is used for non-covalent interaction with peptides.

MHC class I molecules can be found on most nucleated cells. They present peptides that result from proteolytic cleavage of predominantly endogenous proteins, defective ribosomal products (DRIPs) and larger peptides. However, peptides derived from endosomal compartments or exogenous sources are also frequently found on MHC class I molecules. This non-classical way of class I presentation is referred to as cross-presentation in literature (Brossart and Bevan, 1997; Rock et al., 1990). MHC class II molecules can be found predominantly on professional antigen presenting cells (APCs), and primarily present peptides of exogenous or transmembrane proteins that are taken up by APCs e.g. during endocytosis, and are subsequently processed.

Complexes of peptide and MHC class I are recognized by CD8-positive T cells bearing the appropriate T-cell receptor (TCR), whereas complexes of peptide and MHC class II molecules are recognized by CD4-positive-helper-T cells bearing the appropriate TCR.

It is well known that the TCR, the peptide and the MHC are thereby present in a stoichiometric amount of 1:1:1.

For an MHC class I peptide to trigger (elicit) a cellular immune response, it also must bind to an MHC-molecule. This process is dependent on the allele of the MHC-molecule and specific polymorphisms of the amino acid sequence of the peptide. MHC-class-l- binding peptides are usually 8-12 amino acid residues in length and usually contain two conserved residues (“anchors”) in their sequence that interact with the corresponding binding groove of the MHC-molecule. In this way each MHC allele has a “binding motif” determining which peptides can bind specifically to the binding groove. In the MHC class I dependent immune reaction, peptides not only have to be able to bind to certain MHC class I molecules expressed by virally infected cells, they subsequently also have to be recognized by T cells bearing specific T cell receptors (TCR).

Basically, any peptide able to bind an MHC molecule may function as a T-cell epitope. A prerequisite for the induction of an in vitro or in vivo T-cell-response is the presence of a T cell having a corresponding TCR and the absence of immunological tolerance for this particular epitope.

Stimulation of an immune response is dependent upon the presence of antigens recognized as foreign by the host immune system. The discovery of the existence of virus associated antigens has raised the possibility of using a host's immune system to intervene with viral infection. Various mechanisms of harnessing both the humoral and cellular arms of the immune system are currently being explored for immunotherapy. Specific elements of the cellular immune response are capable of specifically recognizing and destroying infected cells. CD8-positive T-cells in particular, which recognize class I molecules of the major histocompatibility complex (MHC)-bearing peptides of usually 8 to 10 amino acid residues derived from proteins or defect ribosomal products (DRIPS) located in the cytosol, play an important role in the response. The MHC-molecules of the human are also designated as human leukocyte-antigens (HLA).

The term “T-cell response” shall relate to the specific proliferation and activation of effector functions induced by a peptide in vitro or in vivo. For MHC class I restricted cytotoxic T cells, effector functions may be lysis of peptide-pulsed, peptide-precursor pulsed or naturally peptide-presenting target cells, secretion of cytokines, preferably Interferon-gamma, TNF-alpha, or IL-2 induced by peptide, secretion of effector molecules, preferably granzymes or perforins induced by peptide, or degranulation.

The term “peptide” is used herein to designate a series of amino acid residues, connected one to the other typically by peptide bonds between the alpha-amino and carbonyl groups of the adjacent amino acids. The peptides are preferably 9 amino acids in length, but can be as short as 8 amino acids in length, and as long as 10, 11, 12, or 13 amino acids or longer, and in case of MHC class II peptides (longer variants of the peptides of the invention) they can be as long as 14, 15, 16, 17, 18, 19 or 20 or more amino acids in length. The peptides can be extended by one amino acid on their N-and/or C-terminus, these extensions of course should not substantially interfere with the activity of those peptides.

Furthermore, the term “peptide” shall include salts of a series of amino acid residues, connected one to the other typically by peptide bonds between the alpha-amino and carbonyl groups of the adjacent amino acids. Preferably, the salts are pharmaceutical acceptable salts of the peptides, such as, for example, the chloride or acetate (trifluoroacetate) salts. It has to be noted that the salts of the peptides according to the present invention differ substantially from the peptides in their state(s) in vivo, as the peptides are not salts in vivo.

Consequently, as used herein, “a pharmaceutically acceptable salt” refers to a derivative of the disclosed peptides wherein the peptide is modified by making acid or base salts of the agent. For example, acid salts are prepared from the free base (typically wherein the neutral form of the drug has a neutral —NH2 group) involving reaction with a suitable acid. Suitable acids for preparing acid salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a peptide are prepared using a pharmaceutically acceptable base such as sodium hydroxide, potassium hydroxide, ammonium hydroxide, calcium hydroxide, trimethylamine or the like. In an especially preferred embodiment, the peptides are—and the pharmaceutical compositions comprise the peptides as—salts of acetic acid (acetates), trifluoro acetates or hydrochloric acid (chlorides).

Another embodiment of the present invention relates to a non-naturally occurring peptide wherein said peptide consists or consists essentially of an amino acid sequence according to SEQ ID NO: 1 to SEQ ID NO: 101 and has been synthetically produced (e.g. synthesized) as a pharmaceutically acceptable salt. Methods to synthetically produce peptides are well known in the art. The salts of the peptides according to the present invention differ substantially from the peptides in their state(s) in vivo, as the peptides as generated in vivo are no salts. The non-natural salt form of the peptide mediates the solubility of the peptide, in particular in the context of pharmaceutical compositions comprising the peptides, e.g. the peptide vaccines as disclosed herein. A sufficient and at least substantial solubility of the peptide(s) is required in order to efficiently provide the peptides to the subject to be treated. Preferably, the salts are pharmaceutically acceptable salts of the peptides. These salts according to the invention include alkaline and earth alkaline salts such as salts of the Hofmeister series comprising as anions PO₄³⁻, SO₄²⁻, CH₃COO⁻, Cl—, Br—, NO₃⁻, ClO₄⁻, I⁻, SCN⁻, and as cations NH₄⁺, Rb⁺, K⁺, Na⁺, Cs⁺, Li⁺, Zn²⁺, Mg²⁺, Ca²⁺, Mn²⁺, Cu²⁺, and Ba²⁺. Particularly salts are selected from (NH₄)₃PO₄, (NH₄)₂HPO₄, (NH₄)H₂PO₄, (NH₄)₂SO₄, NH₄CH₃COO, NH₄Cl, NH₄Br, NH₄NO₃, NH₄ClO₄, NH₄I, NH₄SCN, Rb₃PO₄, Rb₂HPO₄, RbH₂PO₄, Rb₂SO₄, Rb₄CH₃COO, Rb₄Cl, Rb₄Br, Rb₄NO₃, Rb₄ClO₄, Rb₄I, Rb₄SCN, K₃PO₄, K₂HPO₄, KH₂PO₄, K₂SO₄, KCH₃COO, KCl, KBr, KNO₃, KClO₄, KI, KSCN, Na₃PO₄, Na₂HPO₄, NaH₂PO₄, Na₂SO₄, NaCH₃COO, NaCl, NaBr, NaNO₃, NaClO₄, Nal, NaSCN, ZnCI₂ Cs₃PO₄, Cs₂HPO₄, CsH₂PO₄, Cs₂SO₄, CsCH₃COO, CsCI, CsBr, CsNO₃, CsClO₄, CSI, CsSCN, Li₃PO₄, Li₂HPO₄, LiH₂PO₄, Li₂SO₄, LiCH₃COO, LiCl, LiBr, LiNO₃, LiClO₄, LiI, LiSCN, Cu₂SO₄, Mg₃(PO₄)₂, Mg₂HPO₄, Mg(H₂PO₄)₂, Mg₂SO₄, Mg(CH₃COO)₂, MgCl₂, MgBr₂, Mg(NO₃)₂, Mg(ClO₄)₂, MgI₂, Mg(SCN)₂, MnCl₂, Ca₃(PO₄)₂, Ca₂HPO₄, Ca(H₂PO₄)₂, CaSO₄, Ca(CH₃COO)₂, CaCl₂, CaBr₂, Ca(NO₃)₂, Ca(ClO₄)₂, CaI₂, Ca(SCN)₂, Ba₃(PO₄)₂, Ba₂HPO₄, Ba(H₂PO₄)₂, BaSO₄, Ba(CH₃COO)₂, BaCI₂, BaBr₂, Ba(NO₃)₂, Ba(CIO₄)₂, BaI₂, and Ba(SCN)₂. Particularly preferred are NH acetate, MgCl₂, KH₂PO, Na₂SO, KCl, NaCl, and CaCl₂, such as, for example, the chloride or acetate (trifluoroacetate) salts.

A peptide, oligopeptide, protein or polynucleotide coding for such a molecule is “immunogenic” (and thus is an “immunogen” within the present invention), if it is capable of inducing an immune response. In the case of the present invention, immunogenicity is more specifically defined as the ability to induce a T-cell response. Thus, an “immunogen” would be a molecule that is capable of inducing an immune response, and in the case of the present invention, a molecule capable of inducing a T-cell response. In another aspect, the immunogen can be the peptide, the complex of the peptide with MHC, oligopeptide, and/or protein that is used to raise specific antibodies or TCRs against it.

A class I T cell “epitope” requires a short peptide that is bound to a class I MHC receptor, forming a ternary complex (MHC class I alpha chain, beta-2-microglobulin, and peptide) that can be recognized by a T cell bearing a matching T-cell receptor binding to the MHC/peptide complex with appropriate affinity. Peptides binding to MHC class I molecules are typically 8-14 amino acids in length, and most typically 9 amino acids in length.

In humans there are three different genetic loci that encode MHC class I molecules (the MHC-molecules of the human are also designated human leukocyte antigens (HLA)): HLA-A, HLA-B, and HLA-C. HLA-A*01, HLA-A*02, and HLA-B*07 are examples of different MHC class I alleles that can be expressed from these loci.

In a preferred embodiment, the term “nucleotide sequence” refers to a heteropolymer of deoxyribonucleotides. The nucleotide sequence coding for a particular peptide, oligopeptide, or polypeptide may be naturally occurring or they may be synthetically constructed. Generally, DNA segments encoding the peptides, polypeptides, and proteins of this invention are assembled from cDNA fragments and short oligonucleotide linkers, or from a series of oligonucleotides, to provide a synthetic gene that is capable of being expressed in a recombinant transcriptional unit comprising regulatory elements derived from a microbial or viral operon. As used herein the term “a nucleotide coding for (or encoding) a peptide” refers to a nucleotide sequence coding for the peptide including artificial (man-made) start and stop codons compatible for the biological system the sequence is to be expressed by, for example, a dendritic cell or another cell system useful for the production of TCRs. As used herein, reference to a nucleic acid sequence includes both single stranded and double stranded nucleic acid. Thus, for example for DNA, the specific sequence, unless the context indicates otherwise, refers to the single strand DNA of such sequence, the duplex of such sequence with its complement (double stranded DNA) and the complement of such sequence.

The term “coding region” refers to that portion of a gene which either naturally or normally codes for the expression product of that gene in its natural genomic environment, i.e., the region coding in vivo for the native expression product of the gene. The coding region can be derived from a non-mutated (“normal”), mutated or altered gene, or can even be derived from a DNA sequence, or gene, wholly synthesized in the laboratory using methods well known to those of skill in the art of DNA synthesis.

The term “expression product” means the polypeptide or protein that is the natural translation product of the gene and any nucleic acid sequence coding equivalents resulting from genetic code degeneracy and thus coding for the same amino acid(s).

The term “promoter” means a region of DNA involved in binding of RNA polymerase to initiate transcription.

In accordance with the present invention, the term “percent identity” or “percent identical”, when referring to a sequence, means that a sequence is compared to a sequence as claimed or described after alignment of the sequence to be compared (the “Compared Sequence”) with the described or claimed sequence (the “Reference Sequence”). The percent identity is then determined according to the following formula: percent identity=100[1−(C/R)] wherein C is the number of differences between the Reference Sequence and the Compared Sequence over the length of alignment between the Reference Sequence and the Compared Sequence, wherein

(i) each base or amino acid in the Reference Sequence that does not have a corresponding aligned base or amino acid in the Compared Sequence and

(ii) each gap in the Reference Sequence and

(iii) each aligned base or amino acid in the Reference Sequence that is different from an aligned base or amino acid in the Compared Sequence, constitutes a difference and (iv) the alignment has to start at position 1 of the aligned sequences;

and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the herein above calculated percent identity is less than the specified percent identity. A sequence identity can be determined by creating an alignment using, for example, the ClustalW algorithm. Commonly available sequence analysis software, more specifically, Vector NTI, GENETYX or other tools are provided by public databases.

A person skilled in the art will be able to assess, whether T cells induced by a variant of a specific peptide will be able to cross-react with the peptide itself (Appay et al., 2006; Colombetti et al., 2006; Fong et al., 2001; Zaremba et al., 1997).

By a “variant” of the given amino acid sequence the inventors mean that the side chains of, for example, one or two of the amino acid residues are altered (for example by replacing them with the side chain of another naturally occurring amino acid residue or some other side chain) such that the peptide is still able to bind to an HLA molecule in substantially the same way as a peptide consisting of the given amino acid sequence in consisting of SEQ ID NO: 1 to SEQ ID NO: 101. For example, a peptide may be modified so that it at least maintains, if not improves, the ability to interact with and bind to the binding groove of a suitable MHC molecule, such as HLA-A*02 or -DR, and in that way it at least maintains, if not improves, the ability to bind to the TCR of activated T cells. These T cells can subsequently cross-react with cells and kill cells that express a polypeptide that contains the natural amino acid sequence of the cognate peptide as defined in the aspects of the invention. As can be derived from the scientific literature and databases (Rammensee et al., 1999; Godkin et al., 1997), certain positions of HLA binding peptides are typically anchor residues forming a core sequence fitting to the binding motif of the HLA receptor, which is defined by polar, electrophysical, hydrophobic and spatial properties of the polypeptide chains constituting the binding groove. Thus, one skilled in the art would be able to modify the amino acid sequences set forth in SEQ ID NO: 1 to SEQ ID NO: 101, by maintaining the known anchor residues, and would be able to determine whether such variants maintain the ability to bind MHC class I or II molecules. The variants of the present invention retain the ability to bind to the TCR of activated T cells, which can subsequently cross-react with and kill cells that express a polypeptide containing the natural amino acid sequence of the cognate peptide as defined in the aspects of the invention. The original (unmodified) peptides as disclosed herein can be modified by the substitution of one or more residues at different, possibly selective, sites within the peptide chain, if not otherwise stated. Preferably those substitutions are located at the end of the amino acid chain. Such substitutions may be of a conservative nature, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.” Variants are further length variants, where amino acid(s) are added to the “core sequence” of the amino acid sequences set forth in SEQ ID NO: 1 to SEQ ID NO: 101.

Conservative substitutions are herein defined as exchanges within one of the following five groups: Group 1 -small aliphatic, nonpolar or slightly polar residues (Ala, Ser, Thr, Pro, Gly); Group 2-polar, negatively charged residues and their amides (Asp, Asn, Glu, Gin); Group 3-polar, positively charged residues (His, Arg, Lys); Group 4-large, aliphatic, nonpolar residues (Met, Leu, lie, Val, Cys); and Group 5-large, aromatic residues (Phe, Tyr, Trp). If substitutions at more than one position are found to result in a peptide with substantially equivalent or greater antigenic activity as defined herein, then combinations of those substitutions will be tested to determine if the combined substitutions result in additive or synergistic effects on the antigenicity of the peptide. At most, no more than four positions within the peptide would be simultaneously substituted. The amino acid residues that do not substantially contribute to interactions with the T-cell receptor can be modified by replacement with other amino acid whose incorporation does not substantially affect T-cell reactivity and does not eliminate binding to the relevant MHC.

It is possible that MHC class I epitopes, although usually between 8 and 12 amino acids long, are generated by peptide processing from longer peptides or proteins that include the actual epitope. It is preferred that the residues that flank the actual epitope are residues that do not substantially affect proteolytic cleavage necessary to expose the actual epitope during processing. The peptides of the invention can be elongated by up to four amino acids, that is 1 , 2, 3 or 4 amino acids can be added to either end in any combination between 4:0 and 0:4. The amino acids for the elongation/extension can be the peptides of the original sequence of the protein or any other amino acid(s). The elongation can be used to enhance the stability or solubility of the peptides. Thus, the epitopes of the present invention may be identical to naturally occurring tumor-associated or tumor-specific epitopes or may include epitopes that differ by no more than four residues from the reference peptide, as long as they have substantially identical antigenic activity. Of course, the peptide or variant according to the present invention will have the ability to bind to a molecule of the human major histocompatibility complex (MHC) class I or II. Binding of a peptide or a variant to a MHC complex may be tested by methods known in the art.

A particularly preferred embodiment of the invention relates to the peptide or variant according to the present invention, wherein said peptide consists or consists essentially of an amino acid sequence according to any of SEQ ID NO: 1 to SEQ ID NO: 100 or optionally comprises an extension of one N- and/or C-terminal amino acid.

A particularly preferred embodiment of the invention relates to the peptide or variant according to the present invention, wherein said peptide comprises an immune dominant epitope, wherein the amino acid sequence is selected from SEQ ID NO: 1 to 4, 24 to 29, 40, 41, 51 to 55, 67, 68, 80, 87 to 89, and 99 to 101. In another particularly preferred embodiment of the invention the peptide then consists or consists essentially of an amino acid sequence according to SEQ ID NO: 1 to SEQ ID NO: 101. “Consisting essentially of” shall mean that a peptide according to the present invention, in addition to the sequence according to any of SEQ ID NO: 1 to SEQ ID NO 101 or a variant thereof contains additional N- and/or C-terminally located stretches of amino acids that are not necessarily forming part of the peptide that functions as an epitope for MHC molecules.

Nevertheless, these stretches can be important to provide an efficient introduction of the peptide according to the present invention into the cells. In one embodiment of the present invention, the peptide is part of a fusion protein which comprises, for example, the 80 N-terminal amino acids of the HLA-DR antigen-associated invariant chain (p33, in the following “li”) as derived from the NCBI, GenBank Accession number X00497. In other fusions, the peptides of the present invention can be fused to an antibody as described herein, or a functional part thereof, in particular into a sequence of an antibody, so as to be specifically targeted by said antibody, or, for example, to or into an antibody that is specific for dendritic cells as described herein.

In addition, the peptide or variant may be modified further to improve stability and/or binding to MHC molecules in order to elicit a stronger immune response. Methods for such an optimization of a peptide sequence are well known in the art and include, for example, the introduction of reverse peptide bonds or non-peptide bonds. In a reverse peptide bond, the amino acid residues are not joined by peptide (—CO—NH—) linkages but the peptide bond is reversed. Such retro-inverso peptidomimetics may be made using methods known in the art, for example such as those described in Meziere et al (1997) (Meziere et al., 1997). This approach involves making pseudopeptides containing changes involving the backbone, and not the orientation of side chains. Meziere et al. (Meziere et al., 1997) show that for MHC binding and T helper cell responses, these pseudopeptides are useful. Retro-inverse peptides, which contain NH—CO bonds instead of CO—NH peptide bonds, are much more resistant to proteolysis. A non-peptide bond is, for example, —CH₂—NH, —CH₂S—, —CH₂CH₂—, —CH═CH—, —COCH₂—, —CH(OH)CH₂—, and —CH₂SO—. U.S. Pat. No. 4,897,445 provides a method for the solid phase synthesis of non-peptide bonds (—CH₂—NH) in polypeptide chains which involves polypeptides synthesized by standard procedures and the non-peptide bond synthesized by reacting an amino aldehyde and an amino acid in the presence of NaCNBH₃.

A peptide or variant, wherein the peptide is modified or includes non-peptide bonds is a preferred embodiment of the invention. Generally, peptides and variants (at least those containing peptide linkages between amino acid residues) may be synthesized by the Fmoc-polyamide mode of solid-phase peptide synthesis as disclosed by Lukas et al. (Lukas et al., 1981).

Another aspect of the present invention relates to an antibody, in particular a soluble or membrane-bound antibody that specifically binds to the peptide or variant thereof according to the present invention, preferably the peptide or variant thereof according to the present invention when bound to an MHC I or II molecule.

A further aspect of the invention provides an antibody that specifically binds to a human major histocompatibility complex (MHC) class I or II being complexed with a HLA-restricted antigen, wherein the antibody preferably is a polyclonal antibody, monoclonal antibody, bi-specific antibody and/or a chimeric antibody.

It is a further aspect of the invention to provide a method for producing a recombinant antibody specifically binding to a human major histocompatibility complex (MHC) class I or II being complexed with a HLA-restricted antigen, the method comprising: immunizing a genetically engineered non-human mammal comprising cells expressing said human major histocompatibility complex (MHC) class I or II with a soluble form of a MHC class I or II molecule being complexed with said HLA-restricted antigen; isolating mRNA molecules from antibody producing cells of said non-human mammal; producing a phage display library displaying protein molecules encoded by said mRNA molecules; and isolating at least one phage from said phage display library, said at least one phage displaying said antibody specifically binding to said human major histocompatibility complex (MHC) class I or II being complexed with said HLA-restricted antigen. Other methods for producing such antibodies and single chain class I major histocompatibility complexes, as well as other tools for the production of these antibodies are disclosed, for example, in WO 03/068201, WO 2004/084798, WO 01/72768, WO 03/070752, and in publications (Cohen et al., 2003a; Cohen et al., 2003b; Denkberg et al., 2003), which for the purposes of the present invention are all explicitly incorporated by reference in their entireties. Preferably, the antibody is binding with a binding affinity of below 20 nanomolar, preferably of below 10 nanomolar, to the complex, which is also regarded as “specific” in the context of the present invention.

The peptides of the present invention have been shown to be capable of stimulating T cell responses and/or are over-presented and thus can be used for the production of antibodies and/or TCRs, such as soluble TCRs, according to the present invention. Furthermore, the peptides when complexed with the respective MHC can be used for the production of antibodies and/or TCRs, in particular sTCRs, according to the present invention, as well. Respective methods are well known to the person of skill, and can be found in the respective literature as well.

It is a further aspect of the invention to provide a T cell receptor, preferably a recombinant, soluble or membrane-bound T cell receptor, that is reactive with an HLA ligand, such as, for example, a peptide according to the present invention, wherein said ligand is at least 75% identical, preferably at least 88% identical, and most preferred 100% identical to an amino acid sequence according to the present invention. Preferably, the T cell receptor according to the present invention is provided as a soluble molecule, and optionally comprises an effector function, such as an immune stimulating domain or toxin.

The term “T-cell receptor” (abbreviated TCR) refers to a heterodimeric molecule comprising an alpha polypeptide chain (alpha chain) and a beta polypeptide chain (beta chain), wherein the heterodimeric receptor is capable of binding to a peptide antigen presented by an HLA molecule. The term also includes so-called gamma/delta TCRs.

The alpha and beta chains of alpha/beta TCR's, and the gamma and delta chains of gamma/delta TCRs, are generally regarded as each having two “domains”, namely variable and constant domains. The variable domain consists of a concatenation of variable region (V), and joining region (J). The variable domain may also include a leader region (L). Beta and delta chains may also include a diversity region (D). The alpha and beta constant domains may also include C-terminal transmembrane (TM) domains that anchor the alpha and beta chains to the cell membrane. With respect to gamma/delta TCRs, the term “TCR gamma variable domain” as used herein refers to the concatenation of the TCR gamma V (TRGV) region without leader region (L), and the TCR gamma J (TRGJ) region, and the term TCR gamma constant domain refers to the extracellular TRGC region, or to a C-terminal truncated TRGC sequence. Likewise, the term “TCR delta variable domain” refers to the concatenation of the TCR delta V (TRDV) region without leader region (L) and the TCR delta D/J (TRDD/TRDJ) region, and the term “TCR delta constant domain” refers to the extracellular TRDC region, or to a C-terminal truncated TRDC sequence.

As used herein in connect with TCRs of the present description, “specific binding” and grammatical variants thereof are used to mean a TCR having a binding affinity (KD) for a peptide-HLA molecule complex of 100 μM or less.

In an embodiment, a TCR of the present description having at least one mutation in the alpha chain and/or having at least one mutation in the beta chain has modified glycosylation compared to the unmutated TCR.

Alpha/beta heterodimeric TCRs of the present description may have an introduced disulfide bond between their constant domains. Preferred TCRs of this type include those which have a TRAC constant domain sequence and a TRBC1 or TRBC2 constant domain sequence except that Thr 48 of TRAC and Ser 57 of TRBC1 or TRBC2 are replaced by cysteine residues, the said cysteines forming a disulfide bond between the TRAC constant domain sequence and the TRBC1 or TRBC2 constant domain sequence of the TCR.

TCRs of the present description may comprise a detectable label selected from the group consisting of a radionuclide, a fluorophore and biotin. TCRs of the present description may be conjugated to a therapeutically active agent, such as a radionuclide, a chemotherapeutic agent, or a toxin.

In addition, the peptides and/or the TCRs or antibodies or other binding molecules of the present invention can be used to verify a pathologist's diagnosis of a viral infection based on a biopsied or other suitable sample.

The antibodies or TCRs may also be used for in vitro or in vivo diagnostic assays. Generally, the antibody is labeled with a radionucleotide (such as ¹¹¹ln, ⁹⁹Tc, ¹⁴C, ¹³¹l ³H, ³²P or ³⁵S) so that the tumor can be localized using immunoscintiography. In one embodiment, antibodies or fragments thereof bind to the extracellular domains of two or more targets of a protein selected from the group consisting of the above-mentioned proteins, and the affinity value (Kd) is less than 1×10 μM. Antibodies for diagnostic use may be labeled with probes suitable for detection by various imaging methods. Methods for detection of probes include, but are not limited to, fluorescence, light, confocal and electron microscopy; magnetic resonance imaging and spectroscopy; fluoroscopy, computed tomography and positron emission tomography. Suitable probes include, but are not limited to, fluorescein, rhodamine, eosin and other fluorophores, radioisotopes, gold, gadolinium and other lanthanides, paramagnetic iron, fluorine-18 and other positron-emitting radionuclides. Additionally, probes may be bi- or multi-functional and be detectable by more than one of the methods listed. These antibodies may be directly or indirectly labeled with said probes. Attachment of probes to the antibodies includes covalent attachment of the probe, incorporation of the probe into the antibody, and the covalent attachment of a chelating compound for binding of probe, amongst others well recognized in the art. For immunohistochemistry, the disease tissue sample may be fresh or frozen or may be embedded in paraffin and fixed with a preservative such as formalin. The fixed or embedded section contains the sample are contacted with a labeled primary antibody and secondary antibody, wherein the antibody is used to detect the expression of the proteins in situ.

The present description further relates to a method of identifying and isolating a TCR according to the present description, said method comprising incubating PBMCs from HLA-A*02-negative healthy donors with A2/peptide monomers, incubating the PBMCs with tetramer-phycoerythrin (PE) and isolating the high avidity T-cells by fluorescence activated cell sorting (FACS)-Calibur analysis. The present description further relates to a method of identifying and isolating a TCR according to the present description, said method comprising obtaining a transgenic mouse with the entire human TCRab gene loci (1.1 and 0.7 Mb), whose T-cells express a diverse human TCR repertoire that compensates for mouse TCR deficiency, immunizing the mouse with peptide, incubating PBMCs obtained from the transgenic mice with tetramer-phycoerythrin (PE), and isolating the high avidity T-cells by fluorescence activated cell sorting (FACS)-Calibur analysis.

In one embodiment, the present invention provides a method of producing a TCR as described herein, the method comprising culturing a host cell capable of expressing the TCR under conditions suitable to promote expression of the TCR. In one additional aspect, to obtain T-cells expressing TCRs of the present description, nucleic acids encoding TCR-alpha and/or TCR-beta chains of the present description are cloned into expression vectors, such as gamma retrovirus or lentivirus. The recombinant viruses are generated and then tested for functionality, such as antigen specificity and functional avidity. An aliquot of the final product is then used to transduce the target T-cell population (generally purified from patient-PBMCs), which is expanded before infusion into the patient.

In another aspect, to obtain T-cells expressing TCRs of the present invention, TCR RNAs are synthesized by techniques known in the art, e.g., in vitro transcription systems. The in vitro-synthesized TCR RNAs are then introduced into primary CD8+ T-cells obtained from healthy donors by electroporation to re-express tumor specific TCR-alpha and/or TCR-beta chains. The alpha and beta chains of a TCR of the present invention may be encoded by nucleic acids located in separate vectors, or may be encoded by polynucleotides located in the same vector. Achieving high-level TCR surface expression requires that both the TCR-alpha and TCR-beta chains of the introduced TCR be transcribed at high levels. To do so, the TCR-alpha and TCR-beta chains of the present description may be cloned into bi-cistronic constructs in a single vector, which has been shown to be capable of overcoming this obstacle. The use of a viral intraribosomal entry site (IRES) between the TCR-alpha and TCR-beta chains results in the coordinated expression of both chains, because the TCR-alpha and TCR-beta chains are generated from a single transcript that is broken into two proteins during translation, ensuring that an equal molar ratio of TCR-alpha and TCR-beta chains are produced. (Schmitt et al. 2009).

The present invention further relates to a nucleic acid, encoding a peptide or variant thereof according to the present invention, the antibody according to the present invention or the T cell receptor according to the present invention, wherein said nucleic acid is optionally linked to a heterologous promoter sequence. The present invention further relates to the nucleic acid according to the present invention that is DNA, cDNA, PNA, RNA or combinations thereof. The present invention further relates to an expression vector capable of expressing and/or expressing a nucleic acid according to the present invention. In a preferred embodiment, the term “nucleotide sequence” refers to a heteropolymer of deoxyribonucleotides.

The nucleotide sequence coding for a particular peptide, oligopeptide, or polypeptide may be naturally occurring or they may be synthetically constructed. Generally, DNA segments encoding the peptides and other molecules of the invention, such as TCRs and antibodies, polypeptides, and proteins of this invention are assembled from cDNA fragments and short oligonucleotide linkers, or from a series of oligonucleotides, to provide a synthetic gene that is capable of being expressed in a recombinant transcriptional unit comprising regulatory elements derived from a microbial or viral operon.

As used herein, reference to a nucleic acid sequence includes both single stranded and double stranded nucleic acid. Thus, for example for DNA, the specific sequence, unless the context indicates otherwise, refers to the single strand DNA of such sequence, the duplex of such sequence with its complement (double stranded DNA) and the complement of such sequence. The term “coding region” refers to that portion of a gene which either naturally or normally codes for the expression product of that gene in its natural genomic environment, i.e., the region coding in vivo for the native expression product of the gene.

As used herein the term “a nucleotide coding for (or encoding) a peptide” refers to a nucleotide sequence coding for the molecule of the invention including artificial (man-made) start and stop codons compatible for the biological system the sequence is to be expressed by, for example, a dendritic cell or another cell system useful for the production of TCRs. The coding region can be derived from a non-mutated (“normal”), mutated or altered gene, or can even be derived from a DNA sequence, or gene, wholly synthesized in the laboratory using methods well known to those of skill in the art of DNA synthesis. The term “expression product” means the polypeptide or protein that is the natural translation product of the gene and any nucleic acid sequence coding equivalents resulting from genetic code degeneracy and thus coding for the same amino acid(s). The term “fragment”, when referring to a coding sequence, means a portion of DNA comprising less than the complete coding region, whose expression product retains essentially the same biological function or activity as the expression product of the complete coding region. The term “promoter” means a region of DNA involved in binding of RNA polymerase to initiate transcription.

A variety of methods have been developed to link polynucleotides, especially DNA, to vectors for example via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules. Synthetic linkers containing one or more restriction sites provide an alternative method of joining the DNA segment to vectors. Synthetic linkers containing a variety of restriction endonuclease sites are commercially available from a number of sources including International Biotechnologies Inc. New Haven, Conn., USA. A desirable method of modifying the DNA encoding the polypeptide of the invention employs the polymerase chain reaction as disclosed by Saiki R K, et al. (Saiki et al., 1988). This method may be used for introducing the DNA into a suitable vector, for example by engineering in suitable restriction sites, or it may be used to modify the DNA in other useful ways as is known in the art. If viral vectors are used, pox- or adenovirus vectors are preferred.

Nucleic acids encoding molecules, such as TCRs or antibodies, of the present description may be codon optimized to increase expression from a host cell. Redundancy in the genetic code allows some amino acids to be encoded by more than one codon, but certain codons are less “optimal” than others because of the relative availability of matching tRNAs as well as other factors (Gustafsson et al., 2004). Modifying the, for example, TCR-alpha and TCR-beta gene sequences such that each amino acid is encoded by the optimal codon for mammalian gene expression, as well as eliminating mRNA instability motifs or cryptic splice sites, has been shown to significantly enhance TCR-alpha and TCR-beta gene expression (Scholten et al., 2006).

Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the molecule of the invention, which can then be recovered. Many expression systems are known, including bacteria (for example E. coli and Bacillus subtilis), yeasts (for example Saccharomyces cerevisiae), filamentous fungi (for example Aspergillus spec), plant cells, animal cells and insect cells. Preferably, the system can be mammalian cells such as CHO cells available from the ATCC Cell Biology Collection. A typical mammalian cell vector plasmid for constitutive expression comprises the CMV or SV40 promoter with a suitable poly A tail and a resistance marker, such as neomycin.

In another embodiment two or more peptides or peptide variants of the invention are encoded and thus expressed in a successive order (similar to “beads on a string” constructs). In doing so, the peptides or peptide variants may be linked or fused together by stretches of linker amino acids, or may be linked without any additional peptide(s) between them. These constructs can also be used for antiviral therapy, and may induce immune responses both involving MHC I and MHC II.

The present invention also relates to a host cell transformed or transfected with a polynucleotide vector construct of the present invention. The host cell can be either prokaryotic or eukaryotic. Bacterial cells may be preferred prokaryotic host cells in some circumstances and typically are a strain of E. coli such as, for example, the £. coli strains DH5. Preferred eukaryotic host cells include yeast, insect and mammalian cells, preferably vertebrate cells such as those from a mouse, rat, monkey or human fibroblastic and colon cell lines. Yeast host cells include YPH499, YPH500 and YPH501. Preferred mammalian host cells include Chinese hamster ovary (CHO) cells, NIH Swiss mouse embryo cells NIH/3T3, monkey kidney-derived COS-1 cells, and 293 cells which are human embryonic kidney cells. Preferred insect cells are Sf9 cells which can be transfected with baculovirus expression vectors. An overview regarding the choice of suitable host cells for expression can be found in literature known to the person of skill. Transformation of appropriate cell hosts with a DNA construct of the present invention is accomplished by well-known methods that typically depend on the type of vector used. With regard to transformation of prokaryotic host cells, see, for example, Cohen et al. (Cohen et al., 1972) and (Green and Sambrook, 2012).

Transformation of yeast cells is described in Sherman et al. (Sherman et al., 1986). With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA. Electroporation is also useful for transforming and/or transfecting cells and is well known in the art for transforming yeast cell, bacterial cells, insect cells and vertebrate cells.

The DNA (or in the case of retroviral vectors, RNA) may then be expressed in a suitable host to produce a molecule of the invention, e.g. comprising the peptide or variant of the invention. Thus, the DNA, preferably encoding the peptide or variant of the invention, may be used in accordance with known techniques, appropriately modified in view of the teachings contained herein, to construct an expression vector, which is then used to transform an appropriate host cell for the expression and production of the polypeptide of the invention. The DNA (or in the case of retroviral vectors, RNA) encoding a polypeptide constituting the molecule of the invention may be joined to a wide variety of other DNA sequences for introduction into an appropriate host. The companion DNA will depend upon the nature of the host, the manner of the introduction of the DNA into the host, and whether episomal maintenance or integration is desired.

Successfully transformed cells, i.e. cells that contain a DNA construct of the present invention, can be identified by well-known techniques such as PCR. Alternatively, the presence of the protein in the supernatant can be detected using antibodies. It will be appreciated that certain host cells of the invention are useful in the preparation of the molecules of the invention, for example bacterial, yeast and insect cells. However, other host cells may be useful in certain therapeutic methods. For example, antigen-presenting cells, such as dendritic cells, may usefully be used to express the peptides of the invention such that they may be loaded into appropriate MHC molecules. Thus, the current invention provides a host cell comprising a nucleic acid or an expression vector according to the invention. In a preferred embodiment, the host cell is an antigen presenting cell, in particular a dendritic cell or antigen presenting cell.

The present invention further relates to a method for producing a peptide according to the present invention, said method comprising culturing the host cell according to the present invention, and isolating the peptide or other molecule of the invention from said host cell or its culture medium. The present invention further relates to said method according to the present invention, wherein the peptide antigen is loaded onto class I or II MHC molecules expressed on the surface of a suitable antigen-presenting cell or artificial antigen-presenting cell by contacting a sufficient amount of the antigen with an antigen-presenting cell. The present invention further relates to the method according to the present invention, wherein said antigen-presenting cell comprises an expression vector capable of expressing or expressing said peptide containing SEQ ID NO: 1 to SEQ ID NO: 101. Another aspect of the present invention includes an in vitro method for producing activated T cells, the method comprising contacting in vitro T cells with antigen loaded human MHC molecules expressed on the surface of a suitable antigen-presenting cell for a period of time sufficient to activate the T cell in an antigen specific manner, wherein the antigen is a peptide according to the invention. Preferably a sufficient amount of the antigen is used with an antigen-presenting cell.

Another aspect of the invention relates to an activated T lymphocyte, produced by the method according to the present invention that selectively recognizes a cell which presents a polypeptide comprising an epitope amino acid sequence as disclosed herein. The activated T cells that are directed against the peptides of the invention are useful in therapy. Thus, a further aspect of the invention provides activated T cells obtainable by the foregoing methods of the invention. Activated T cells, which are produced by the above method, will selectively recognize a cell that aberrantly expresses a polypeptide that comprises an amino acid sequence of SEQ ID NO: 1 to SEQ ID NO: 101. Preferably, the T cell recognizes the cell by interacting through its TCR with the HLA/peptide-complex (for example, binding). The T cells of the present invention may be used as active ingredients of a therapeutic composition.

Another aspect of the present invention includes the use of the peptides complexed with MHC to generate a T-cell receptor whose nucleic acid is cloned and is introduced into a host cell, preferably a T cell. This engineered T cell can then be transferred to a patient for therapy of viral infection. Any molecule of the invention, i.e. the peptide, nucleic acid, antibody, expression vector, cell, activated T cell, T-cell receptor or the nucleic acid encoding these, is useful for the treatment of disorders, characterized by cells escaping an immune response. Therefore, any molecule of the present invention may be used as medicament or in the manufacture of a medicament. The molecule may be used by itself or combined with other molecule(s) of the invention or (a) known molecule(s).

Another aspect of the present invention relates to a pharmaceutical composition comprising at least one active ingredient selected from the group consisting of the peptide or variant thereof according to the present invention, the antibody according to the present invention, the T cell receptor according to the present invention, the nucleic acid according to the present invention, the expression vector according to the present invention, the host cell according to the present invention or the activated T lymphocyte according to the present invention, and a pharmaceutically acceptable carrier, and optionally additional pharmaceutically acceptable excipients and/or stabilizers. A “pharmaceutical composition” is a composition suitable for administration to a human being in a medical setting. Preferably, a pharmaceutical composition is sterile and produced according to GMP guidelines. The pharmaceutical composition may be prepared for intravenous (i.v.) injection, sub-cutaneous (s.c.) injection, intradermal (i.d.) injection, intraperitoneal (i.p.) injection, intramuscular (i.m.) injection.

More preferably, the pharmaceutical composition is in the form of a peptide vaccine. Methods for formulating peptide vaccines are known to the person of skill and disclosed in the respective literature. It may be administered directly into the patient, into the affected organ or systemically i.d., i.m., s.c., i.p. and i.v., or applied ex vivo to cells derived from the patient or a human cell line which are subsequently administered to the patient, or used in vitro to select a subpopulation of immune cells derived from the patient, which are then re-administered to the patient. If the nucleic acid is administered to cells in vitro, it may be useful for the cells to be transfected so as to co-express immune-stimulating cytokines, such as interleukin-2. The peptide may be substantially pure, or combined with an immune-stimulating adjuvant (see below) or used in combination with immune-stimulatory cytokines, or be administered with a suitable delivery system, for example liposomes. The peptide may also be conjugated to a suitable carrier such as keyhole limpet haemocyanin (KLH) or mannan (see WO 95/18145). The peptide may also be tagged, may be a fusion protein, or may be a hybrid molecule. The peptides whose sequence is given in the present invention are expected to stimulate CD4 or CD8 T cells. However, stimulation of CD8 T cells is more efficient in the presence of help provided by CD4 T-helper cells. Thus, for MHC Class I epitopes that stimulate CD8 T cells the fusion partner or sections of a hybrid molecule suitably provide epitopes which stimulate CD4-positive T cells. CD4- and CD8-stimulating epitopes are well known in the art and include those identified in the present invention.

In one aspect, the vaccine comprises at least one peptide having the amino acid sequence set forth SEQ ID NO: 1 to SEQ ID NO: 101, and at least one additional peptide, preferably two to 50, more preferably two to 25, even more preferably two to 20 and most preferably two, three, four, five, six, seven, eight, nine, ten, eleven, twelve, thirteen, fourteen, fifteen, sixteen, seventeen or eighteen peptides. The peptide(s) may be derived from one or more specific TAAs and may bind to MHC class I or II molecules.

The vaccine of the invention may also include one or more adjuvants. Adjuvants are substances that non-specifically enhance or potentiate the immune response (e.g., immune responses mediated by CD8-positive T cells and helper-T (TH) cells to an antigen, and would thus be considered useful in the medicament of the present invention. Suitable adjuvants include, but are not limited to, 1018 ISS, aluminum salts, AMPLIVAX®, AS15, BCG, CP-870,893, CpG7909, CyaA, dSLIM, flagellin or TLRS ligands derived from flagellin, FLT3 ligand, GM-CSF, IC30, IC31, Imiquimod (ALDARA®), resiquimod, ImuFact IMP321, Interleukins as IL-2, IL-13, IL-21, Interferon- alpha or -beta, or pegylated derivatives thereof, IS Patch, ISS, ISCOMATRIX, ISCOMs, Juvlmmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactid co-glycolid) [PLG]-based and dextran microparticles, talactoferrin SRL172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants, such as Freund's or GM-CSF, are preferred. Several immunological adjuvants (e.g., MF59) specific for dendritic cells and their preparation have been described previously (Allison and Krummel, 1995). Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) and acting as immunoadjuvants (e.g., IL-12, IL-15, IL-23, IL-7, IFN-alpha. IFN-beta) (Gabrilovich et al., 1996). CpG immunostimulatory oligonucleotides have also been reported to enhance the effects of adjuvants in a vaccine setting.

The pharmaceutical compositions comprise the peptides either in the free form or in the form of a pharmaceutically acceptable salt (see also above). In an aspect, a peptide described herein is in the form of a pharmaceutically acceptable salt. In another aspect, a peptide in the form of a pharmaceutical salt is in crystalline form. A pharmaceutically acceptable salt described herein refers to salts which possess toxicity profiles within a range that is acceptable for pharmaceutical applications.

The present invention further relates to a method for producing a personalized anti-viral vaccine, said method comprising: a) identifying at least one HCMV-associated peptide according to any one of SEQ ID NO: 1 to SEQ ID NO: 101 in a sample from said individual patient; b) selecting at least one peptide as identified in said sample from step a), and c) formulating the at least one peptide as selected in step b) into a personalized anti-viral vaccine.

In said method, for an individual patient at least one peptide selected from a selection of pre-screened epitope peptides is selected for suitability in the individual patient. The method could also be adapted to produce T cell clones for down-stream applications, such as TCR isolations, or soluble antibodies, and other treatment options. In an aspect, the peptides are pre-screened for immunogenicity before being included in the warehouse. By way of example, and not limitation, the immunogenicity of the peptides included in the selection is determined by a method comprising in vitro T-cell priming through repeated stimulations of CD8+ T cells from healthy donors with artificial antigen presenting cells loaded with peptide/MHC complexes and anti-CD28 antibody.

In contrast to multi-peptide cocktails with a fixed composition as currently developed, the selection allows a significantly higher matching of the actual presentation of antigens with the vaccine. Selected single or combinations of several “off-the-shelf peptides will be used for each patient in a multitarget approach. In an aspect, the peptides are selected for inclusion in the vaccine based on their suitability for the individual patient based on the method according to the present invention as described herein, or as below. The HLA phenotype, transcriptomic and peptidomic data is gathered from the patient's tumor material, and blood samples to identify the most suitable peptides for each patient containing selection and patient-unique (i.e. mutated) TUMAPs. Those peptides will be chosen, which are selectively or over-expressed in the patient and, where possible, show strong in vitro immunogenicity if tested with the patients' individual PBMCs. In addition to, or as an alternative to, selecting peptides using a selection (database) model, peptides may be identified in the patient de novo, and then included in the vaccine. Once the peptides for a personalized peptide based vaccine are selected, the vaccine is produced. The vaccine preferably is a liquid formulation consisting of the individual peptides dissolved in between 20-40% DMSO, preferably about 30-35% DMSO, such as about 33% DMSO. Each peptide to be included into a product is dissolved in DMSO. The concentration of the single peptide solutions has to be chosen depending on the number of peptides to be included into the product. For example, the single peptide-DMSO solutions are mixed in equal parts to achieve a solution containing all peptides to be included in the product with a concentration of about 2.5 mg/ml per peptide.

The present invention further relates to the peptide or variant thereof according to the invention, the antibody according to the invention, the T cell receptor according to the invention, the nucleic acid according to the invention, the expression vector according to the invention, the host cell according to the invention or the activated T lymphocyte according to the invention, the pharmaceutical composition according to the invention, or the vaccine as produced according to the invention for use in medicine.

The peptides of the present invention as well as other molecules of the invention (such as cells, antibodies and TCRs) are useful for generating an immune response in a patient by which virally infected cells can be destroyed. An immune response in a patient can be induced by direct administration of the described peptides or suitable precursor substances (e.g. elongated peptides, proteins, or nucleic acids encoding these peptides) to the patient, ideally in combination with an agent enhancing the immunogenicity (i.e. an adjuvant). The immune response originating from such a therapeutic vaccination can be expected to be highly specific against infected cells because the target peptides of the present invention are not presented on normal tissues in comparable copy numbers, preventing the risk of undesired autoimmune reactions against normal cells in the patient.

The present invention further relates to the peptide or variant thereof according to the invention, the antibody according to the invention, the T cell receptor according to the invention, the nucleic acid according to the invention, the expression vector according to the invention, the host cell according to the invention or the activated T lymphocyte according to the invention, the pharmaceutical composition according to the invention, or the vaccine as produced according to the invention for use in the diagnosis (e.g. as above) and/or treatment of HCMV infection, or for use in the manufacture of a medicament against HCMV infection.

In addition to being useful for treating infection, the peptides of the present invention are also useful as diagnostics. Since the peptides were generated from infected cells and since it was determined that these peptides are not or at lower levels present in normal tissues, these peptides can be used to diagnose the presence of a viral infection. The presence of claimed peptides on tissue biopsies in blood samples can assist a pathologist in diagnosis of viral infection. Detection of certain peptides by means of antibodies, mass spectrometry or other methods known in the art can tell the pathologist that the tissue sample is infected, or can be used as a biomarker for HCMV. Presence of groups of peptides can enable classification or sub-classification of diseased tissues.

The present invention further relates to the peptide or variant thereof according to the invention, the antibody according to the invention, the T cell receptor according to the invention, the nucleic acid according to the invention, the expression vector according to the invention, the host cell according to the invention or the activated T lymphocyte according to the invention, the pharmaceutical composition according to the invention, or the vaccine as produced according to the invention for use according to the invention, wherein said HCMV infection exhibits a co-morbidity with cancer, inflammatory diseases, hypertensive diseases, and pulmonary diseases. This aspect involves co-treatment of the infection with other suitable pharmaceuticals that are known to the person of skill.

Another aspect then relates to a method for treating HCMV infection in target cells in a patient, wherein said target cells present at least one peptide comprising an amino acid sequence according to the invention, comprising administering to said patient an effective amount of activated T lymphocytes according to the invention, the pharmaceutical composition according to the invention, and/or of the vaccine as produced according to the invention.

The molecules of the invention, like the antibodies, TCRs, nucleic acids, peptides or cells can be administered to the subject, patient, or cell by injection (e.g., intravenous, intraperitoneal, subcutaneous, intramuscular), or by other methods such as infusion that ensure its delivery to the bloodstream in an effective form. The molecules of the invention, like the antibodies, TCRs, nucleic acids, peptides or cells may also be administered by intratumoral or peritumoral routes, to exert local as well as systemic therapeutic effects. Local or intravenous injection is preferred.

Effective dosages and schedules for administering the molecules may be determined empirically, and making such determinations is within the skill in the art. Those skilled in the art will understand that the dosage of antibodies that must be administered will vary depending on, for example, the subject that will receive the antibody, the route of administration, the particular type of antibody used and other drugs being administered. A typical daily dosage of the antibody used alone might range from about 1 \hat{ } g/kg to up to 100 mg/kg of body weight or more per day, depending on the factors mentioned above. Following administration of an antibody, preferably for treating viral infection, the efficacy of the therapeutic antibody can be assessed in various ways well known to the skilled practitioner. A therapeutically-administered molecule, such as a peptide or antibody, that arrests viral infection, and/or prevents the development of newly infected cells, compared to the disease course that would occur in the absence of molecule (e.g. peptide or antibody) administration, is an efficacious molecule (e.g. peptide or antibody) for treatment of viral infection.

Another aspect then relates to a therapeutic or diagnostic kit comprising: a) a container comprising a peptide or variant thereof according to the invention, the antibody according to the invention, the T cell receptor according to the invention, the nucleic acid according to the invention, the expression vector according to the invention, the host cell according to the invention, or the activated T lymphocyte according to the invention, the pharmaceutical composition according to the invention, or the vaccine as produced according to the invention, in solution or in lyophilized form; b) optionally, a second container containing a diluent or reconstituting solution for the lyophilized formulation; c) optionally, at least one additional peptide selected from the group consisting of SEQ ID NO: 1 to SEQ ID NO: 101, and d) optionally, instructions for (i) use of the solution or (ii) reconstitution and/or use of the lyophilized formulation. The kit may further comprise one or more of (iii) a buffer, (iv) a diluent, (v) a filter, (vi) a needle, or (v) a syringe. The container is preferably a bottle, a vial, a syringe or test tube; and it may be a multi-use container. The pharmaceutical composition is preferably lyophilized.

Kits of the present invention preferably comprise a lyophilized formulation of the present invention in a suitable container and instructions for its reconstitution and/or use. Suitable containers include, for example, bottles, vials (e.g. dual chamber vials), syringes (such as dual chamber syringes) and test tubes. The container may be formed from a variety of materials, such as glass or plastic. Preferably the kit and/or container contain/s instructions on or associated with the container that indicates directions for reconstitution and/or use. For example, the label may indicate that the lyophilized formulation is to be reconstituted to peptide concentrations as described above. The label may further indicate that the formulation is useful or intended for subcutaneous administration. The container holding the formulation may be a multi-use vial, which allows for repeat administrations (e.g., from 2-6 administrations) of the reconstituted formulation. The kit may further comprise a second container comprising a suitable diluent (e.g., sodium bicarbonate solution). Upon mixing of the diluent and the lyophilized formulation, the final peptide concentration in the reconstituted formulation is preferably at least 0.15 mg/mL/peptide (=75 μg) and preferably not more than 3 mg/mL/peptide (=1500 μg). The kit may further include other suitable materials, including other buffers, diluents, filters, needles, syringes, and package inserts with instructions for use.

Kits of the present invention may comprise a single container that contains the formulation of the pharmaceutical composition(s) according to the present invention with or without other components (e.g., other compounds or pharmaceutical compositions of these other compounds) or may comprise distinct containers for each component.

Preferably, kits of the invention include a formulation of the invention packaged for use in combination with the co-administration of a second compound (such as adjuvants (e.g. GM-CSF), a chemotherapeutic agent, a natural product, a hormone or antagonist, an anti-angiogenesis agent or inhibitor, an apoptosis-inducing agent or a chelator) or a pharmaceutical composition thereof. The components of the kit may be pre-complexed or each component may be in a separate distinct container prior to administration to a patient. The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container. The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid. Preferably, a therapeutic kit will contain an apparatus (e.g., one or more needles, syringes, eye droppers, pipette, etc.), which enables administration of the agents of the invention that are components of the present kit. In addition to therapeutic kits, diagnostic kits may contain labelled compounds of the invention, as well as materials suitable for detection in the context of a diagnostic method.

The present formulation can be one that is suitable for administration of the peptides by any acceptable route such as oral (enteral), nasal, ophthal, subcutaneous, intradermal, intramuscular, intravenous or transdermal. Preferably, the administration is s.c, and most preferably i.d. administration may be by infusion pump.

Various immune evasion strategies by HCMV strongly interfere with the HLA-I presentation of viral peptides on the host cell surface. However, about 10% of the memory T-cell compartment of seropositive individuals consists of HCMV-specific T cells (18) and this number can dramatically rise in elderly (38).

With the inventors' approach the inventors were able to bypass immune evasion by the US6 family genes and, for the first time, show the direct isolation and identification of physiologically relevant HCMV-specific HLA ligands by mass spectrometry. The inventors' data clearly confirm that memory T cell responses towards peptides derived from viral proteins of all expression stages and functional areas can be detected in immunocompetent HCMV carriers. The fact that memory T cells specific for a significant proportion of these ligands are present in HCMV-seropositive individuals clearly indicates priming of naïve T cells against these antigens at any time during infection.

By now, the initial perception that HCMV T-cell responses are directed only against few immunodominant antigens has been challenged by several groups. Using predictive bioinformatics and functional T cell assays, Elkington et al. identified numerous novel HCMV peptide epitopes from a heterogeneous group of 14 preselected proteins (pp28, pp50, pp65, pp150, pp71, gH, gB, IE1, US2, US3, US6, US11, UL16, and UL18) as T cell targets (16). Sylwester et al. analyzed overlapping 15-mer peptides from 213 HCMV open reading frames (ORFs) by cytokine flow cytometry. Of the tested ORFs, 70% were shown to be immunogenic for CD4 and/or CD8 T cells. Immunogenicity was influenced only modestly by ORF expression kinetics and function (18). Interestingly, besides pp65 and IE1, UL48 was the only tested ORF recognized by CD8+ T cells of more than 50% of tested donors. In contrast to that particular finding, from a total of 103 identified immunogenic HCMV-derived peptides, 26 epitopes, representing 23 different proteins, were classified as dominant in the inventors' study. The inventors found immunogenic peptides from 65 HCMV proteins, confirming previous findings that HCMV-specific CD8+ T-cell immunity in healthy virus carriers is based on a broad repertoire of HCMV antigens. The inventors were able to validate seven previously known dominant epitopes, but some well-established epitopes such as VLEETSVML_{UL123} (A*02) (SEQ ID NO: 112), YSEHPTFTSQY_{UL83} (A*01) (SEQ ID NO: 109), RPHERNGFTVL_{UL83} (B*07) (SEQ ID NO: 102), and ELRRKMMYM_{UL123} (B*08) (SEQ ID NO: 103) were not detected in the inventors' assays. While the inventors' approach has technical limits also other reasons for the lack of detection of some dominant epitopes are imaginable. The HLA-I antigen presentation pathway could be insufficient for processing of specific peptides in the infected fibroblasts. To assess this, in future studies the inventors will apply IFNγ to induce the HLA-I antigen presentation machinery prior to infection, as IFNγ induction strongly improves antigen presentation to CD8+ T cells (39). In addition, ligandome analysis at different time-points during infection could yield peptides of different quality. The inventors observed strongly induced numbers of HLA-B*44:02 ligands in ΔUS2-6 infected cells, i.e. in the presence of US11 expression. This was indeed surprising and the inventors have begun to address the molecular mechanisms behind this phenomenon. Thus, immunoevasins do not only affect the efficiency, but also the quality of antigen presentation. Therefore, it should be taken into account, that expression of one or combinations of several immunoevasins could result in different HLA-I ligandome qualities. Finally, it cannot be excluded that the ΔUS2-11 deletion mutant virus still express factors that interfere with HLA-I peptide loading and presentation.

The broad CD8+ T-cell response against HCMV detected in healthy donors clearly shows that inhibition of HLA-I antigen presentation by immunoevasins is not sufficient to prevent the induction of CD8+ T cells (17), emphasizing the role of priming of CD8+ T cells through cross-presentation (40). However, HCMV will have a major impact, not only quantitatively, but also qualitatively on antigen processing and presentation in the infected cells. This could partly explain why a large portion of HCMV-derived ligands fail to elicit a memory response in seropositive donors. Also, different expression/presentation patterns in different cell types could have an effect on memory responses (41, 42). Such non-immunogenic ligands might not be processed and presented during an infection in vivo, or are not recognized by naïve T cells. Furthermore, donors might lack specific naïve T cells leaving a hole in the T-cell repertoire (43-45). Despite the large number of non-immunogenic HLA-ligands identified, all these peptides are naturally presented on HLA molecules, providing a solid foundation for epitope screening. By employing in silico analyses only, it would have been necessary to screen thousands of peptides to identify the inventors' set of epitopes, as opposed to 368 in the inventors' approach.

Infection of various cell cultures expressing distinct HLA alleles with different HCMV deletion mutants will allow for deeper and broader insights into the quality of viral CD8+ T-cell targets. Moreover, methods such as ribosome profiling, will enable the identification of novel open reading frames that might be a source of T-cell epitopes (46, 47). The presence of a broad range of specific memory T cells in healthy seropositive individuals suggests that strategies employing subdominant epitopes and targeting multiple antigens in vaccination and cellular therapies may be beneficial for sustainable virus control (48-50). Therefore, the identification of a large number of immunogenic HCMV-derived cytotoxic T-cell targets for the most frequent HLA restrictions is, in the inventors' opinion, indispensable for the development and improvement of such therapies.

In the context of the present invention, the use of HCMV gene deletion mutants lacking various immunoevasins, for the first time enabled the direct isolation and mass spectrometric identification of roughly 380 HCMV-specific HLA-I peptide ligands eluted from twelve different HLA allotypes. Of these peptides 28% induced memory T-cell responses with multifunctional (IFNy, TNFα, CD107a) effector functions in HCMV-positive donors. Finally, real-time cytotoxicity assays demonstrated highly effective cell lysis of HCMV-infected target cells by peptide-specific CD8+ T-cell clones in vitro.

These results confirm that viral HLA-I ligands eluted from infected fibroblast cell cultures reflect physiological peptide processing and presentation mechanisms and are able to induce immunity against HCMV. Therefore, these peptides present novel targets for the treatment of HCMV-associated pathologies by antigen-specific immunotherapy.

In summary, the present invention presents a novel strategy, which enables the direct identification of HCMV-derived T-cell epitopes by mass spectrometry. The inventors provide a panel of novel T-cell epitopes and present evidence for their involvement in physiological immune control of HCMV infections. The inventors' study reveals new targets and provides important insights for the management of CMV-associated pathologies by antigen-specific immunotherapy.

The present invention will now be described with reference to the following non-limiting examples, nevertheless, without being limited thereto. For the purposes of the present invention, all references as cited herein are incorporated by reference in their entireties.

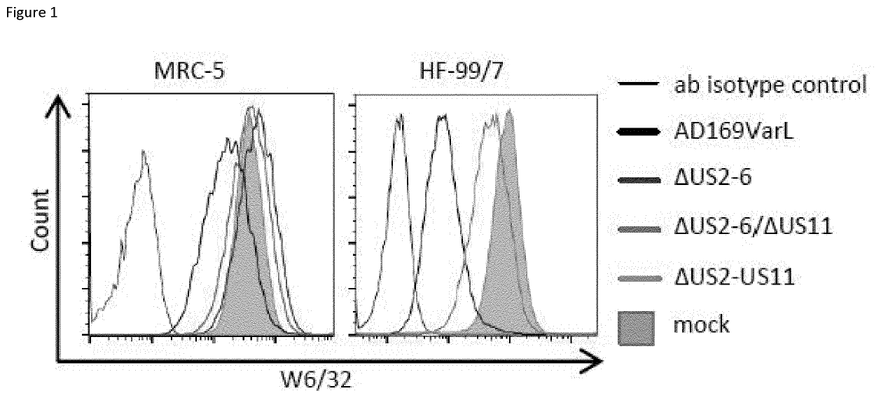

FIG. 1 shows that deletion of the genes US2-US11 allows high level of HLA-I expression. a) MRC-5 or HF-99/7 fibroblasts were mock treated or infected with AD169VarL wild-type virus or deletion mutants with an MOI of 5. Cell surface expression of HLA-I (W6/32) was analyzed by flow cytometry at 48 h.p.i. b) The rate of infection was determined using the cells from (a). The cells were permeabilized and treated with Fc-FITC, which binds to the HCMV encoded Fc-receptors (vFcR). FITC levels were determined by flow cytometry.

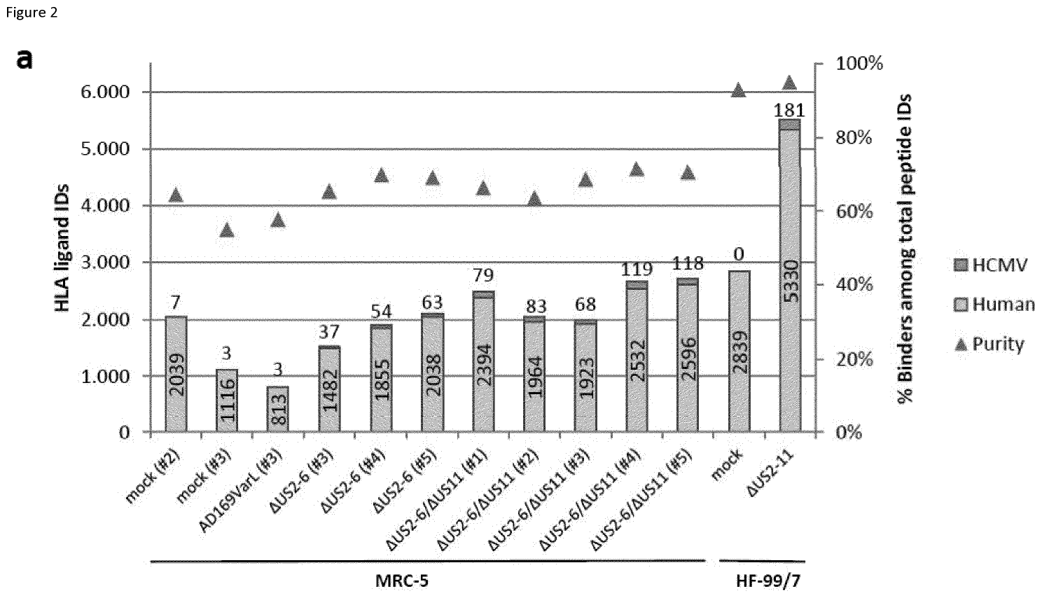

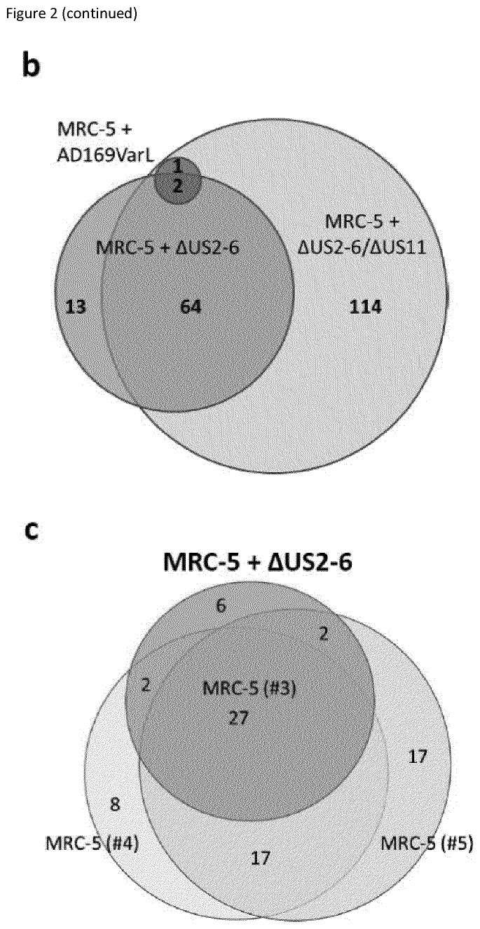

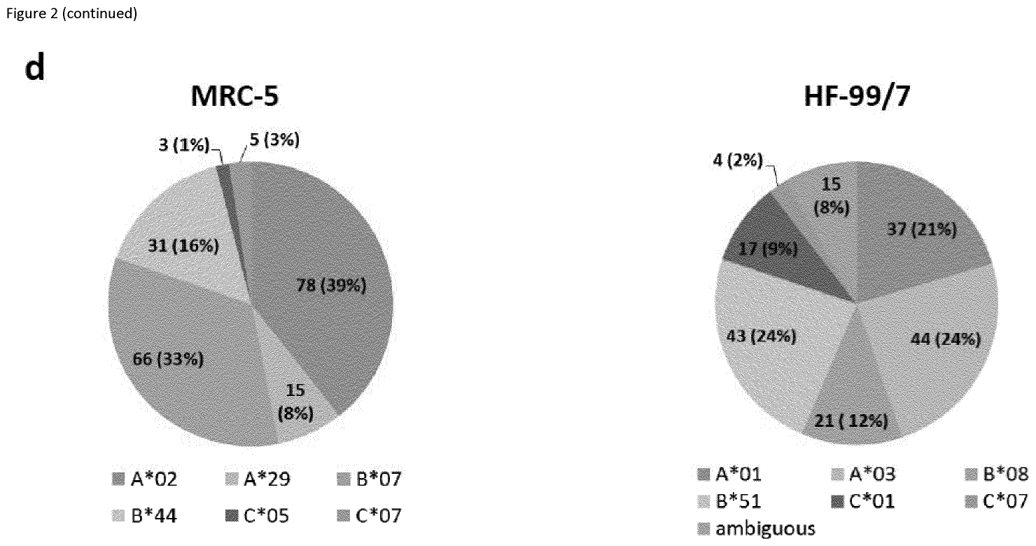

FIG. 2 shows the identification of HCMV-derived HLA ligands from MRC-5 lung fibroblasts by LC-MS/MS. a) Overview of HLA ligand identifications obtained by LC-MS/MS analysis of MRC-5 cells after mock treatment (n=2 independent experiments), infection with AD169VarL (n=1), infection with the deletion viruses AD169 ΔUS2-6 (n=3) or ΔUS2-6/ΔUS11 (n=5). Identified ligands of mock treated (n=1) or AD169 ΔUS2-11 infected (n=1) HF-99/7 cells are depicted on the right side. Peptide identifications were defined as HLA ligands if they showed predicted HLA binding defined as NetMHC IC50≤500 nM and/or normalized SYFPEITHI scores≥50%. The purity of HLA ligand extracts (i.e. the ratio of predicted binders/total peptide identifications) of the individual HLA ligand elutions is indicated by red triangles. b) Overlap analysis of the combined datasets of HCMV individual HLA ligands identified on MRC-5 cells infected with the three different virus variants. c) Overlap of HCMV-derived HLA ligands identified in three independent experiments using MRC-5 cells infected with the deletion virus ΔUS2-6. d) Distribution of HLA restrictions among the 198 (MRC-5) and 181 (HF-99/7) unique HCMV-derived HLA ligands identified in total. Abbreviations: IDs, identifications.

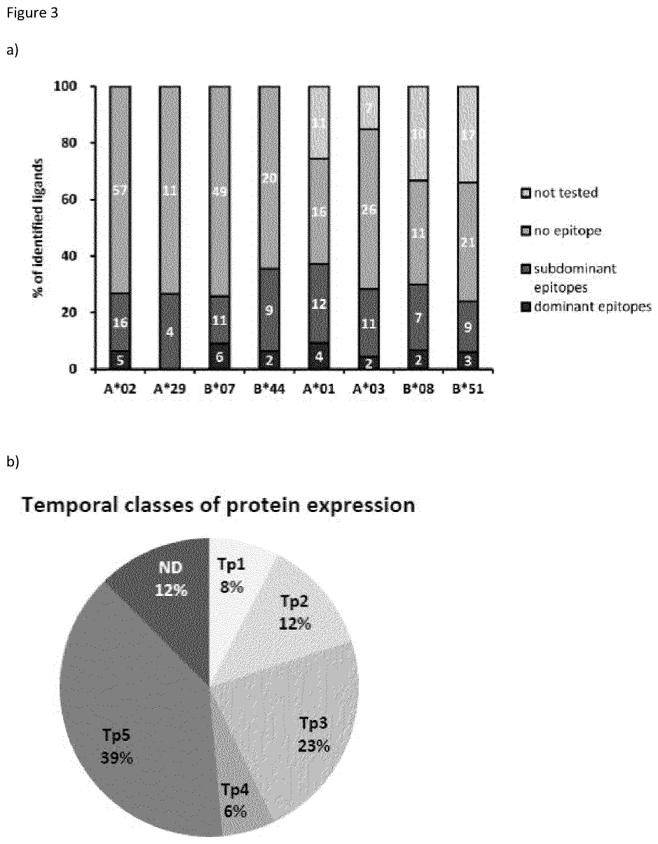

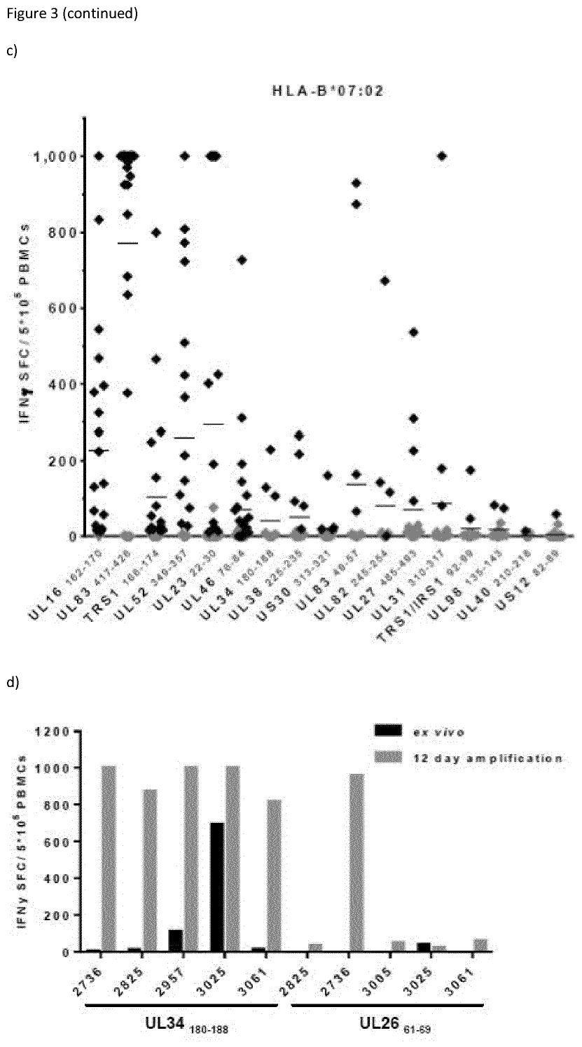

FIG. 3 shows the identification and characterization of naturally presented T-cell epitopes by ELISpot. HCMV ligands were tested for memory T-cell response by IFNγ ELISpot with PBMCs of healthy, seropositive donors. a) Distribution of dominant and subdominant HCMV ligands restricted to HLA-A and -B 19 allotypes. b) Proportion of epitope source proteins assigned to five different temporal classes of protein expression according to Weekes et al. (36). Source proteins not assigned to one of those classes are depicted as not determined (ND). c) ELISpot screening of positively tested HLA-B*07:02-restricted peptides. Shown are numbers of IFNγ spot forming cells (SFC) for each tested donor minus the spot numbers of the negative control of the respective donor. Spot counts of >1000 were set to 1000 because of inaccurate spot count due to technical limitations. Positive evaluated spot counts are depicted in black, negative evaluated spot counts in grey. d) Comparison of IFNγ SFC in ex vivo ELISpots (black) and ELISpots with prior 12 day amplification (grey). Shown are exemplary results of five donors each for two B*08 epitopes (UL34 180-188 and UL26 61-69).

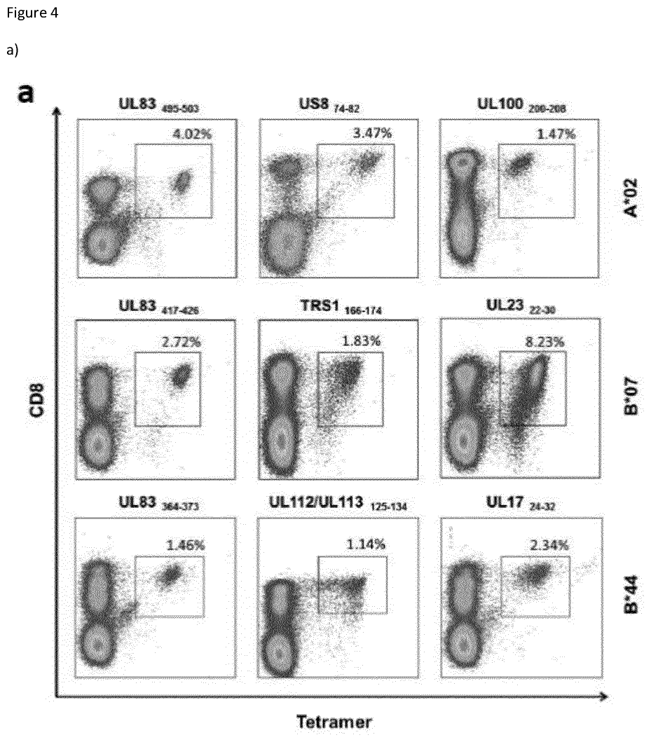

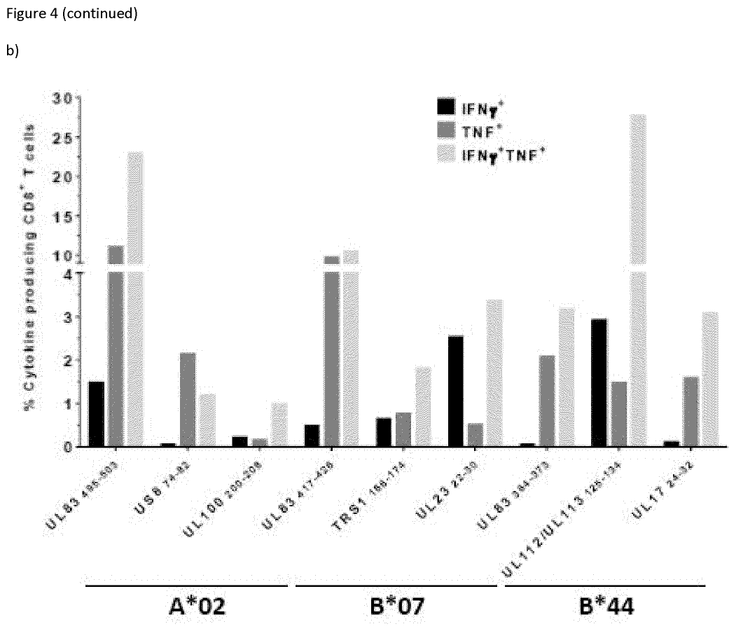

FIG. 4 shows the characterization of HCMV-specific memory T-cells. a) Representative tetramer staining after 12 day amplification in vitro of CD8+ T cells derived from HLA-matched healthy donors. Shown are results of three peptides per HLA. Novel HCMV ligands are compared to known pp65 epitopes (left column). b) Exemplary intracellular IFNγ and TNFα staining of healthy donors' PBMCs after 12 day amplification. Cells were stimulated with novel HCMV peptides or known pp65 epitopes. Bars represent percentage of CD8+ T cells producing IFNγ (black), TNFα (grey) or both (light grey). Three peptides per HLA restriction, tested in one HLA-matched donor, are shown.

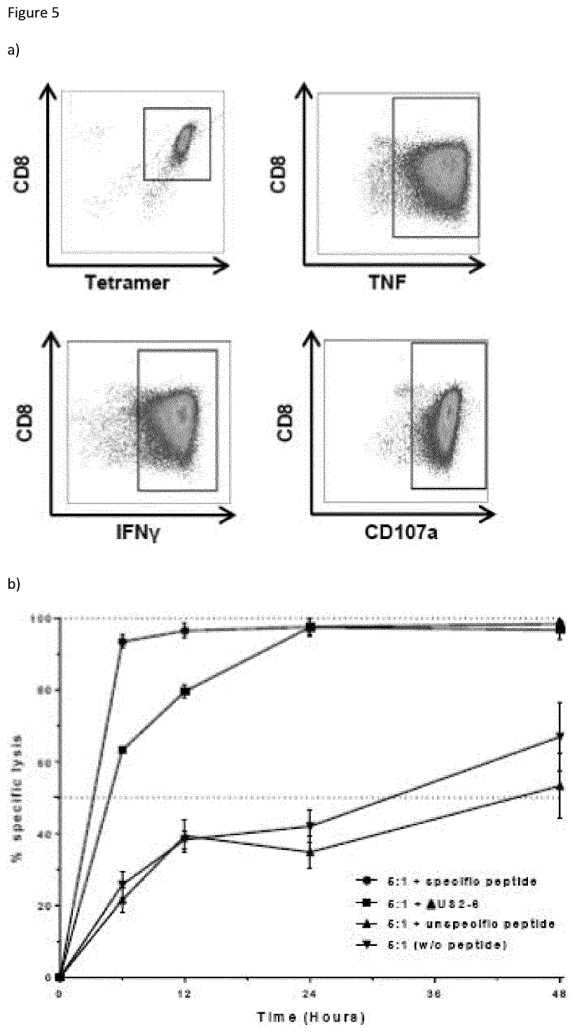

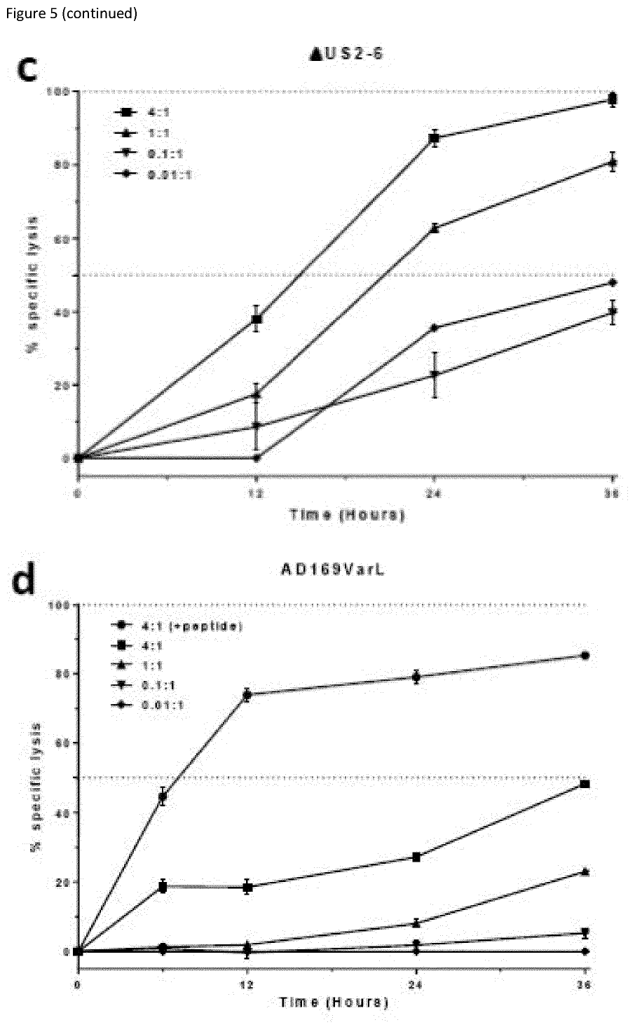

FIG. 5 shows the characterization and cytotoxicity of HCMV-specific CD8+ T-cell clones. a) Exemplary staining of a UL23 22-30-specific T-cell clone (B*07:02 restricted) with the respective tetramer and intracellular cytokine staining with TNF, IFNγ and the degranulation marker CD107a. b-d) Real-time cytotoxicity of different UL23 22-30-specific T-cell clones monitored by the xCELLigence system. 20,000 cells/well of infected or not infected MRC-5 cells were seeded into 96-well E-plates. After attachment of target cells, effector cells were added 48 h.p.i. at indicated E:T ratios (t0). Synthetic peptides were added to target cells one hour prior to effector cells (final concentration 1 μg/ml). Impedance was measured every 15 min and normalized to impedance of wells with medium only. The resulting dimensionless normalized cell index indicates the changes in impedance normalized to t0. Percentage of lysis was calculated in relation to cells without effector T cells. Experiments were performed in triplicates. b) MRC-5 cells were loaded with specific (UL23 22-30, RPWKPGQRV) (SEQ ID NO: 28) or unspecific (HIV Nef 128-137, TPGPGVRYPL) (SEQ ID NO: 103) peptide or infected with AD169 ΔUS2-6 (MOI 2) and incubated with effector cells in an E:T ratio of 5:1. Controls were MRC-5 cells without effector cells or without peptide. c) Comparison of specific lysis of ΔUS2-US6-infected MRC-5 cells with different E:T ratios. d) Specific lysis of AD169VarL wild type infected or peptide-loaded cells with indicated E:T ratios.

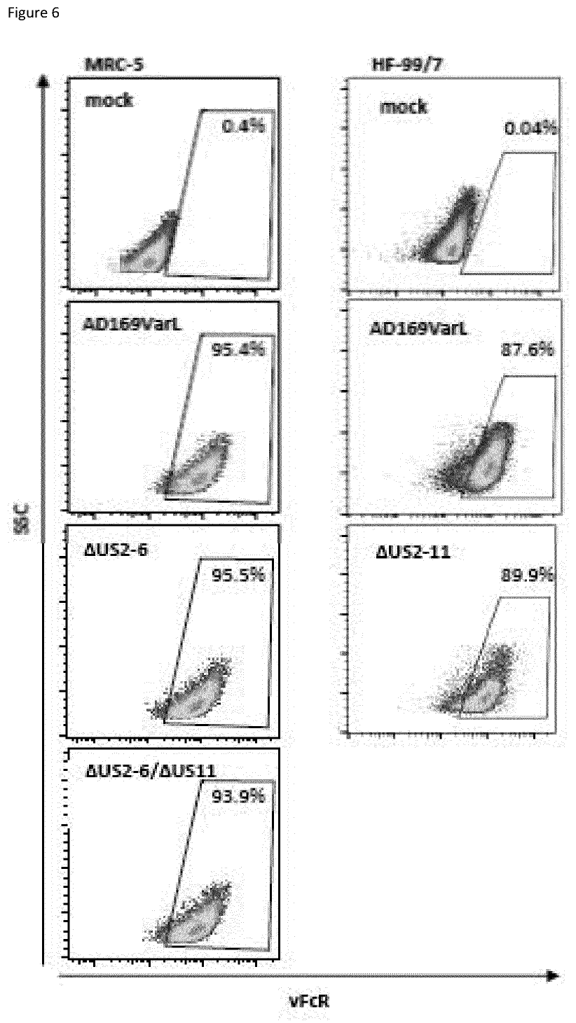

FIG. 6: The rate of infection was determined using the cells from FIG. 1. The cells were permeabilized and treated with Fc-FITC, which binds to the HCMV encoded Fc-receptors (vFcR). FITC levels were determined by flow cytometry.

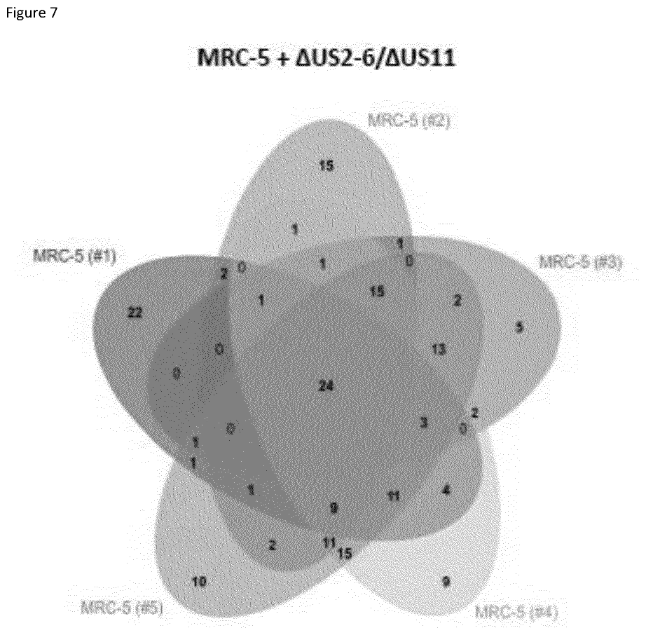

FIG. 7: Overlap of HCMV-derived HLA ligands between five independent HLA ligand elutions from MRC-5 cells infected with AD169 ΔUS2-6/AU1.

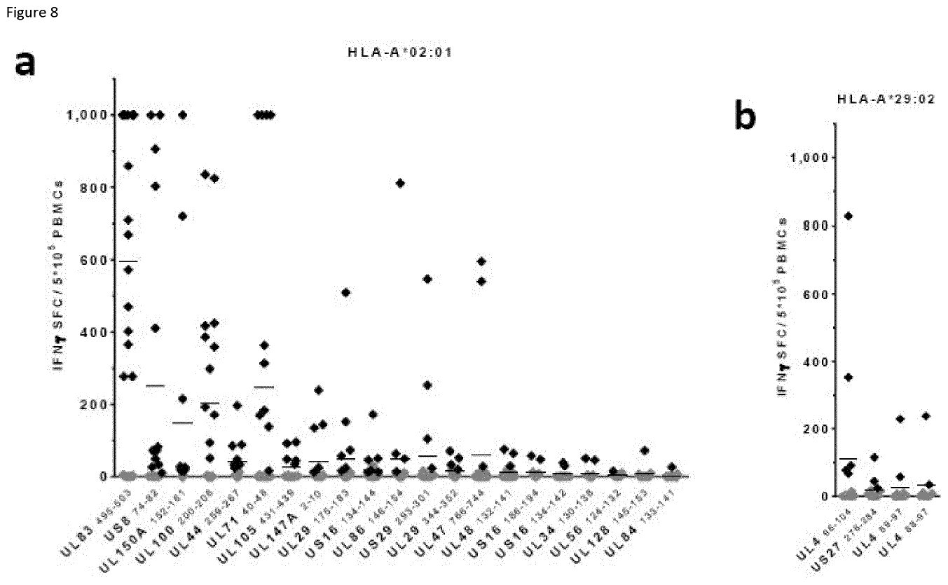

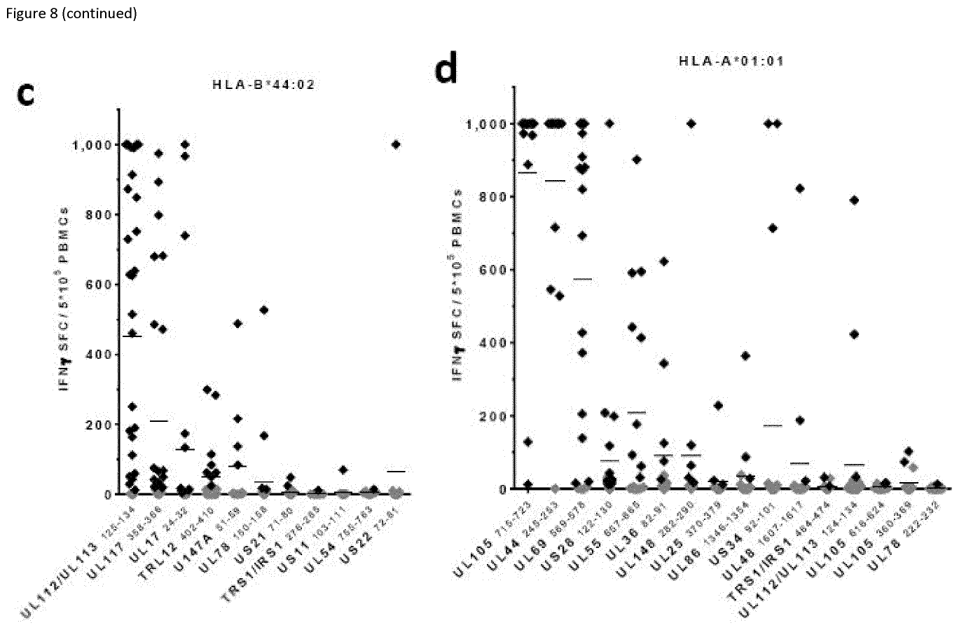

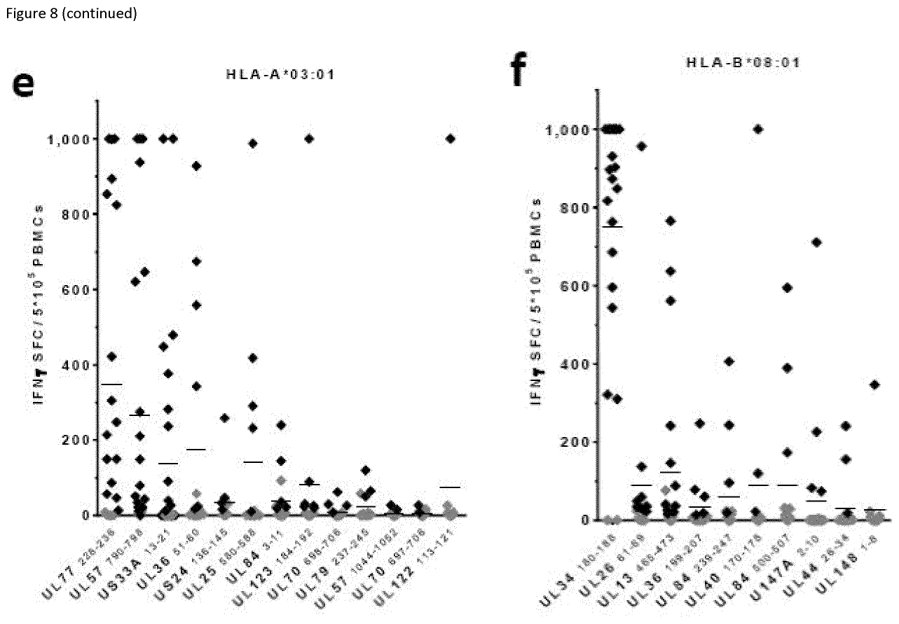

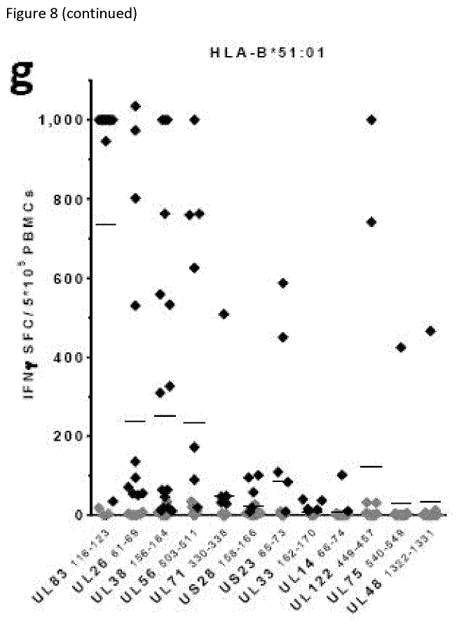

FIG. 8: ELISpot screening of positively tested peptides with HLA-A*02:01 (a), A*29:02 (b), B*44:02 (c), A*01:01 (d), A*03:01 (e), B*08:01 (f) and B*51:01 (g) restriction. Shown are numbers of IFNγ spot forming cells (SFC) for each tested donor minus the spot numbers of the negative control of the respective donor. Positive evaluated donors are depicted in black, negative tested donors in grey.

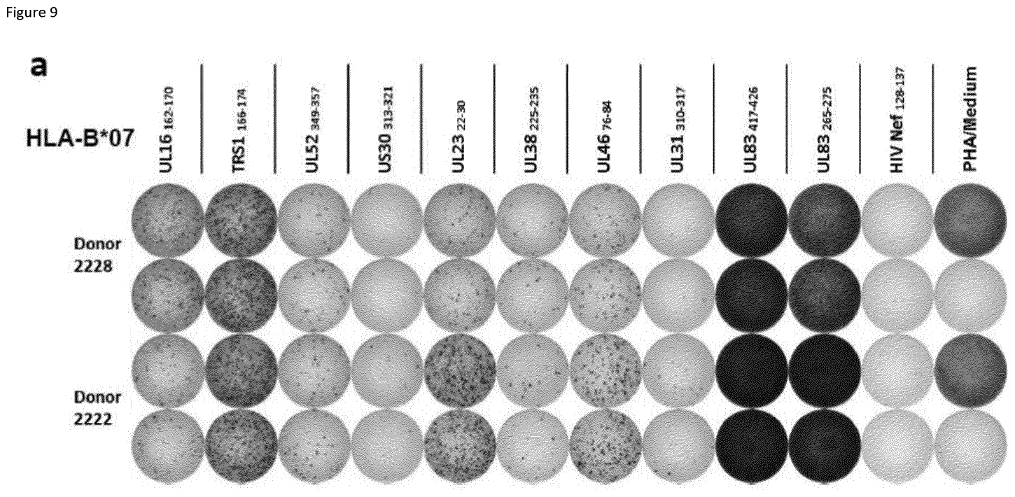

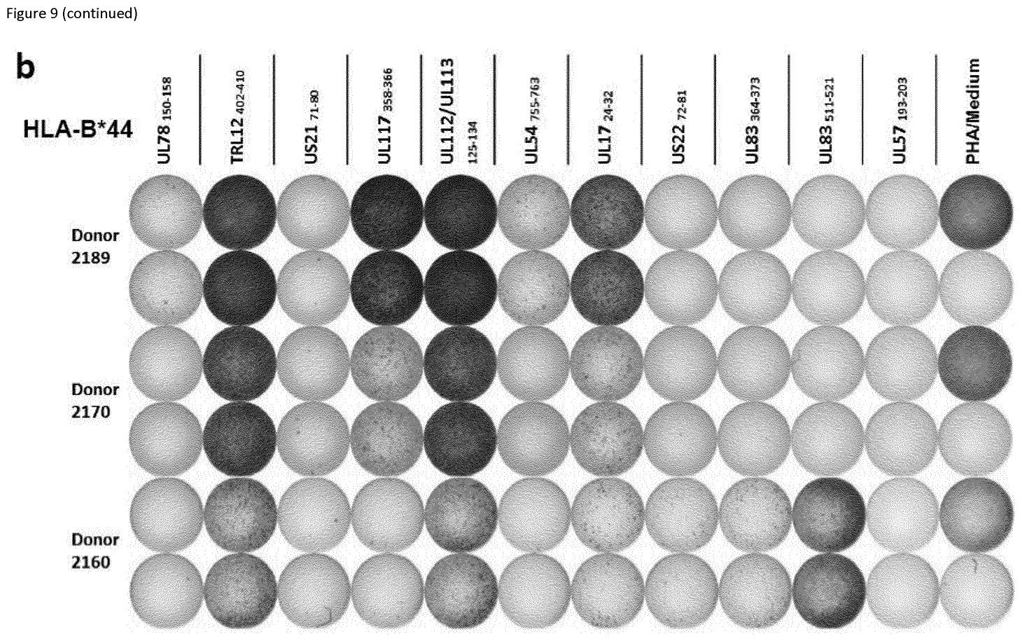

FIG. 9: Parallel recognition of multiple HCMV epitopes. Exemplary ELISpot results after 12 day amplification with HLA-B*07-restricted (a) and HLA-B*44-restricted (b) epitopes using PBMCs of two and three donors, respectively. PBMCs were stimulated with ten novel and already known epitopes (column 1-10). a) UL83 265-275 (RPHERNGFTVL, column 10) (SEQ ID NO: 102) is a previously identified epitope which was not contained in the here identified ligands. HIV Nef 128-137 (TPGPGVRYPL) (SEQ ID NO: 103) and medium served as negative controls, Phytohaemagglutinin (PHA) as positive control. b) UL83 364-373 (SEHPTFTSQY) (SEQ ID NO: 110) and UL83 511-521 (QEFFWDANDIY) (SEQ ID NO: 111) are already known epitopes that were not found as ligands in this study. UL57 193-203 (EEIPASDDVLF) (SEQ ID NO: 107) served as negative control.

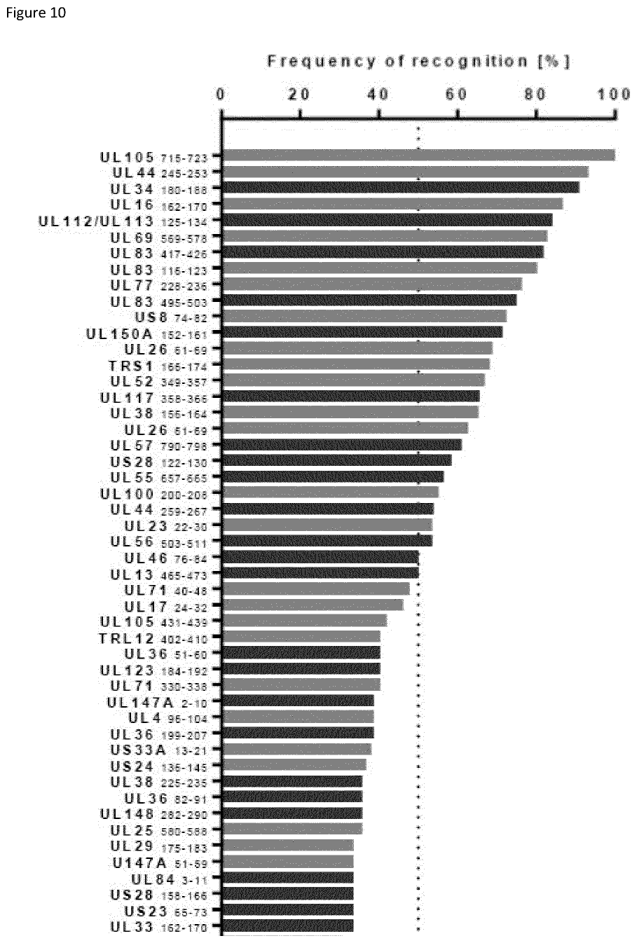

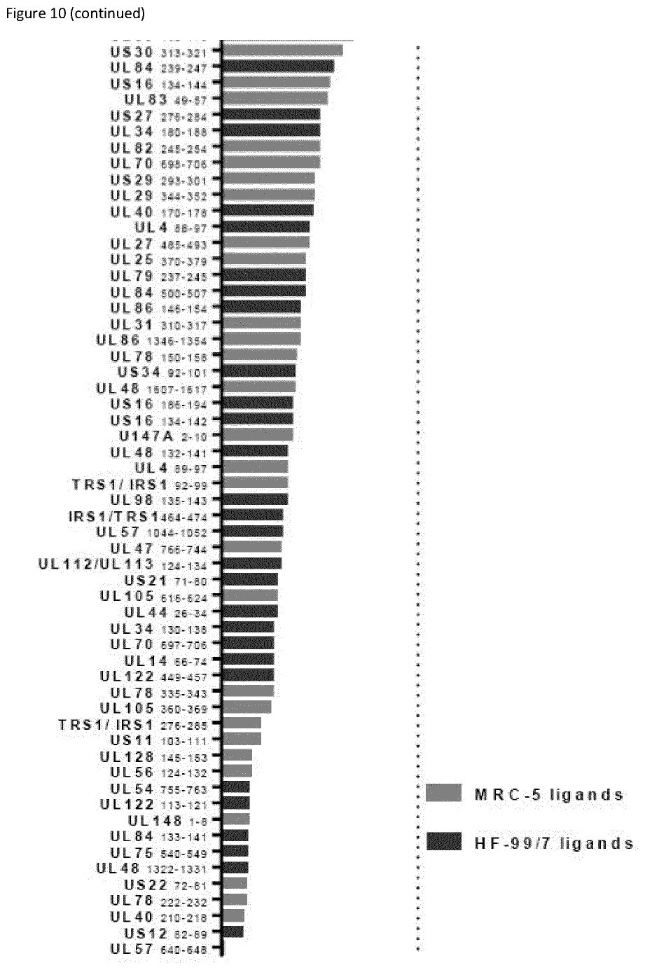

FIG. 10: Overview of frequencies of recognition by healthy donors for all identified HCMV epitopes. Dashed line indicates threshold for dominant epitopes.

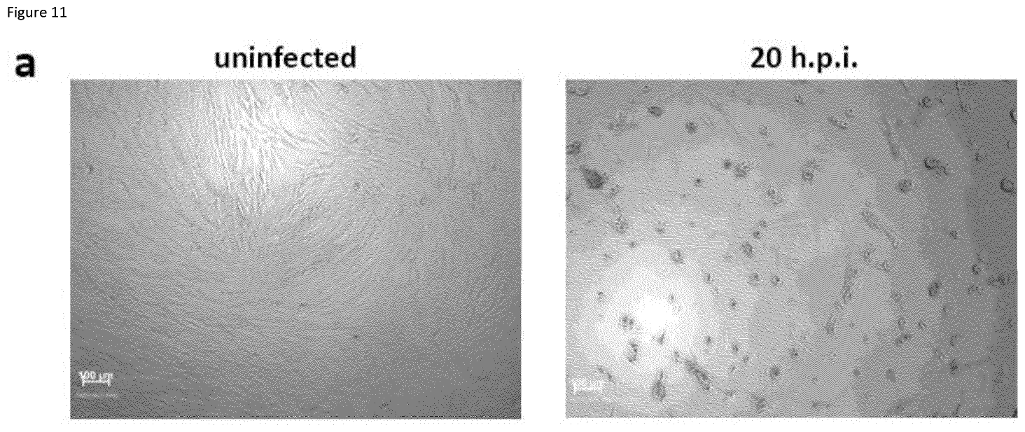

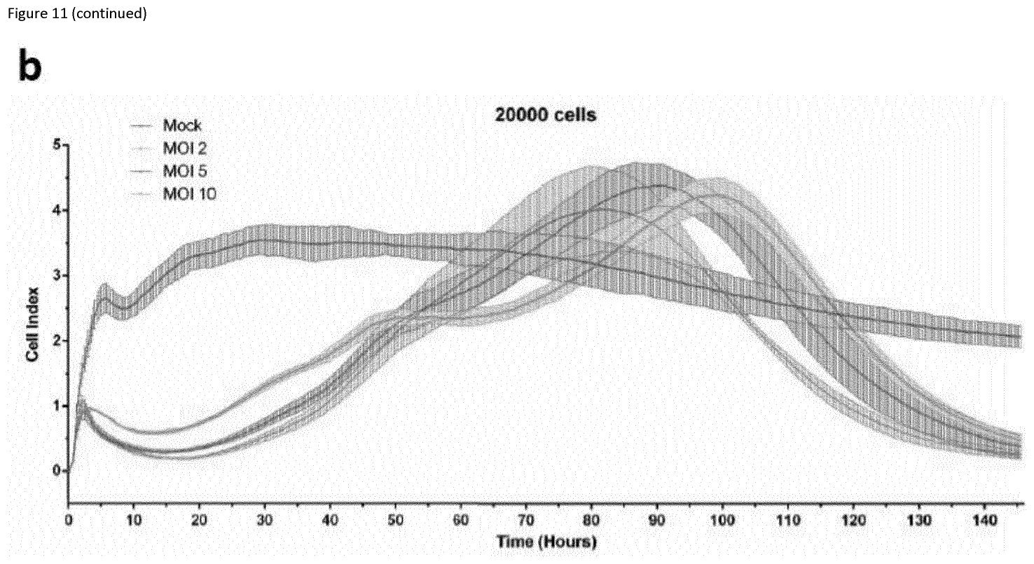

FIG. 11: Infection of MRC-5 cells with AD169 ΔUS2-6 for following cytotoxicity testing of peptide-specific T cell clones. a) Comparison of morphology of uninfected and infected (20 h.p.i., MOI 1) MRC-5 cells. b) Titration of MOIs in comparison with uninfected (mock) MRC-5 cells for the xCelligence system. 20,000 cells/well of infected or not infected MRC-5 cells were seeded into 96-well E-plates. Impedance was measured every 15 min and normalized to impedance of wells with medium only. The resulting dimensionless normalized cell index indicates the changes in impedance normalized to t0. Experiment was performed in triplicate.

Table 1 shows peptide epitopes of the invention, the source (underlying) protein, sequence, and other data relating to the peptides.

Table 2 shows data for preferred dominant epitopes of the invention.

### EXAMPLES

### Methods

### Cells and Viruses

MRC-5 fibroblasts (ECACC 05090501) and human foreskin fibroblasts (HF-99/7 ; donated as kind gift by Dieter Neumann-Haefelin and Valeria Kapper-Falcone, Freiburg) were grown in DMEM supplemented with 10% FCS, penicillin and streptomycin. The AD169VarL-based BAC mutants (51) were propagated on MRC-5 cells.

The recombinant HCMV mutants ΔUS2-6, ΔUS2-6/ΔUS11 and ΔUS2-11 were generated according to a previously published procedure (52) using the BAC-cloned AD169varL genome pAD169 (51) as parental BAC. Briefly, a PCR fragment was generated using the primers

KL-DeltaUS11-Kana1
(SEQ ID NO: 115)
CAAAAAGTCTGGTGAGTCGTTTCCGAGCGACT
CGAGATGCACTCCGCTTCAGTCTATATACCAG
TGAATTCGAGCTCGGTAC
and
KL-DeltaUS11-Kana2
(SEQ ID NO: 116)
TAAGACAGCCTTACAGCTTTTGAGTCTAGACA
GGGTAACAGCCTTCCCTTGTAAGACAGAGACC
ATGATTACGCCAAGCTCC

and the plasmid pSLFRTKn (53) as template DNA. The PCR fragment containing a kanamycin resistance gene was inserted 11 into the parental BAC by homologous recombination in E. coli. Correct mutagenesis was confirmed by Southern blot and PCR analysis. Recombinant HCMVs were reconstituted from HCMV BAC DNA by Superfect (Qiagen) transfection into permissive MRC-5 cells. Virus titers were determined by standard plaque assay.

### Flow Cytometry Analysis of Infected Cells

Cells were detached with accutase (Sigma) and stained with antibodies diluted in 3% FCS/PBS. Cells were washed in 3% FCS/PBS supplemented with DAPI and fixed in 3% paraformaldehyde. For intracellular staining of viral Fc-receptors cells were fixed and permeabilized using the BD Cytofix/Cytoperm™ Kit and stained with Fc-FITC (Rockland Immunochemicals Inc). Cells were measured with a BD FACSCanto™ II system (BD Biosciences) and acquired data was analyzed by FlowJo (v10.1, Tree Star Inc.). Analysis of HLA ligands by liquid chromatography-coupled tandem mass spectrometry (LC-MS/MS) Approximately 2-3×10⁸ cells (30 t175 flasks) of MRC-5 fibroblasts (A*02:01, A*29:02, B*07:02, B*44:02, C*05:01, and C*07:02) or human foreskin fibroblasts (HF-99/7) (A*01:01, A*03:01, B*08:01, B*51:01, C*01:02, C*07:01) were infected with an MOI of 4-7. At 48 h.p.i., the cells were collected by scraping and washed three times with PBS. The cell pellet was stored at −80° C. HLA-I ligands were isolated using standard immunoaffinity purification employing the pan-HLA class I-specific mAb W6/32 (54). HLA ligand extracts were analyzed as described previously (54). In brief, HLA ligand extracts were separated by reversed-phase liquid chromatography (nanoUHPLC, UltiMate 3000RSLCnano, Dionex) using a 75 μm×25 cm PepMap C18 column (Thermo Fisher Scientific). Linear gradients were applied ranging from 2.4% to 32% AcN over the course of 90 min in almost all analyses. In single experiments other methods, applying 195 or 300 min gradients on a 75 μm×50 cm PepMap column, were tested. Peptides eluted from MRC-5 cells were analyzed in an online coupled LTQ Orbitrap XL mass spectrometer (Thermo Fisher Scientific) using a top 5 collision induced fragmentation (CID) method generating ion trap MS/MS spectra. Extracts of HF-99/7 cells were analyzed in an online coupled LTQ Orbitrap Fusion Lumos mass spectrometer (Thermo Fisher Scientific) using a top speed CID method leading to orbitrap MS/MS spectra. Database search and filtering Data processing was performed as described previously (55). In brief, the Mascot search engine (Mascot 2.2.04; Matrix Science) (for ion trap fragment spectra) or the SEQUEST HT search engine (University of Washington) (for Orbitrap fragment spectra) (56) were used to search the human and HCMV proteome. Ion trap spectra were searched against a concatenated FASTA consisting of the Swiss-Prot reviewed human (September 2013; 20,279 sequences contained) and HCMV proteomes (April 2014; 400 sequences contained). Orbitrap spectra were searched against a FASTA consisting of the Swiss-Prot database of reviewed human proteins (March 2016; 20,270 sequences) and the HCMV proteome. The search combined data of technical replicates and was not restricted by enzymatic specificity. Precursor mass tolerance was set to 5 ppm, and fragment mass tolerance to 0.5 Da for ion trap spectra analyzed by Mascot and 0.02 Da for orbitrap spectra analyzed by SEQUEST HT, respectively. Oxidized methionine was allowed as a dynamic modification. FDR was estimated using the Percolator algorithm (57).

Peptide-12 spectrum matches were filtered for an FDR of 5%, search engine rank=1 and peptide lengths of 8-12 aa. For Mascot database searches the additional filter of Mascot Ion Scores≥20 were utilized. Peptide identifications were annotated to their respective HLA motifs using both SYFPEITHI (37), with a normalized score of ≥50%, and NetMHCv3.4 (58) for MRC-5 or Net MHCpan3.0 (59) for HF-99/7, applying IC 50≤500 nM percentile rank<2% (for NetMHCpan3.0) as cutoffs. Peptides fulfilling the cutoff in either or both prediction tools were designated as HLA ligands in this manuscript. In case of multiple possible annotations, the HLA allotype yielding the best rank/score was selected. Peptides were tested in donor samples of different restrict ions if the two algorithms resulted in inconsistent allotype annotations. Peptide and HLA: peptide monomer synthesis Synthetic peptides were produced by standard 9-fluorenylmethyloxycarbonyl/tert-butyl strategy using peptide synthesizers 433A (Applied Biosystems, Darmstadt, Germany), P11 (Activotec, Cambridge, UK) or Liberty Blue (CEM, Kamp-Lintfort, Germany). Purity was assessed by reversed phase HPLC (e2695, Waters, Eschborn, Germany) and identity affirmed by nano-UHPLC (UltiMate 3000 RSLCnano) coupled online to a hybrid mass spectrometer (LTQ Orbitrap XL, both Thermo Fisher). Lyophilized peptides were dissolved at 10 mg/ml in DMSO and diluted 1:10 in bidestilled H₂O. Frozen aliquots were further diluted in cell culture medium and sterile filtered if necessary. Synthetic peptides were used for validation of LC-MS/MS identifications as well as for functional experiments. Biotinylated recombinant HLA molecules and fluorescent HLA:peptide tetramers were produced as described previously (60-62). Target cell infection for cytotoxicity assays MRC-5 cells were cultured in DMEM (1×) (Life technologies) supplemented with 10% FCS, 100 U/ml penicillin and 100 μg/m1 streptomycin at 37° C. and 7.5% CO₂. For cytotoxicity assays MRC-5 cells were infected with an MOI of 2 and subsequently centrifuged for 30 min at 300 g. After resting for approximately 1 h cells were harvested by trypsination for 2 min at 37° C. and seeded in E-plates 96 (Roche) with 20,000 cells per well. T-cell culture blood samples were kindly provided by the Institute for Clinical and Experimental Transfusion Medicine at the University Hospital of Tubingen after obtaining written informed consent. Peripheral blood mononuclear cells (PBMCs) were isolated from healthy HCMV-seropositive blood donors by Ficoll-Hypaque density gradient centrifugation. Cells were frozen at −80° C. in FCS+10% DMSO. After thawing, cells were rested overnight prior to stimulation. Culture conditions were 7.5% CO₂ and 37° C. in humidified incubators in IMDM (PAA) supplemented with 5% heat-inactivated pooled human plasma (isolated from healthy blood donors), 100 U/ml penicillin, 100 μg/ml streptomycin, 25 μg/ml gentamicin (Life technologies) and 50 μM β-mercaptoethanol (Carl Roth). IFNγ-ELISpot assay The IFNγ-ELISpot assay was performed after 12 day stimulation as described previously (62) or directly ex vivo one day after thawing. Readout was performed according to manufacturers' recommendation and the cancer immunotherapy monitoring panel (63). PHA was used as positive control. The following 13 peptides, restricted to the respective HL A, served as negative controls: GSEELRSLY HIV POL 71-79 (A*01) (SEQ ID NO: 114), YLLPAIVHI HUMAN DDX5 148-156 (A*02) (SEQ ID NO: 104), RLRPGGKKK HIV GAG 20-28 (A*03) (SEQ ID NO: 105), TPGPGVRYPL HIV Nef 128-137 (B *07) (SEQ ID NO: 113), GGKKKYKL HIV GAG 24-31 (B*08) (SEQ ID NO: 106), EEIPASDDVLF HCMV DNBI 1095-1105 (B*44) (SEQ ID NO: 107), DPYKATSAV HUMAN MUC16 6326-6334 (B *51) (SEQ ID NO: 108). DMSO was used as a negative control for HLA-A*29. Blue spots specific for IFNγ-producing cells were automatically counted using an ImmunoSpot S5 analyzer (CTL) and ImmunoSpot Software. T-cell responses were considered to be positive when >10 spots/well were counted and mean spot count per well was at least 3-fold higher than the mean number of spots in negative control wells. Background staining due to excess cytokine and overlapping spots hamper the detection of reliable counts in wells of highly responsive donors. Therefore, spot counts of >1000 or “too numerous to count” were set to 1000. Analysis of T cells HLA tetramer staining of T cells was performed by incubation with 5 μg/ml tetramer diluted in tetramer staining buffer (2% FCS, 0.01% sodium azide and 2 mM EDTA in PBS) for 30 min at 4° C. Afterwards, T cells were stained with CD8-PerCP (Biolegend) for 20 min at 4° C. For ICS 0.5-1 Mio cells/well were stimulated with individual peptides (10 μg/ml) in presence of BrefeldinA (Sigma-Aldrich), GolgiStop (BD Biosciences) and anti-CD107a-FITC mAB (BD Biosciences) in 150 μl per well for 12-14 h. After incubation cells were washed and stained with anti-CD8-PerCP (Biolegend) and anti-CD4-APC (BD Biosciences) followed by fixation and permeabilization for further 20 min at 4° C. (Cytofix/Cytoperm, BD Biosciences). After washing with permwash buffer cytokines were stained with anti-TNFα-PacificBlue (Biologend) and anti-IFNγ-PE (BD Biosciences) for 20 min at 4° C. Flow cytometric measurements were performed on a FACSCanto II cytometer (BD Biosciences) with the DIVA software and analyzed using FlowJo Version 10. T-cell clones PBMCs of HLA-matched seropositive donors were stimulated with 1 μg/ml specific peptide one day after thawing and IL-2 (20 U/ml) (Novartis) on day 2 and 5. On day 14 HLA tetramer staining was performed and tetramer-positive CD8+ T cells were sorted in 96-well plates containing 1.5×10⁵ irradiated PBMCs (60-Gray, 1000 Elite Gammacell), 1.5×10⁴ irradiated LG2-EBV (200 Gray) (kind gift of Pierre van der Bruggen, Ludwig Institute for Cancer Research, Brussels, Belgium) as feeder cells, 150 U/ml IL-2 and 0.5 μg/ml PHA-L (Sigma-Aldrich) in 150 μl media per well. Sorting was performed using BD FACSJazz™ equipped with BD FACS™ Software. Five or ten tetramer-positive CD8+ T cells were sorted per well and incubated at 37° C. and 7.5% CO₂. After resting for one week cells were stimulated twice per week with 150 U/ml IL-2, freshly irradiated feeder cells (as described above) were added every second or third week together with 150 U/ml IL-2 and 1m/ml PHA-L (Roche).

### Real-Time Cytotoxicity Assay (XCelligence)

Real-time cytotoxicity assays were carried out as described previously (64). All experiments were performed in DMEM with 10% FCS and 1% PenStrep. Background values were determined using 50 μl medium per well. MRC-5 cells, infected or uninfected, were seeded in 96-well E-plates (Roche) at a concentration of 20,000 cells per well in 50 μl medium. Effector cells were added 48 h after target cells in 14 indicated E:T ratios. In case of peptide loading of MRC-5 cells, synthetic peptides (f.c. 1 μg/ml) were added to target cells one hour prior to effector cells. Cell attachment was monitored using the RTCA SP (Roche) instrument and the RTCA software Version 1.1 (Roche). Impedance measurements were performed every 15 minutes for up to 140 h. All experiments were performed in triplicates.

### Results

The epitope peptides as well as their characteristics as determined are depicted in the following tables 1 and 2:

TABLE 1
Peptide epitopes of the invention, the source (underlying)
protein, sequence, and other data relating to the peptides

| | | | | Actual | | | | | |
|---|---|---|---|---|---|---|---|---|---|
| | | | | HLA | | | | | |
| | | | | restric- | | | | | |
| | | | | tion | | % Posi- | | | |
| | | | | (identi- | | tive | | | |
| | | | | fied | | donors | % | | |
| | | | Pre- | using | Posi- | (after | Posi- | Intra- | |
| | Source | | dicted | tetramer | tive | 12 | tive | cellu- | |
| | protein | | HLA | staining | donors | days of | donors | lar | |
| Cell | and | Sequence/ | restric- | and | as | stimu- | (ex | cytokine | Tetramer- |
| line | position | SEQ ID NO: | tion | ICS) | tested | lation) | vivo) | staining | staining |
| MRC- | US8 74-82 | GVLDAVWRV | A*02:01 | A*02:01 | 13/18 | 72.2 | 50.0 | CD8 | Positive |
| 5 | | (SEQ ID NO: 1) | | | | | | | |
| MRC- | UL150A 152- | ALWDVALLEV | A*02:01 | A*02:01 | 10/14 | 71.4 | 25.0 | CD8 | Positive |
| 5 | 161 | (SEQ ID NO: 2) | | | | | | | |
| MRC- | UL100 200- | TLIVNLVEV | A*02:01 | A*02:01 | 11/20 | 55.0 | 25.0 | CD8 | Positive |
| 5 | 208 | (SEQ ID NO: 3) | | | | | | | |
| MRC- | UL44 259- | GLFAVENFL | A*02:01 | DR | 7/13 | 53.9 | 0.0 | CD4 | Nt |
| 5 | 267 | (SEQ ID NO: 4) | | | | | | | |
| MRC- | UL71 40-48 | FLDENFKQL | A*02:01 | DR | 10/21 | 47.6 | 37.5 | CD4 | Negative |
| 5 | | (SEQ ID NO: 5) | | | | | | | |
| MRC- | UL105 431- | RLFDLPVYC | A*02:01 | | 5/12 | 41.7 | Nt | Nt | Nt |
| 5 | 439 | (SEQ ID NO: 6) | | | | | | | |
| MRC- | UL29 175- | RLQPNVPLV | A*02:01 | | 6/18 | 33.3 | 0.0 | Nt | Nt |
| 5 | 183 | (SEQ ID NO: 7) | | | | | | | |
| MRC- | US16 134- | GLLAHIPALGV | A*02:01 | | 6/22 | 27.3 | 0.0 | Nt | Nt |
| 5 | 144 | (SEQ ID NO: 8) | | | | | | | |
| MRC- | US29 293- | ALSPSTSKV | A*02:01 | | 4/17 | 23.5 | Nt | Nt | Nt |
| 5 | 301 | (SEQ ID NO: 9) | | | | | | | |
| MRC- | UL29 344- | SLYEANPEL | A*02:01 | | 4/17 | 23.5 | Nt | Nt | Nt |
| 5 | 352 | (SEQ ID NO: 10) | | | | | | | |
| MRC- | UL86 146- | TILDKILNV | A*02:01 | | 4/20 | 20.0 | Nt | Nt | Nt |
| 5 | 154 | (SEQIDNO: 11) | | | | | | | |
| MRC- | US16 186- | TLINGVWVV | A*02:01 | | 2/11 | 18.2 | Nt | Nt | Nt |
| 5 | 194 | (SEQ ID NO: 12) | | | | | | | |
| MRC- | US16 134- | GLLAHIPAL | A*02:01 | | 2/11 | 18.2 | Nt | Nt | Nt |
| 5 | 142 | (SEQ ID NO: 13) | | | | | | | |
| MRC- | UL48 132- | ALYPEYIYTV | A*02:01 | | 3/18 | 16.7 | Nt | Nt | Nt |
| 5 | 141 | (SEQ ID NO: 14) | | | | | | | |
| MRC- | UL47 766- | GLNERLLSV | A*02:01 | | 3/20 | 15.0 | Nt | Nt | Nt |
| 5 | 744 | (SEQ ID NO: 15) | | | | | | | |
| MRC- | UL34 BO- | ALFNQLVFTA | A*02:01 | | 2/15 | 13.3 | Nt | Nt | Nt |
| 5 | 138 | (SEQ ID NO: 16) | | | | | | | |
| MRC- | UL56 124- | FTDNVRFSV | A*02:01 | | 1/13 | 7.8 | Nt | Nt | Nt |
| 5 | 132 | (SEQ ID NO: 17) | | | | | | | |
| MRC- | UL128 145- | GLDQYLESV | A*02:01 | | 1/13 | 7.8 | Nt | Nt | Nt |
| 5 | 153 | (SEQ ID NO: 18) | | | | | | | |
| MRC- | UL84 133- | ALLGRLYFI | A*02:01 | | 1/15 | 6.7 | Nt | Nt | Nt |
| 5 | 141 | (SEQ ID NO: 19) | | | | | | | |
| MRC- | UL4 96-104 | NYNEQHYRY | A*29:02 | | 5/13 | 38.5 | Nt | Nt | Nt |
| 5 | | (SEQ ID NO: 20) | | | | | | | |
| MRC- | US27 276- | LYVGQFLAY | A*29:02 | | 3/12 | 25.0 | Nt | Nt | Nt |
| 5 | 284 | (SEQ ID NO: 21) | | | | | | | |
| MRC- | UL4 88-97 | SFFPKLQGNY | A*29:02 | | 2/9 | 22.2 | Nt | Nt | Nt |
| 5 | | (SEQ ID NO: 22) | | | | | | | |
| MRC- | UL4 89-97 | FFPKLQGNY | A*29:02 | | 2/12 | 16.7 | Nt | Nt | Nt |
| 5 | | (SEQ ID NO: 23) | | | | | | | |
| MRC- | UL16 162- | YPRPPGSGL | B*07:02 | B*07:02 | 19/22 | 86.4 | 25.0 | Nega- | Positive |
| 5 | 170 | (SEQ ID NO: 24) | | | | | | tive | |
| MRC- | UL83 417- | TPRVTGGGAM | B*07:02 | B*07:02 | 31/38 | 81.6 | 100.0 | CD8 | Positive |
| 5 | 426 | (SEQ ID NO: 25) | | | | | | | |
| MRC- | TRS1 166- | SPRDAWIVL | B*07:02 | B*07:02 | 15/22 | 68.2 | 20.0 | CD8 | Positive |
| 5 | 174 | (SEQ ID NO: 26) | | | | | | | |
| MRC- | UL52 349- | SPSRDRFVQL | B*07:02 | B*07:02 | 14/21 | 66.7 | 33.3 | Nega- | Positive |
| 5 | 357 | (SEQ ID NO: 27) | | | | | | tive | |
| MRC- | UL23 22-30 | RPWKPGQRV | B*07:02 | B*07:02 | 15/28 | 53.6 | 66.7 | CD8 | Positive |
| 5 | | (SEQ ID NO: 28) | | | | | | | |
| MRC- | UL46 76-84 | SPRHLYISL | B*07:02 | B*07:02 | 11/22 | 50.0 | 0.0 | CD4/CD8 | Positive |
| 5 | | (SEQ ID NO: 29) | | | | | | | |
| MRC- | UL38 225- | IPMTFVDRDSL | B*07:02 | | 5/14 | 35.7 | Nt | Nt | Nt |
| 5 | 235 | (SEQ ID NO: 30) | | | | | | | |
| MRC- | US30 313- | RPFPSTHQL | B*07:02 | | 4/13 | 30.8 | 0.0 | Nt | Nt |
| 5 | 321 | (SEQ ID NO: 31) | | | | | | | |
| MRC- | UL83 49-57 | RVSQPSLIL | B*07:02 | | 4/15 | 26.7 | Nt | Nt | Nt |
| 5 | | (SEQ ID NO: 32) | | | | | | | |
| MRC- | UL82 245- | SPHPPTSVFL | B*07:02 | | 3/12 | 25.0 | Nt | Nt | Nt |
| 5 | 254 | (SEQ ID NO: 33) | | | | | | | |
| MRC- | UL27 485- | IPDYRSVSL | B*07:02 | | 4/18 | 22.2 | Nt | Nt | Nt |
| 5 | 493 | (SEQ ID NO: 34) | | | | | | | |
| MRC- | UL31 310- | APFGRVSV | B*07:02 | | 3/15 | 20.0 | Nt | Nt | Nt |
| 5 | 317 | (SEQ ID NO: 35) | | | | | | | |
| MRC- | TRS1/IRS1 | IPVERQAL | B*07:02 | | 2/12 | 16.7 | Nt | Nt | Nt |
| 5 | 92-99 | (SEQ ID NO: 36) | | | | | | | |
| MRC- | UL98 135- | APNYRQVEL | B*07:02 | | 2/12 | 16.7 | Nt | Nt | Nt |
| 5 | 143 | (SEQ ID NO: 37) | | | | | | | |
| MRC- | UL40 210- | LPNDHHYAL | B*07:02 | | 1/17 | 5.9 | Nt | Nt | Nt |
| 5 | 218 | (SEQ ID NO: 38) | | | | | | | |
| MRC- | US12 82-89 | APYLRDTL | B*07:02 | | 1/19 | 5.3 | Nt | Nt | Nt |
| 5 | | (SEQ ID NO: 39) | | | | | | | |
| MRC- | UL112/UL11 | SENGNLQVTY | B*44:02 | B*44:02 | 26/31 | 83.9 | 62.5 | CD8 | Positive |
| 5 | 3 125-134 | (SEQ ID NO: 40) | | | | | | | |
| MRC- | ULI 17 358- | HETGVYQMW | B*44:02 | B*44:02 | 17/26 | 65.4 | 62.5 | CD8 | Positive |
| 5 | 366 | (SEQ ID NO: 41) | | | | | | | |
| MRC- | UL17 24-32 | DEQVSKRSW | B*44:02 | B*44:02 | 11/24 | 45.8 | 12.5 | CD8 | Positive |
| 5 | | (SEQ ID NO: 42) | | | | | | | |
| MRC- | TRL12 402- | SESEFIVRY | B*44:02 | B*44:02 | 8/20 | 40.0 | 37.5 | CD8 | Positive |
| 5 | 410 | (SEQ ID NO: 43) | | | | | | | |
| MRC- | UL147A51- | EEQDYRALL | B*44:02 | | 4/12 | 33.3 | Nt | Nt | Nt |
| 5 | 59 | (SEQ ID NO: 44) | | | | | | | |
| MRC- | UL78 ISO- | RENAGVALY | B*44:02 | | 4/21 | 19.1 | Nt | Nt | Nt |
| 5 | 158 | (SEQ ID NO: 45) | | | | | | | |
| MRC- | US21 71-80 | AEPNFPKNVW | B*44:02 | | 2/14 | 14.3 | Nt | Nt | Nt |
| 5 | | (SEQ ID NO: 46) | | | | | | | |
| MRC- | TRSl/IRS1 | EEATALGREL | B*44:02 | | 1/10 | 10.0 | Nt | Nt | Nt |
| 5 | 276-285 | (SEQ ID NO: 47) | | | | | | | |
| MRC- | US 11 103- | SESLVAKRY | B*44:02 | | 1/10 | 10.0 | Nt | Nt | Nt |
| 5 | 111 | (SEQ ID NO: 48) | | | | | | | |
| MRC- | UL54 755- | LENGVTHRF | B*44:02 | | 1/14 | 7.1 | Nt | Nt | Nt |
| 5 | 763 | (SEQ ID NO: 49) | | | | | | | |
| MRC- | US22 72-81 | REQAAIPQIY | B*44:02 | | 1/16 | 6.3 | Nt | Nt | Nt |
| 5 | | (SEQ ID NO: 50) | | | | | | | |
| HF- | UL105 715- | YADPFFLKY | A*01:01 | A*01:01 | 15/15 | 100.0 | 90.9 | CD8 | Positive |
| 99/7 | 723 | (SEQ ID NO: 51) | | | | | | | |
| HF- | UL44 245- | VTEHDTLLY | A*01:01 | A*01:01 | 13/14 | 92.9 | 100.0 | Nt | Nt |
| 99/7 | 253 | (SEQ ID NO: 52) | | | | | | | |
| HF- | UL69 569- | RTDPATLTAY | A*01:01 | A*01:01 | 19/23 | 82.6 | 66.7 | CD8 | Positive |
| 99/7 | 578 | (SEQ ID NO: 53) | | | | | | | |
| HF- | US28 122- | ITEIALDRY | A*01:01 | A*01:01 | 14/24 | 58.3 | 14.3 | CD8 | Positive |
| 99/7 | 130 | (SEQ ID NO: 54) | | | | | | | |
| HF- | UL55 657- | NTDFRVLELY | A*01:01 | A*01:01 | 9/16 | 56.3 | 0.0 | CD8 | Positive |
| 99/7 | 665 | (SEQ ID NO: 55) | | | | | | | |
| HF- | UL36 82-91 | FVEGPGFMRY | A*01:01 | | 5/14 | 35.7 | Nt | Nt | Nt |
| 99/7 | | (SEQ ID NO: 56) | | | | | | | |
| HF- | UL148 282- | SLDRFIVQY | A*01:01 | DR | 5/14 | 35.7 | Nt | CD4 | Nt |
| 99/7 | 290 | (SEQ ID NO: 57) | | | | | | | |
| HF- | UL25 370- | YTSRGALYLY | A*01:01 | | 3/14 | 21.4 | Nt | Nt | Nt |
| 99/7 | 379 | (SEQ ID NO: 58) | | | | | | | |
| HF- | UL86 1346- | TSETHFGNY | A*01:01 | | 3/15 | 20.0 | Nt | Nt | Nt |
| 99/7 | 1354 | (SEQ ID NO: 59) | | | | | | | |
| HF- | US34 92-101 | GSDALPAGLY | A*01:01 | | 3/16 | 18.8 | Nt | Nt | Nt |
| 99/7 | | (SEQ ID NO: 60) | | | | | | | |
| HF- | UL48 1607- | VTDYGNVAFK | A*01:01 | | 3/16 | 18.8 | Nt | Nt | Nt |
| 99/7 | 1617 | Y | | | | | | | |
| | | (SEQ ID NO: 61) | | | | | | | |
| HF- | IRSl/TRSI | LLDELGAVFG | A*01:01 | | 2/13 | 15.4 | Nt | Nt | Nt |
| 99/7 | 464-474 | Y | | | | | | | |
| | | (SEQ ID NO: 62) | | | | | | | |
| HF- | UL112/UL113 | ISENGNLQVTY | A*01:01 | | 3/20 | 15.0 | Nt | Nt | Nt |
| 99/7 | 124-134 | (SEQ ID NO: 63) | | | | | | | |
| HF- | UL105 616- | VTDPEHLMM | A*01:01 | | 2/14 | 14.3 | Nt | Nt | Nt |
| 99/7 | 624 | (SEQ ID NO: 64) | | | | | | | |
| HF- | UL105 360- | DLDFGDLLKY | A*01:01 | | 2/16 | 12.5 | Nt | Nt | Nt |
| 99/7 | 369 | (SEQ ID NO: 65) | | | | | | | |
| HF- | UL78 222- | YSDRRDHVWS | A*01:01 | | 1/16 | 6.3 | Nt | Nt | Nt |
| 99/7 | 232 | Y | | | | | | | |
| | | (SEQ ID NO: 66) | | | | | | | |
| HF- | UL77 228- | GLYTQPRWK | A*03:01 | A*03:01 | 16/21 | 76.2 | 50.0 | CD8 | Positive |
| 99/7 | 236 | (SEQ ID NO: 67) | | | | | | | |
| HF- | UL57 790- | RVKNRPIYR | A*03:01 | A*03:01 | 14/23 | 60.9 | 33.3 | CD8 | Positive |
| 99/7 | 798 | (SEQ ID NO: 68) | | | | | | | |
| HF- | UL36 51-60 | RSALGPFVGK | A*03:01 | A*03:01 | 6/15 | 40.0 | Nt | CD8 | Positive |
| 99/7 | | (SEQ ID NO: 69) | | | | | | | |
| HF- | UL123 184- | KLGGALQAK | A*03:01 | | 6/15 | 40.0 | Nt | Nt | Nt |
| 99/7 | 192 | (SEQ ID NO: 70) | | | | | | | |
| HF- | US33A 13-21 | KLGYRPHAK | A*03:01 | A*03:01 | 11/29 | 37.9 | Nt | CD8 | Positive |
| 99/7 | | (SEQ ID NO: 71) | | | | | | | |
| HF- | US24 136- | RVYAYDTREK | A*03:01 | | 4/11 | 36.4 | Nt | Nt | Nt |
| 99/7 | 145 | (SEQ ID NO: 72) | | | | | | | |
| HF- | UL25 580- | GVSSVTLLK | A*03:01 | | 5/14 | 35.7 | Nt | Nt | Nt |
| 99/7 | 588 | (SEQ ID NO: 73) | | | | | | | |
| HF- | UL84 3-11 | RVDPNLRNR | A*03:01 | | 5/15 | 33.3 | Nt | Nt | Nt |
| 99/7 | | (SEQ ID NO: 74) | | | | | | | |
| HF- | UL70 698- | SVRLPYMYK | A*03:01 | | 4/16 | 25.0 | Nt | Nt | Nt |
| 99/7 | 706 | (SEQ ID NO: 75) | | | | | | | |
| HF- | UL79 237- | RTFAGTLSR | A*03:01 | | 3/14 | 21.4 | Nt | Nt | Nt |
| 99/7 | 245 | (SEQ ID NO: 76) | | | | | | | |
| HF- | UL57 1044- | RLADVLIKR | A*03:01 | | 2/13 | 15.4 | Nt | Nt | Nt |
| 99/7 | 1052 | (SEQ ID NO: 77) | | | | | | | |
| HF- | UL70 697- | RSVRLPYMYK | A*03:01 | | 2/15 | 13.3 | Nt | Nt | Nt |
| 99/7 | 706 | (SEQ ID NO: 78) | | | | | | | |
| HF- | UL122 113- | SVSSAPLNK | A*03:01 | | 1/14 | 7.1 | Nt | Nt | Nt |
| 99/7 | 121 | (SEQ ID NO: 79) | | | | | | | |
| HF- | UL13 465- | YLVRRPMTI | B*08:01 | B*08:01 | 11/22 | 50.0 | 33.3 | Nega- | Positive |
| 99/7 | 473 | (SEQ ID NO: 80) | | | | | | tive | |
| HF- | UL36 199- | VMKFKETSF | B*08:01 | | 5/13 | 38.5 | Nt | Nt | Nt |
| 99/7 | 207 | (SEQ ID NO: 81) | | | | | | | |
| HF- | UL84 239- | TPLLKRLPL | B*08:01 | | 4/14 | 28.6 | Nt | Nt | Nt |
| 99/7 | 247 | (SEQ ID NO: 82) | | | | | | | |
| HF- | UL40 170- | HLKLRPATF | B*08:01 | | 3/13 | 23.1 | Nt | Nt | Nt |
| 99/7 | 178 | (SEQ ID NO: 83) | | | | | | | |
| HF- | UL84 500- | FISSKHTL | B*08:01 | | 3/14 | 21.4 | Nt | Nt | Nt |
| 99/7 | 507 | (SEQ ID NO: 84) | | | | | | | |
| HF- | UL44 26-34 | QLRSVIRAL | B*08:01 | | 2/14 | 14.3 | Nt | Nt | Nt |
| 99/7 | | (SEQ ID NO: 85) | | | | | | | |
| HF- | UL148 1-8 | MLRLLFTL | B*08:01 | B*08:01 | 1/14 | 7.1 | Nt | Nt | Nt |
| 99/7 | | (SEQ ID NO: 86) | | | | | | | |
| HF- | UL83 116- | LPLKMLNI | B*51:01 | B*51:01 | 12/15 | 80.0 | 87.5 | CD8 | Positive |
| 99/7 | 123 | (SEQ ID NO: 87) | | | | | | | |
| HF- | UL38 156- | FPVEVRSHV | B*51:01 | B*51:01 | 15/23 | 65.2 | 0.0 | CD8 | Positive |
| 99/7 | 164 | (SEQ ID NO: 88) | | | | | | | |
| HF- | UL56 503- | DARSRIHNV | B*51:01 | B*51:01 | 8/15 | 53.3 | Nt | CD8 | Positive |
| 99/7 | 511 | (SEQ ID NO: 89) | | | | | | | |
| HF- | UL71 330- | IPPPQIPFV | B*51:01 | | 6/15 | 40.0 | Nt | Nt | Nt |
| 99/7 | 338 | (SEQ ID NO: 90) | | | | | | | |
| HF- | US28 158- | IAIPHFMVV | B*51:01 | | 5/15 | 33.3 | Nt | Nt | Nt |
| 99/7 | 166 | (SEQ ID NO: 91) | | | | | | | |
| HF- | US23 65-73 | IPHNWFLQV | B*51:01 | | 5/15 | 33.3 | Nt | Nt | Nt |
| 99/7 | | (SEQ ID NO: 92) | | | | | | | |
| HF- | UL33 162- | VPAAVYTTV | B*51:01 | | 5/15 | 33.3 | Nt | Nt | Nt |
| 99/7 | 170 | (SEQ ID NO: 93) | | | | | | | |
| HF- | ULM 66-74 | FPAHDWPEV | B*51:01 | | 2/15 | 13.3 | Nt | Nt | Nt |
| 99/7 | | (SEQ ID NO: 94) | | | | | | | |
| HF- | UL122 449- | MPVTHPPEV | B*51:01 | | 2/15 | 13.3 | Nt | Nt | Nt |
| 99/7 | 457 | (SEQ ID NO: 95) | | | | | | | |
| HF- | UL75 540- | FPDATVPATV | B*51:01 | | 1/15 | 6.7 | Nt | Nt | Nt |
| 99/7 | 549 | (SEQ ID NO: 96) | | | | | | | |
| HF- | UL48 1322- | LPYLSAERTV | B*51:01 | | 1/15 | 6.7 | Nt | Nt | Nt |
| 99/7 | 1331 | (SEQ ID NO: 97) | | | | | | | |
| MRC- | UL147A 2-10 | SLFYRAVAL | A*02:01 | | 5/13 | 38.5 | 12.5 | Nt | Nt |
| 5 | | (SEQ ID NO: 98) | B*08:01 | | 4/22 | 18.2 | Nt | Nt | Nt |
| HF- | | | | | | | | | |
| 99/7 | | | | | | | | | |
| HF- | UL26 61-69 | LPYPRGYTL | B*08:01 | B*08:01/ | 11/16 | 68.8 | 16.7 | CD8 | positive |
| 99/7 | | (SEQ ID NO: 99) | B*51:01 | B51*:01 | 10/16 | 62.5 | 33.3 | CD8 | positive |
| HF- | | | | B*08:01/ | | | | | |
| 99/7 | | | | B*51:01 | | | | | |
| HF- | UL34 180- | LPHERHREL | B*08:01 | B*08:01 | 20/22 | 90.9 | 85.7 | CD8 | positive |
| 99/7 | 188 | (SEQ ID NO: | B*07:02 | | 3/12 | 25.0 | Nt | Nt | Nt |
| MRC- | | 100) | | | | | | | |
| 5 | | | | | | | | | |

TABLE 2
Summary of dominant epitopes. Ex vivo ELISpots were performed using donors
that were positively tested in ELISpots with 12d stimulation.
Abbreviations: 12d stim, 12-day amplification with IL-2 in vitro; nt, not tested.

| | | | | ELISpot | | | |
|---|---|---|---|---|---|---|---|
| | | | actual | response | ELISpot | Intra- | |
| | | | HLA | rate | response | cellu- | |
| | Sequence | Tested | restric- | (12 d | rate | lar | Tetramer |
| Protein | SEQ ID NO: | HLA | tion | stim.) | (ex vivo)† | staining | staining |
| UL83 495-503 | NLVPMVATV* | A*02:01 | A*02:01 | 75.0 | 100.0 | CD8 | positive |
| | SEQ ID NO: 101 | | | | | | |
| US8 74-82 | GVLDAVWRV | A*02:01 | A*02:01 | 72.2 | 50.0 | CD8 | positive |
| | SEQ ID NO: 1 | | | | | | |
| UL150A 152- | ALWDVALLEV | A*02:01 | A*02:01 | 71.4 | 25.0 | CD8 | positive |
| 161 | SEQ ID NO: 2 | | | | | | |
| UL100 200-208 | TLIVNLVEV | A*02:01 | A*02:01 | 55.0 | 25.0 | CD8 | positive |
| | SEQ ID NO: 3 | | | | | | |
| UL44 259-267 | GLFA VENFL | A*02:01 | Class II | 53.9 | 0.0 | CD4 | not tested |
| | SEQ ID NO: 4 | | | | | | |
| UL16 162-170 | YPRPPGSGL* | B*07:02 | B*07:02 | 86.4 | 25.0 | nega- | positive |
| | SEQ ID NO: 24 | | | | | tive | |
| UL83 417-426 | TPRVTGGGAM* | B*07:02 | B*07:02 | 81.6 | 100.0 | CD8 | positive |
| | SEQ ID NO: 25 | | | | | | |
| TRS1 166-174 | SPRDAWIVL | B*07:02 | B*07:02 | 68.2 | 20.0 | CD8 | positive |
| | SEQ ID NO: 26 | | | | | | |
| UL52 349-357 | SPSRDRFVQL | B*07:02 | B*07:02 | 66.7 | 33.3 | nega- | positive |
| | SEQ ID NO: 27 | | | | | tive | |
| UL23 22-30 | RPWKPGQRV | B*07:02 | B*07:02 | 53.6 | 66.7 | CD8 | positive |
| | SEQ ID NO: 28 | | | | | | |
| UL46 76-84 | SPRHLYISL | B*07:02 | B*07:02 | 50.0 | 0.0 | CD4/ | positive |
| | SEQ ID NO: 29 | | | | | CD8 | |
| UL112/UL113 | SENGNLQVTY | B*44:02 | B*44:02 | 83.9 | 62.5 | CD8 | positive |
| 125-134 | SEQ ID NO: 40 | | | | | | |
| UL117 358-366 | HETGVYQMW | B*44:02 | B*44:02 | 65.4 | 62.5 | CD8 | positive |
| | SEQ ID NO: 41 | | | | | | |
| UL105 715-723 | YADPFFLKY* | A*01:01 | A*01:01 | 100.0 | 90.9 | CD8 | positive |
| | SEQ ID NO: 51 | | | | | | |
| UL44 245-253 | VTEHDTLLY* | A*01:01 | A*01:01 | 92.9 | 100.0 | CD8 | positive |
| | SEQ ID NO: 52 | | | | | | |
| UL69 569-578 | RTDPATLTAY | A*01:01 | A*01:01 | 82.6 | 66.7 | CD8 | positive |
| | SEQ ID NO: 53 | | | | | | |
| US28 122-130 | ITEIALDRY | A*01:01 | A*01:01 | 58.3 | 14.3 | CD8 | positive |
| | SEQ ID NO: 54 | | | | | | |
| UL55 657-665 | NTDFRVLELY | A*01:01 | A*01:01 | 56.3 | 0.0 | CD8 | positive |
| | SEQ ID NO: 55 | | | | | | |
| UL77 228-236 | GLYTQPRWK | A*03:01 | A*03:01 | 76.2 | 50.0 | CD8 | positive |
| | SEQ ID NO: 67 | | | | | | |
| UL57 790-798 | RVKNRPIYR | A*03:01 | A*03:01 | 60.9 | 33.3 | CD8 | positive |
| | SEQ ID NO: 68 | | | | | | |
| UL34 180-188 | LPHERHREL | B*08:01 | B*08:01 | 90.9 | 85.7 | CD8 | positive |
| | SEQ ID NO: 100 | | | | | | |
| UL26 61-69 | LPYPRGYTL | B*08:01 | B*08:01/ | 68.8 | 16.7 | CD8 | positive |
| | SEQ ID NO: 99 | | 51:01 | | | | |
| UL13 465-473 | YLVRRPMTI | B*08:01 | B*08:01 | 50.0 | 33.3 | nega- | positive |
| | SEQ ID NO: 80 | | | | | tive | |
| UL83 116-123 | LPLKMLNI* | B*51:01 | B*51:01 | 80.0 | 87.5 | CD8 | positive |
| | SEQ ID NO: 87 | | | | | | |
| UL38 156-164 | FPVEVRSHV | B*51:01 | B*51:01 | 65.2 | 0.0 | CD8 | positive |
| | SEQ ID NO: 88 | | | | | | |
| UL26 61-69 | LPYPRGYTL | B*51:01 | B*08:01/ | 62.5 | 33.3 | CD8 | positive |
| | SEQ ID NO: 99 | | 51:01 | | | | |
| UL56 503-511 | DARSRIHNV | B*51:01 | B*51:01 | 53.3 | 20.0 | CD8 | positive |
| | SEQ ID NO: 89 | | | | | | |

Deletion of HCMV encoded immunoevasins rescues HLA-I expression of infected cells So far, attempts to isolate naturally presented HCMV derived HLA-I ligands have not been successful. HCMV encodes for several immunoevasins targeting HLA-I at various stages of the antigen presentation pathway. Therefore, the inventors speculated that deletion of genes involved in HLA-I regulation would enable the identification of virally encoded HLA-I ligands. The inventors constructed AD169VarL (with partial ULb′ region (35)) deletion mutants lacking the genes US2-6 (ΔUS2-6), US2-6+US11 (ΔUS2-6/US11) and US2-11 (ΔUS2-11). To measure the level of HLA-I rescue due to lack of specific immunoevasins, the inventors infected two different fibroblast cell cultures expressing HLA-I types of interest: MRC-5 (HLA-A*02:01, -A*29:02, -B*07:02, -B*44:02, -C*05:01, and -C*07:02) and HFF-99/7 (HLA-A*01:01, A*03:01, B*08:01, B*51:01, C*01:02, and C*07:01). The rate of infection was determined using Fc-FITC, which binds to the HC MV encoded Fc-receptors (vFcR) (FIG. 6). At 48 h post-infection (h.p.i.) the HLA-I cell surface level was determined by flow cytometry using the pan-HLA-I antibody W6/32 (FIG. 1). Interestingly, HLA-I downregulation by AD 169VarL wild-type virus varied strongly between fibroblasts. Since in MRC-5 cells HLA-B*44:02 is expressed at very low level in mock treated cells, but is induced strongly in HCMV infected cells, this molecule could be the reason for the apparent low level of reduction by AD169VarL (compared to mock treated cells). As expected, infection with HCMV mutant viruses lacking HLA-I immunoevasins showed a robust rescue of HLA-I at the cell surface and the inventors next proceeded with detailed HLA-I ligandome analysis using the virus mutants. Direct identification of HCMV-derived HLA-I ligands by LC-MS/MS First, for direct identification of processed and presented HCMV-derived HLA-I ligands the inventors performed mass spectrometric analysis of immunoaffinity-purified peptide extracts isolated from MRC-5 cells. At 48 h.p.i. , HLA-I ligands isolated from cells infected with AD169VarL (n=1 sample), ΔUS2-6 (n=3 samples), ΔUS2-6/ΔUS11 (n=5 samples), and mock controls (n=1 sample) were exhaustively analyzed in five to seven LC-MS/MS runs per sample. These MS analyses revealed 816 to 2,714 unique HLA ligands per sample (FIG. 2a). As expected, only 3/816 (0.4%) of HLA ligands, eluted from MRC-5 cells infected with AD169VarL wild-type virus, were derived from HCMV, while infection with the deletion viruses resulted in substantially increased viral peptide identification rates and numbers. In MRC-5 cells infected with the mutant viruses ΔUS2-6 and ΔUS2-6/ΔUS11 a total of 79 and 181 HCMV-derived HLA ligands were identified, respectively, resulting in a total number of 194 unique viral peptides. Overlap analysis revealed 66/194 viral peptides to be presented on MRC-5 after infection with both deletion viruses (FIG. 2b). Interestingly, 13/79 (17%) and 114/181 (63%) viral peptides of ΔUS2-6 and ΔUS2-6/ΔUS11 infected cells, respectively, were unique. This demonstrates that the HLA-I immunoevasins not only affect the quantity, but also the quality of HLA-I antigen processing and presentation. Therefore, the use of varying HCMV deletion mutants can result in a higher variability of identified HCMV-derived peptide species. Furthermore, the inventors isolated the HLA-presented peptides from several biological replicates for each infection to maximize the number of identified HCMV-derived peptides. Thereby, the inventors were able to identify between 37 and 63 (mean: 51) unique viral HLA ligands on cells infected with ΔUS2-6, corresponding to 2.4-3.0% (mean: 2.8%) of total HLA ligand identifications. Overlap analysis of viral ligands identified in the three independent HLA precipitations revealed 31/79 (39%) of peptides to be uniquely identified in a single experiment, while 61% showed reproducible identification in at least two out of three experiments (FIG. 2c). On cells infected with ΔUS2-6/ΔUS11 even higher proportions of viral ligands were identified, resulting in 79-119 (mean: 93) unique viral HLA ligands corresponding to 3.2-4.5% (mean: 3.9%) of total HLA ligands. Here, a similar degree of reproducibility was observed for the five independent precipitations, which resulted in 120/181 (66%) reproducible viral ligands (in≥2/5 experiments), while 61/181 (34%) were uniquely identified in individual experiments (FIG. 7). In total, analyses of infected MRC-5 fibroblasts allowed the identification of 198 unique HCMV-derived HLA ligands, of which 78, 15, 66, 31, 3, and 5 are restricted to HLA-A*02:01, -A*29:02, -B*07:02, -B*44:02, -C*05:01, and -C*07:02, respectively (FIG. 2d). Due to the applied 5% false discovery rate (FDR) in data processing, the identified 7/2, (0.34%) and 3/1, (0.27%) viral peptides in mock controls (FIG. 2a) are most likely false-positive annotations. In order to estimate the actual false discovery rate of HCMV-derived peptides, the inventors compared fragment spectra of 50 randomly selected A*02:01 and B*44:02-restricted synthetic peptides to their natural counterparts. Fragmentation patterns matched for 48/50 (96%) spectrum pairs by manual validation, which indicates an overall false-positive annotation rate of HCMV-derived HLA ligands of <5%. To extend the set of HCMV-derived ligands to additional HLA allotypes, the inventors next infected primary human foreskin fibroblasts (HF-99/7) with the ΔUS2-US11 deletion mutant. Peptide extracts from mock treated and infected cells (one sample each) were analyzed in three LC-MS/MS runs yielding a total number of 2,839 and 5,511 HLA ligands, respectively (FIG. 2a). Of these, 37, 44, 21, 43, 17, and 4 viral peptides (altogether 181) were restricted to HLA-A*01:01, -A*03:01, -B*08:01, -B*51:01, -C*01:02, and -C*07:01, respectively (FIG. 2d). The HLA annotation of 15 peptides was ambiguous. Therefore, in total, 368 unique viral HLA-I ligands were identified from two different fibroblast cell cultures. Eleven ligands were found on both cell lines. The inventors had speculated that an infection time of 48 hrs would allow the detection of peptides originating from proteins with various expression kinetics (36). Indeed, the source proteins of the identified ligands represent all classes of gene expression. IFNγ ELISpot screening validates numerous HCMV-derived ligands to be T-cell epitopes.

All viral ligands identified from MRC-5 cells and the top ranked ligands from HF-99/7 cells (≥70% SYFPEITHI score, ≤50 nM IC50 and/or <0.5% NetMHC percentile rank) were further tested for immunogenicity. All peptides were synthesized in house and tested for memory T-cell responses in at least seven different HCMV seropositive HLA-matched individuals by IFNγ ELISpot assay. HLA restriction and virus-specificity of these T-cell responses was confirmed using HLA mismatched and HCMV seronegative donors as controls. In total, 28% of all peptides were tested positive in at least one individual. This percentage was roughly the same across all HLA restrictions (FIG. 3a). Although the inventors performed the ligandome analysis at only one time point (48 h.p.i.), all temporal classes of gene expression (36) were present among the source proteins of the identified epitopes (FIG. 3b). As expected, normalized spot counts of IFNγ ELISpots after peptide stimulation were donor dependent and in part highly variable (FIGS. 3c, 8, and 9). Dependent on the frequency of recognition the inventors grouped the peptides in to three categories: negative (no memory response in any individual), subdominant (recognized by <50% of individuals) and dominant (recognized by ≥50% of individuals). Interestingly, in addition to the well-known epitopes derived from pp65, the inventors found a number of other highly immunogenic peptides for each HLA restriction. Most immunogenic peptides in proportion to the number of tested peptides were found for HLA-A*01:01, whereas the highest percentage of dominant epitopes was found for HLA-B*07:02 (FIG. 3a). The inventors observed that for peptides with higher recognition rates the mean number of specific memory T cells after 12 day stimulation is often higher compared to peptides with lower recognition rates (FIGS. 3c and 8). To exclude that this effect is caused by competitive effects among the different epitopes during the 12 day amplification, the inventors additionally performed ex vivo IFNγ ELISpots without this prestimulation. The dominant epitopes were retested with PBMC samples of previously tested positive donors. Only a few of the best epitopes elicited frequent, detectable responses ex vivo (Table 1). In most cases, memory T-cell numbers were too small to be detectable ex vivo but underwent, in part massive, amplification (up to 1000-fold) upon pre-stimulation (FIG. 3d). The amplification rate was highly individual for epitopes as well as for donors. In total, 103 HCMV-derived T-cell epitopes were identified, whereof 26 were shown to be dominant (Table 2). In case of positive results in HLA-I mismatched ELISpots, the respective peptide was tested for the next best predicted HLA-I allele in ELISpots and/or for CD8+/CD4+ T-cell responses by intracellular cytokine staining (ICS). Three peptides elicited responses by CD4+ T cells, indicating binding to HLA class II. Three epitopes (UL147A 2-10, UL34 180-188, and UL26 61-69) are potentially able to bind to more than one HLA-I allotype since they stimulated T cells of different donors harboring either of two well predicted alleles. In summary, in addition to seven previously described epitopes, the inventors were able to identify 96 novel HCMV-derived T-cell epitopes. As the inventors have observed a long time ago (20), ELISpot experiments revealed that HCMV-specific T-cell responses directed against a broad range of antigens exist within one donor; up to eight epitopes restricted by one specific HLA-I allotype were recognized in parallel (FIG. 9). While most of the donors showed responses to a similar set of epitopes, some donors had highly individual patterns of recognition.

### HCMV-Specific Memory T Cells are Multifunctional

Peptide and HLA specificity of memory T cells was tested by HLA tetramer staining after 12 day amplification in vitro (Table 1 and FIG. 4a). The inventors were able to show distinct HCMV-specific CD8+ T-cell populations for all but one (UL44 259-267) dominant epitopes in several PBMC samples (Table 2). Specific T-cell populations ranged from 0.3% to 52% for one specificity. Functional activity of memory T cells after stimulation with HCMV peptides could be demonstrated by ICS via detection of IFNγ and TNF (FIG. 4b, Table 1). Predicted HLA restriction could be confirmed for 26 of 27 dominant epitopes. Stimulation with UL44 259-267 resulted in a T-cell response mediated by CD4+ cells. Also, the inventors could demonstrate that some epitopes elicit T-cell responses restricted to more than one HLA-I allotype. UL46 76-84 was able to activate CD4+ and CD8+ T cells in different PBMC samples. T-cell responses to UL26 61-69 were detected in seven B*08+/B*51− and B*08-/B*51+ samples and were mediated by CD8+ T cells in all tested donors. Tetramer stainings demonstrated B*08 and B*51 restriction of the epitope. However, mismatch ELISpots with B*08-/B*51− PBMC samples also showed responses indicating a binding to yet more HLA allotypes which will have to be further investigated. In summary, the inventors were able to further characterize dominant HCMV-derived epitopes using ICS, tetramer staining and mismatch experiments. HCMV-specific CD8+ T-cell clones effectively kill peptide-loaded or infected target cells For examination of cytotoxic activity CD8+ T-cell clones specific for UL23 22-30 (B*07) were generated. Specificity and activity of the clones were assessed by HLA tetramer staining and ICS. T-cell clones used for cytotoxicity experiments were highly specific and showed secretion of IFNγ, TNF and the degranulation marker CD107a (FIG. 5a). For further cytotoxicity analysis the inventors applied the XCelligence system. Without reactive CD8+ T cells the HCMV-infected MRC-5 cells displayed a specific cell index pattern as the infection proceeded and changed the cell morphology. A few hours after infection MRC-5 cells started to round up and lose adherence in comparison to uninfected cells. This is detected by a lower cell index in the xCELLigence system. Around 20 h.p.i., cell indices increased again as MRC-5 cells started to re-adhere. Finally, the cell index dropped drastically 3-5 days post infection (depending on the applied MOI) due to cell lysis. For optimal measurement of T-cell dependent cytotoxicity, effector T-cells were added approximately 48 h.p.i. This allowed the infected cells to reach higher cell index values prior to late cell index drop due to infection. To test peptide specificity of the CD8+ T-cell clones, mock treated MRC-5 cells were loaded with specific or unspecific peptides or infected with the ΔUS2-6 mutant, and effector cells were added in a 5:1 effector to target cell (E:T) ratio. Killing of peptide loaded cells occurred very fast and was highly specific (FIG. 5b); within 12 h almost all UL23 22-30 loaded target cells were killed by the specific T-cell clone. Killing of ΔUS2-6-infected cells was delayed but equally efficient. Cell index values were much higher (less cell lysis) for cells loaded with an unspecific or no peptide when co-cultured with the UL23 22-30-specific T-cell clone. An E:T ratio dependent killing of ΔUS2-6 infected MRC-5 cells started a few hours after addition of effector cells (FIG. 5c), it reached 50% after 18 h (E:T of 1:1 and higher) and after 36 h (corresponding to 84 h.p.i.) almost all infected cells were killed by the peptide-specific CD8+ T-cell clones. Interestingly, despite the minimal expression of HLA-I/peptide complexes on the surface of AD169VarL infected cells, a cytolytic effect was observed at higher E:T ratios, when normalized to infected cells without effector cells (FIG. 5d). In accordance to the assumption that low a mounts of HLA-I/peptide complexes are responsible for the slow killing of AD169VarL infected cells, the additional loading of specific peptide led to a dramatic increase of lysis (FIG. 5d).

### Comparison of in Silico Epitope Prediction to Mass Spectrometric HLA-I Ligand Identification

To compare the inventors' approach of identifying epitopes with an established in silico prediction method, the inventors applied the prediction tools SYFPEITHI and NetMHCpan3.0 to the proteome of HCMV. The inventors ranked all peptides according to their prediction score and determined the position of the inventors' dominant epitopes 8 within this dataset (Table 2). For both SYFPEITHI and NetMHC, 25 of the 26 identified dominant epitopes are among the top-scoring 2% of all predicted peptides. This is in line with the previous experience with SYFPEITHI that the top 2% of predicted peptides usually contain the natural T-cell epitopes (37). NetMHC categorizes its predicted peptides into weak (affinity<500 nM, % rank<2) and strong binders (affinity<50 nM, % rank<0.5). Thus, it would be necessary to test approximately 1,300 (SYFPEITHI) or 2,000 (NetMHC) peptides per HLA-I allotype and length variant in order to screen epitopes from the entire HCMV proteome within these thresholds.

### REFERENCES AS CITED

### References

1. Staras S A, Dollard S C, Radford K W, Flanders W D, Pass R F, Cannon M J (2006) Seroprevalence of cytomegalovirus infection in the United States, 1988-1994. Clin Infect Dis 43:1143-1151.

2. Fulop T, Larbi A, Pawelec G (2013) Human T cell aging and the impact of persistent viral infections. Front Immunol 4:271.

3. Quinnan G V, Jr., Kirmani N, Rook A H, Manischewitz J F, Jackson L, Moreschi G, Santos G W, Saral R, Burns W H (1982) Cytotoxic t cells in cytomegalovirus infection: HLA-restricted T-lymphocyte and non-T-lymphocyte cytotoxic responses correlate with recovery from cytomegalovirus infection in bone-marrow-transplant recipients. The New England journal of medicine 307:7-13.

4. Reusser P, Riddell S R, Meyers J D, Greenberg P D (1991) Cytotoxic T-lymphocyte response to cytomegalovirus after human allogeneic bone marrow transplantation: pattern of recovery and correlation with cytomegalovirus infection and disease. Blood 78:1373-1380.

5. Einsele H, Roosnek E, Rufer N, Sinzger C, Riegler S, Loffler J, Grigoleit U, Moris A, Rammensee H G, Kanz L, Kleihauer A, Frank F, Jahn G, Hebart H (2002) Infusion of cytomegalovirus (CMV)-specific T cells for the treatment of CMV infection not responding to antiviral chemotherapy. Blood 99:3916-3922.

6. Zhou Y F, Leon M B, Waclawiw M A, Popma J J, Yu Z X, Finkel T, Epstein S E (1996) Association between prior cytomegalovirus infection and the risk of restenosis after coronary atherectomy. The New England journal of medicine 335:624-630.

7. Harkins L, Volk A L, Samanta M, Mikolaenko I, Britt W J, Bland K I, Cobbs C S (2002) Specific localisation of human cytomegalovirus nucleic acids and proteins in human colorectal cancer. Lancet 360:1557-1563.

8. Soderberg-Naucler C (2006) Does cytomegalovirus play a causative role in the development of various inflammatory diseases and cancer? J Intern Med 259:219-246.

9. Soderberg-Naucler C (2008) HCMV microinfections in inflammatory diseases and cancer. J Clin Virol 41:218-223.

10. Li S, Zhu J, Zhang W, Chen Y, Zhang K, Popescu L M, Ma X, Lau W B, Rong R, Yu X, Wang B, Li Y, Xiao C, Zhang M, Wang S, Yu L, Chen A F, Yang X, Cai J (2011) Signature microRNA expression profile of essential hypertension and its novel link to human cytomegalovirus infection. Circulation 124:175-184.

11. McLaughlin-Taylor E, Pande H, Forman S J, Tanamachi B, Li C R, Zaia J A, Greenberg P D, Riddell S R (1994) Identification of the major late human cytomegalovirus matrix protein pp65 as a target antigen for CD8+ virus-specific cytotoxic T lymphocytes. J Med Virol 43:103-110.

12. Wills M R, Carmichael A J, Mynard K, Jin X, Weekes M P, Plachter B, Sissons J G (1996) The human cytotoxic T-lymphocyte (CTL) response to cytomegalovirus is dominated by structural protein pp65: frequency, specificity, and T-cell receptor usage of pp65-specific CTL. J Virol 70:7569-7579.

13. Kern F, Surel I P, Faulhaber N, Frommel C, Schneider-Mergener J, Schonemann C, Reinke P, Volk H D (1999) Target structures of the CD8(+)-T-cell response to human cytomegalovirus: the 72-kilodalton major immediate-early protein revisited. J Virol 73:8179-8184.

14. Akiyama Y, Maruyama K, Mochizuki T, Sasaki K, Takaue Y, Yamaguchi K (2002) Identification of HLA-A24-restricted CTL epitope encoded by the matrix protein pp65 of human cytomegalovirus. Immunol Lett 83:21-30.

15. Le Roy E, Davignon J L (2005) Human cytomegalovirus-specific CD4(+) T-cell clones recognize cross-reactive peptides from the immediate early 1 protein. Viral Immunol 18:391-396.

16. Elkington R, Walker S, Crough T, Menzies M, Tellam J, Bharadwaj M, Khanna R (2003) Ex vivo profiling of CD8+-T-cell responses to human cytomegalovirus reveals broad and multispecific reactivities in healthy virus carriers. J Virol 77:5226-5240.

17. Manley T J, Luy L, Jones T, Boeckh M, Mutimer H, Riddell S R (2004) Immune evasion proteins of human cytomegalovirus do not prevent a diverse CD8+ cytotoxic T-cell response in natural infection. Blood 104:1075-1082.

18. Sylwester A W, Mitchell B L, Edgar J B, Taormina C, Pelte C, Ruchti F, Sleath P R, Grabstein K H, Hosken N A, Kern F, Nelson J A, Picker L J (2005) Broadly targeted human cytomegalovirus-specific CD4+ and CD8+ T cells dominate the memory compartments of exposed subjects. J Exp Med 202:673-685.

19. Hebart H, Daginik S, Stevanovic S, Grigoleit U, Dobler A, Baur M, Rauser G, Sinzger C, Jahn G, Loeffler J, Kanz L, Rammensee H G, Einsele H (2002) Sensitive detection of human cytomegalovirus peptide-specific cytotoxic T-lymphocyte responses by interferon-gamma-enzyme-linked immunospot assay and flow cytometry in healthy individuals and in patients after allogeneic stem cell transplantation. Blood 99:3830-3837.

20. Nastke M D, Herrgen L, Walter S, Wernet D, Rammensee H G, Stevanovic S (2005) Major contribution of codominant CD8 and CD4 T cell epitopes to the human cytomegalovirus-specific T cell repertoire. Cell Mol Life Sci 62:77-86.

21. McMurtrey C P, Lelic A, Piazza P, Chakrabarti A K, Yablonsky E J, Wahl A, Bardet W, Eckerd A, Cook R L, Hess R, Buchli R, Loeb M, Rinaldo C R, Bramson J, Hildebrand W H (2008) Epitope discovery in West Nile virus infection: Identification and immune recognition of viral epitopes. Proc Natl Acad Sci USA 105:2981-2986.

22. Meyer V S, Kastenmuller W, Gasteiger G, Franz-Wachtel M, Lamkemeyer T, Rammensee H G, Stevanovic S, Sigurdardottir D, Drexler I (2008) Long-term immunity against actual poxviral HLA ligands as identified by differential stable isotope labeling. J Immunol 181:6371-6383.

23. Gunther P S, Peper J K, Faist B, Kayser S, Hartl L, Feuchtinger T, Jahn G, Neuenhahn M, Busch D H, Stevanovic S, Dennehy K M (2015) Identification of a Novel Immunodominant HLA-B*07:02-restricted Adenoviral Peptide Epitope and Its Potential in Adoptive Transfer Immunotherapy. J Immunother 38:267-275.

24. Ternette N, Yang H, Partridge T, Llano A, Cedeno S, Fischer R, Charles P D, Dudek N L, Mothe B, Crespo M, Fischer W M, Korber B T, Nielsen M, Borrow P, Purcell A W, Brander C, Dorrell L, Kessler B M, Hanke T (2016) Defining the HLA class I-associated viral antigen repertoire from HIV-1-infected human cells. European journal of immunology 46:60-69.

25. Jones T R, Wiertz E J, Sun L, Fish K N, Nelson J A, Ploegh H L (1996) Human cytomegalovirus US3 impairs transport and maturation of major histocompatibility complex class I heavy chains. Proc Natl Acad Sci USA 93:11327-11333.

26. Ahn K, Gruhler A, Galocha B, Jones T R, Wiertz E J, Ploegh H L, Peterson P A, Yang Y, Fruh K (1997) The ER-luminal domain of the HCMV glycoprotein US6 inhibits peptide translocation by TAP. Immunity 6:613-621.

27. Gewurz B E, Gaudet R, Tortorella D, Wang E W, Ploegh H L, Wiley D C (2001) Antigen presentation subverted: Structure of the human cytomegalovirus protein US2 bound to the class I molecule HLA-A2. Proc Natl Acad Sci USA 98:6794-6799.

28. Furman M H, Dey N, Tortorella D, Ploegh H L (2002) The human cytomegalovirus US10 gene product delays trafficking of major histocompatibility complex class I molecules. J Virol 76:11753-11756.

29. Hegde N R, Tomazin R A, Wisner T W, Dunn C, Boname J M, Lewinsohn D M, Johnson D C (2002) Inhibition of HL-DR-assembly, transport, and loading by human cytomegalovirus glycoprotein US3: a novel mechanism for evading major histocompatibility complex class II antigen presentation. J Virol 76:10929-10941.

30. Odeberg J, Plachter B, Branden L, Soderberg-Naucler C (2003) Human cytomegalovirus protein pp65 mediates accumulation of HLA-DR in lysosomes and destruction of the HLA-DR alpha-chain. Blood 101:4870-4877.

31. Wiertz E J, Tortorella D, Bogyo M, Yu J, Mothes W, Jones T R, Rapoport T A, Ploegh H L (1996) Sec61-mediated transfer of a membrane protein from the endoplasmic reticulum to the proteasome for destruction. Nature 384:432-438.

32. Wiertz E J, Jones T R, Sun L, Bogyo M, Geuze H J, Ploegh H L (1996) The human cytomegalovirus US11 gene product dislocates MHC class I heavy chains from the endoplasmic reticulum to the cytosol. Cell 84:769-779.

33. Jones T R, Sun L (1997) Human cytomegalovirus US2 destabilizes major histocompatibility complex class I heavy chains. J Virol 71:2970-2979.

34. Hewitt E W, Gupta S S, Lehner P J (2001) The human cytomegalovirus gene product US6 inhibits ATP binding by TAP. EMBO J 20:387-396.

35. Le V T, Trilling M, Hengel H (2011) The cytomegaloviral protein pUL138 acts as potentiator of tumor necrosis factor (TNF) receptor 1 surface density to enhance ULb′-encoded modulation of TNF-alpha signaling. J Virol 85:13260-13270.

36. Weekes M P, Tomasec P, Huttlin E L, Fielding C A, Nusinow D, Stanton R J, Wang E C, Aicheler R, Murrell I, Wilkinson G W, Lehner P J, Gygi S P (2014) Quantitative temporal viromics: an approach to investigate host-pathogen interaction.

Cell 157:1460-1472.

37. Rammensee H, Bachmann J, Emmerich N P, Bachor O A, Stevanovic S (1999) SYFPEITHI: database for MHC ligands and peptide motifs. Immunogenetics 50:213-219.

38. Vescovini R, Biasini C, Fagnoni F F, Telera A R, Zanlari L, Pedrazzoni M, Bucci L, Monti D, Medici M C, Chezzi C, Franceschi C, Sansoni P (2007) Massive load of functional effector CD4+ and CD8+ T cells against cytomegalovirus in very old subjects. J Immunol 179:4283-4291.

39. Hengel H, Lucin P, Jonjic S, Ruppert T, Koszinowski U H (1994) Restoration of cytomegalovirus antigen presentation by gamma interferon combats viral escape. J Virol

40. Busche A, Jirmo A C, Welten S P, Zischke J, Noack J, Constabel H, Gatzke A K, Keyser K A, Arens R, Behrens G M, Messerle M (2013) Priming of CD8+ T cells against cytomegalovirus-encoded antigens is dominated by cross-presentation. J Immunol 190:2767-2777.

41. Hengel H, Reusch U, Geginat G, Holtappels R, Ruppert T, Hellebrand E, Koszinowski U H (2000) Macrophages escape inhibition of major histocompatibility complex class I-dependent antigen presentation by cytomegalovirus. J Virol 74:7861-7868.

42. Frascaroli G, Lecher C, Varani S, Setz C, van der Merwe J, Brune W, Mertens T (2018) Human Macrophages Escape Inhibition of Major Histocompatibility Complex-Dependent Antigen Presentation by Cytomegalovirus and Drive Proliferation and Activation of Memory CD4(+) and CD8(+) T Cells. Front Immunol 9:1129.

43. Rolland M, Nickle D C, Deng W, Frahm N, Brander C, Learn G H, Heckerman D, Jojic N, Jojic V, Walker B D, Mullins J I (2007) Recognition of HIV-1 peptides by host CTL is related to HIV-1 similarity to human proteins. PLoS One 2:e823.

44. Wolff M, Rutebemberwa A, Mosbruger T, Mao Q, Li H M, Netski D, Ray S C, Pardoll D, Sidney J, Sette A, Allen T, Kuntzen T, Kavanagh D G, Kuball J, Greenberg P D, Cox A L (2008) Hepatitis C virus immune escape via exploitation of a hole in the T cell repertoire. J Immunol 181:6435-6446.

45. Calis J J, de Boer R J, Kesmir C (2012) Degenerate T-cell recognition of peptides on MHC molecules creates large holes in the T-cell repertoire. PLoS computational biology 8:e1002412.

46. Stern-Ginossar N, Weisburd B, Michalski A, Le V T, Hein M Y, Huang S X, Ma M, Shen B, Qian S B, Hengel H, Mann M, Ingolia N T, Weissman J S (2012) Decoding human cytomegalovirus. Science (New York, N.Y.) 338:1088-1093.

47. Erhard F, Halenius A, Zimmermann C, L'Hernault A, Kowalewski D J, Weekes M P, Stevanovic S, Zimmer R, Dolken L (2018) Improved Ribo-seq enables identification of cryptic translation events. Nat Methods 15:363-366.

48. Hoist P J, Jensen B A, Ragonnaud E, Thomsen A R, Christensen J P (2015) Targeting of non-dominant antigens as a vaccine strategy to broaden T-cell responses during chronic viral infection. PLoS One 10.

49. Steffensen M A, Pedersen L H, Jahn M L, Nielsen K N, Christensen J P, Thomsen A R (2016) Vaccine Targeting of Subdominant CD8+ T Cell Epitopes Increases the Breadth of the T Cell Response upon Viral Challenge, but May Impair Immediate Virus Control. J Immunol 196:2666-2676.

50. Panagioti E, Klenerman P, Lee L N, van der Burg S H, Arens R (2018) Features of Effective T Cell-Inducing Vaccines against Chronic Viral Infections. Front Immunol 9.

51. Le V T K, Trilling M, Hengel H (2011) The Cytomegaloviral Protein pUL138 Acts as Potentiator of Tumor Necrosis Factor (TNF) Receptor 1 Surface Density To Enhance ULb′-Encoded Modulation of TNF-α Signaling. Journal of Virology 85:13260-13270.

52. Wagner M, Gutermann A, Podlech J, Reddehase M J, Koszinowski U H (2002) Major Histocompatibility Complex Class I Allele-specific Cooperative and Competitive Interactions between Immune Evasion Proteins of Cytomegalovirus. The Journal of Experimental Medicine 196:805-816.

53. Atalay R, Zimmermann A, Wagner M, Borst E, Benz C, Messerle M, Hengel H (2002) Identification and Expression of Human Cytomegalovirus Transcription Units Coding for Two Distinct Fcγ Receptor Homologs. Journal of Virology 76:8596-8608.

54. Kowalewski D J, Stevanović S (2013) Biochemical Large-Scale Identification of MHC Class I Ligands. Antigen Processing: Methods and Protocols, ed van Endert P (Humana Press, Totowa, N.J.), pp 145-157.

55. Kowalewski D J, Schuster H, Backert L, Berlin C, Kahn S, Kanz L, Salih H R, Rammensee H G, Stevanovic S, Stickel J S (2015) HLA ligandome analysis identifies the underlying specificities of spontaneous antileukemia immune responses in chronic lymphocytic leukemia (CLL). Proc Natl Acad Sci USA 112:29.

56. Eng J K, McCormack A L, Yates J R (1994) An approach to correlate tandem mass spectral data of peptides with amino acid sequences in a protein database. J Am Soc Mass Spectrom 5:976-989.

57. Kall L, Canterbury J D, Weston J, Noble W S, MacCoss M J (2007) Semi-supervised learning for peptide identification from shotgun proteomics datasets. Nat Methods 4:923-925.

58. Lundegaard C, Lund O, Nielsen M (2008) Accurate approximation method for prediction of class I MHC affinities for peptides of length 8, 10 and 11 using prediction tools trained on 9mers. Bioinformatics 24:1397-1398.

59. Nielsen M, Andreatta M (2016) NetMHCpan-3.0; improved prediction of binding to MHC class I molecules integrating information from multiple receptor and peptide length datasets. Genome medicine 8:33.

60. Garboczi D N, Hung D T, Wiley D C (1992) HLA-A2-peptide complexes: refolding and crystallization of molecules expressed in Escherichia coli and complexed with single antigenic peptides. Proc Natl Acad Sci USA 89:3429-3433.

61. Rodenko B, Toebes M, Hadrup S R, van Esch W J, Molenaar A M, Schumacher T N, Ovaa H (2006) Generation of peptide-MHC class I complexes through UV-mediated ligand exchange. Nat Protoc 1:1120-1132.

62. Peper J K, Bosmuller H C, Schuster H, Guckel B, Horzer H, Roehle K, Schafer R, Wagner P, Rammensee H G, Stevanovic S, Fend F, Staebler A (2016) HLA ligandomics identifies histone deacetylase 1 as target for ovarian cancer immunotherapy. Oncoimmunology 5:e1065369.

63. Britten C M, Gouttefangeas C, Welters M J, Pawelec G, Koch S, Ottensmeier C, Mander A, Walter S, Paschen A, Muller-Berghaus J, Haas I, Mackensen A, Kollgaard T, thor Straten P, Schmitt M, Giannopoulos K, Maier R, Veelken H, Bertinetti C, Konur A, Huber C, Stevanovic S, Wolfel T, van der Burg S H (2008) The CIMT-monitoring panel: a two-step approach to harmonize the enumeration of antigen-specific CD8+ T lymphocytes by structural and functional assays. Cancer Immunol Immunother 57:289-302.

64. Peper J K, Schuster H, Loffler M W, Schmid-Horch B, Rammensee H G, Stevanovic S (2014) An impedance-based cytotoxicity assay for real-time and label-free assessment of T-cell-mediated killing of adherent cells. J Immunol Methods 405:192-198.

1-22. (canceled)

23. A peptide comprising an amino acid sequence selected from the group consisting of SEQ ID NO: 1 to SEQ ID NO: 101, and variant sequences of SEQ ID NO: 1 to SEQ ID NO: 101 that comprise one amino acid exchange and bind to molecule(s) of the major histocompatibility complex (MHC) and/or induce T cells cross-reacting with said variant peptide, and pharmaceutical acceptable salts thereof, wherein said peptide has an overall length of between 8 and 30 amino acids.

24. The peptide or variant according to claim 23, wherein said peptide consists of an amino acid sequence according to any of SEQ ID NO: 1 to SEQ ID NO: 100 and optionally comprising an extension of one N- and/or one C-terminal amino acid.

25. The peptide or variant according to claim 23, wherein the amino acid sequence is selected from SEQ ID NO: 1 to 4, 24 to 29, 40, 41, 51 to 55, 67, 68, 80, 87 to 89, and 99 to 101.

26. The peptide or variant thereof according to claim 23, wherein said peptide is modified and/or includes non-peptide bonds.

27. The peptide or variant thereof according to claim 23, wherein said peptide is part of a fusion protein, comprising the N-terminal amino acids of the HLA-DR antigen-associated invariant chain (li).

28. A soluble or membrane-bound antibody, that specifically binds to the peptide or variant thereof according to claim 23, and/or the peptide or variant thereof when bound to an MHC molecule.

29. A recombinant, soluble or membrane-bound T cell receptor that is reactive with an HLA ligand, wherein said ligand is at least 75% identical to an amino acid sequence according to claim 24.

30. The T cell receptor according to claim 29, wherein said T cell receptor is a soluble molecule, and optionally comprises an effector function.

31. A nucleic acid encoding:
a peptide or variant thereof according to claim 23;
a soluble or membrane-bound antibody that specifically binds to the peptide or variant thereof according to claim 23 and/or a peptide or variant thereof when bound to an MHC molecule; or
a recombinant, soluble or membrane-bound T cell receptor that is reactive with an HLA ligand, wherein said ligand is at least 75% identical to an amino acid sequence according to any of SEQ ID NO: 1 to SEQ ID NO: 100, and optionally comprising an extension of one N- and/or one C-terminal amino acid;
wherein said nucleic acid is optionally linked to a heterologous promoter sequence.

32. An expression vector expressing the nucleic acid according to claim 31.

33. A recombinant host cell comprising:
a recombinant peptide according to claim 23;
a soluble or membrane-bound antibody that specifically binds to the peptide or variant thereof according to claim 23, and/or a peptide or variant thereof when bound to an MHC molecule;
a recombinant, soluble or membrane-bound T cell receptor that is reactive with an HLA ligand, wherein said ligand is at least 75% identical to an amino acid sequence according to any of SEQ ID NO:1 to SEQ ID NO:100 and optionally comprising an extension of one N- and/or one C-terminal amino acid;
a nucleic acid encoding a peptide or variant thereof according to claim 23; a soluble or membrane-bound antibody that specifically binds to the peptide or variant thereof according to claim 23; a recombinant, soluble or membrane-bound T cell receptor that is reactive with an HLA ligand, wherein said ligand is at least 75% identical to an amino acid sequence according to any of SEQ ID NO: 1 to SEQ ID NO: 100 and optionally comprising an extension of one N- and/or one C-terminal amino acid; wherein said nucleic acid is optionally linked to a heterologous promoter sequence.

34. A method for producing:
a peptide comprising an amino acid sequence selected from the group consisting of SEQ ID NO: 1 to SEQ ID NO: 101, and variant sequences of SEQ ID NO: 1 to SEQ ID NO: 101 that comprise one amino acid exchange and bind to molecule(s) of the major histocompatibility complex (MHC) and/or induce T cells cross-reacting with said variant peptide, wherein said peptide has an overall length of between 8 and 30 amino acids;
a soluble or membrane-bound antibody, that specifically binds to said peptide or variant thereof, and/or said peptide or variant thereof when bound to an MHC molecule;
or a recombinant, soluble or membrane-bound T cell receptor that is reactive with an HLA ligand, wherein said ligand is at least 75% identical to an amino acid sequence according to any of SEQ ID NO:1 to SEQ ID NO:100 and optionally comprising an extension of one N- and/or one C-terminal amino acid;
the method comprising culturing the host cell according to claim 33 that presents said peptide; or expresses said nucleic acid;
and isolating said peptide or variant thereof, said antibody, or said T cell receptor from said host cell and/or its culture medium.

35. An in vitro method for producing activated T lymphocytes, the method comprising contacting in vitro T cells with antigen loaded human class I or II MHC molecules expressed on the surface of a suitable antigen-presenting cell or an artificial construct mimicking an antigen-presenting cell for a period of time sufficient to activate said T cells in an antigen specific manner, wherein said antigen is a peptide according to claim 23.

36. An activated T lymphocyte, produced by the method according to claim 35, that selectively recognizes a cell that presents a peptide comprising an amino acid sequence selected from the group consisting of SEQ ID NO: 1 to SEQ ID NO: 101, and variant sequences of SEQ ID NO: 1 to SEQ ID NO: 101 that comprise one amino acid exchange and bind to molecule(s) of the major histocompatibility complex (MHC) and/or induce T cells cross-reacting with said variant peptide, wherein said peptide has an overall length of between 8 and 30 amino acids.

37. A pharmaceutical composition comprising at least one active ingredient selected from the group consisting of:
a peptide comprising an amino acid sequence selected from the group consisting of SEQ ID NO: 1 to SEQ ID NO: 101, and variant sequences of SEQ ID NO: 1 to SEQ ID NO:
101 that comprise one amino acid exchange and bind to molecule(s) of the major histocompatibility complex (MHC) and/or induce T cells cross-reacting with said variant peptide, and pharmaceutical acceptable salts thereof, wherein said peptide has an overall length of between 8 and 30 amino acids;
a soluble or membrane-bound antibody, that specifically binds to said peptide or variant thereof, and/or said peptide or variant thereof when bound to an MHC molecule;
a recombinant, soluble or membrane-bound T cell receptor that is reactive with an HLA ligand, wherein said ligand is at least 75% identical to an amino acid sequence according to any of SEQ ID NO:1 to SEQ ID NO:100 and optionally comprising an extension of one N- and/or one C-terminal amino acid;
a nucleic acid encoding said peptide or variant; said soluble or membrane-bound antibody; or said recombinant, soluble or membrane-bound T cell receptor; wherein said nucleic acid is optionally linked to a heterologous promoter sequence;
a recombinant host cell according to claim 33; or
an activated T lymphocyte that selectively recognizes a cell that presents a peptide comprising an amino acid sequence selected from the group consisting of SEQ ID NO: 1 to SEQ ID NO: 101, and variant sequences of SEQ ID NO: 1 to SEQ ID NO: 101 that comprise one amino acid exchange and bind to molecule(s) of the major histocompatibility complex (MHC) and/or induce T cells cross-reacting with said variant peptide, wherein said peptide has an overall length of between 8 and 30 amino acids; and
a pharmaceutically acceptable carrier, and optionally additional pharmaceutically acceptable excipients and/or stabilizers.

38. A method for producing a personalized anti-viral vaccine, said method comprising:
a) identifying at least one HCMV-associated peptide according to any one of SEQ ID NO: 1 to SEQ ID NO: 101 in a sample from said individual patient;
b) selecting at least one peptide as identified in said sample from step a), and
c) formulating the at least one peptide as selected in step b) into a personalized anti-viral vaccine.

39. A kit comprising:
a) a container comprising
a peptide comprising an amino acid sequence selected from the group consisting of SEQ ID NO: 1 to SEQ ID NO: 101, and variant sequences of SEQ ID NO: 1 to SEQ ID NO: 101 that comprise one amino acid exchange and bind to molecule(s) of the major histocompatibility complex (MHC) and/or induce T cells cross-reacting with said variant peptide, and pharmaceutical acceptable salts thereof, wherein said peptide has an overall length of between 8 and 30 amino acids;
a soluble or membrane-bound antibody, that specifically binds to said peptide or variant thereof, and/or said peptide or variant thereof when bound to an MHC molecule;
a recombinant, soluble or membrane-bound T cell receptor that is reactive with an HLA ligand, wherein said ligand is at least 75% identical to an amino acid sequence according to any of SEQ ID NO:1 to SEQ ID NO:100 and optionally comprising an extension of one N- and/or one C-terminal amino acid;
a nucleic acid encoding said peptide or variant; said soluble or membrane-bound antibody; or said recombinant, soluble or membrane-bound T cell receptor; wherein said nucleic acid is optionally linked to a heterologous promoter sequence;
a host cell according to claim 33; or
an activated T lymphocyte that selectively recognizes a cell that presents a peptide comprising an amino acid sequence selected from the group consisting of SEQ ID NO: 1 to SEQ ID NO: 101, and variant sequences of SEQ ID NO: 1 to SEQ ID NO: 101 that comprise one amino acid exchange and bind to molecule(s) of the major histocompatibility complex (MHC) and/or induce T cells cross-reacting with said variant peptide, wherein said peptide has an overall length of between 8 and 30 amino acids; or
a vaccine as produced a method for producing a personalized anti-viral vaccine, said method comprising:
a) identifying at least one HCMV-associated peptide according to any one of SEQ ID NO: 1 to SEQ ID NO: 101 in a sample from said individual patient;
b) selecting at least one peptide as identified in said sample from step a), and
c) formulating the at least one peptide as selected in step b) into a personalized anti-viral vaccine;
in solution or in lyophilized form;
b) optionally, a second container containing a diluent or reconstituting solution for the lyophilized formulation;
c) optionally, at least one additional peptide selected from the group consisting of SEQ ID NO: 1 to SEQ ID NO: 101,
d) optionally, instructions for (i) use of the solution or (ii) reconstitution and/or use of the lyophilized formulation, and
e) a substance or combination of substances acting as an adjuvant.

40. The kit according to claim 39, further comprising one or more of (iii) a buffer, (iv) a diluent, (v) a filter, (vi) a needle, (v) a syringe, and vi) a mixing device.

41. A method for treating HCMV infection in target cells in a patient, wherein said target cells present at least one peptide comprising an amino acid sequence according to any of SEQ ID NO: 1 to SEQ ID NO: 100 and optionally comprising an extension of one N-and/or one C-terminal amino acid, wherein said method comprises administering to said patient an effective amount of:
an activated T lymphocyte that selectively recognizes a cell that presents a peptide comprising an amino acid sequence selected from the group consisting of SEQ ID NO: 1 to SEQ ID NO: 101, and variant sequences of SEQ ID NO: 1 to SEQ ID NO: 101 that comprise one amino acid exchange and bind to molecule(s) of the major histocompatibility complex (MHC) and/or induce T cells cross-reacting with said variant peptide, wherein said peptide has an overall length of between 8 and 30 amino acids;
the pharmaceutical composition according to claim 37, and/or
the vaccine as produced by a method for producing a personalized anti-viral vaccine, said method comprising:
a) identifying at least one HCMV-associated peptide according to any one of SEQ ID NO: 1 to SEQ ID NO: 101 in a sample from said individual patient;
b) selecting at least one peptide as identified in said sample from step a), and
c) formulating the at least one peptide as selected in step b) into a personalized anti-viral vaccine.

42. The method according to claim 41, wherein said HCMV infection exhibits a co-morbidity with cancer, inflammatory diseases, hypertensive diseases, and/or pulmonary diseases.


SEQUENCE LISTING (116 sequences)

SEQ ID NO: 1 - protein, 9 aa, Human cytomegalovirus
GVLDAVWRV

SEQ ID NO: 2 - protein, 10 aa, Human cytomegalovirus
ALWDVALLEV

SEQ ID NO: 3 - protein, 9 aa, Human cytomegalovirus
TLIVNLVEV

SEQ ID NO: 4 - protein, 9 aa, Human cytomegalovirus
GLFAVENFL

SEQ ID NO: 5 - protein, 9 aa, Human cytomegalovirus
FLDENFKQL

SEQ ID NO: 6 - protein, 9 aa, Human cytomegalovirus
RLFDLPVYC

SEQ ID NO: 7 - protein, 9 aa, Human cytomegalovirus
RLQPNVPLV

SEQ ID NO: 8 - protein, 11 aa, Human cytomegalovirus
GLLAHIPALGV

SEQ ID NO: 9 - protein, 9 aa, Human cytomegalovirus
ALSPSTSKV

SEQ ID NO: 10 - protein, 9 aa, Human cytomegalovirus
SLYEANPEL

SEQ ID NO: 11 - protein, 9 aa, Human cytomegalovirus
TILDKILNV

SEQ ID NO: 12 - protein, 9 aa, Human cytomegalovirus
TLINGVWVV

SEQ ID NO: 13 - protein, 9 aa, Human cytomegalovirus
GLLAHIPAL

SEQ ID NO: 14 - protein, 10 aa, Human cytomegalovirus
ALYPEYIYTV

SEQ ID NO: 15 - protein, 9 aa, Human cytomegalovirus
GLNERLLSV

SEQ ID NO: 16 - protein, 10 aa, Human cytomegalovirus
ALFNQLVFTA

SEQ ID NO: 17 - protein, 9 aa, Human cytomegalovirus
FTDNVRFSV

SEQ ID NO: 18 - protein, 9 aa, Human cytomegalovirus
GLDQYLESV

SEQ ID NO: 19 - protein, 9 aa, Human cytomegalovirus
ALLGRLYFI

SEQ ID NO: 20 - protein, 9 aa, Human cytomegalovirus
NYNEQHYRY

SEQ ID NO: 21 - protein, 9 aa, Human cytomegalovirus
LYVGQFLAY

SEQ ID NO: 22 - protein, 10 aa, Human cytomegalovirus
SFFPKLQGNY

SEQ ID NO: 23 - protein, 9 aa, Human cytomegalovirus
FFPKLQGNY

SEQ ID NO: 24 - protein, 9 aa, Human cytomegalovirus
YPRPPGSGL

SEQ ID NO: 25 - protein, 10 aa, Human cytomegalovirus
TPRVTGGGAM

SEQ ID NO: 26 - protein, 9 aa, Human cytomegalovirus
SPRDAWIVL

SEQ ID NO: 27 - protein, 10 aa, Human cytomegalovirus
SPSRDRFVQL

SEQ ID NO: 28 - protein, 9 aa, Human cytomegalovirus
RPWKPGQRV

SEQ ID NO: 29 - protein, 9 aa, Human cytomegalovirus
SPRHLYISL

SEQ ID NO: 30 - protein, 11 aa, Human cytomegalovirus
IPMTFVDRDSL

SEQ ID NO: 31 - protein, 9 aa, Human cytomegalovirus
RPFPSTHQL

SEQ ID NO: 32 - protein, 9 aa, Human cytomegalovirus
RVSQPSLIL

SEQ ID NO: 33 - protein, 10 aa, Human cytomegalovirus
SPHPPTSVFL

SEQ ID NO: 34 - protein, 9 aa, Human cytomegalovirus
IPDYRSVSL

SEQ ID NO: 35 - protein, 8 aa, Human cytomegalovirus
APFGRVSV

SEQ ID NO: 36 - protein, 8 aa, Human cytomegalovirus
IPVERQAL

SEQ ID NO: 37 - protein, 9 aa, Human cytomegalovirus
APNYRQVEL

SEQ ID NO: 38 - protein, 9 aa, Human cytomegalovirus
LPNDHHYAL

SEQ ID NO: 39 - protein, 8 aa, Human cytomegalovirus
APYLRDTL

SEQ ID NO: 40 - protein, 10 aa, Human cytomegalovirus
SENGNLQVTY

SEQ ID NO: 41 - protein, 9 aa, Human cytomegalovirus
HETGVYQMW

SEQ ID NO: 42 - protein, 9 aa, Human cytomegalovirus
DEQVSKRSW

SEQ ID NO: 43 - protein, 9 aa, Human cytomegalovirus
SESEFIVRY

SEQ ID NO: 44 - protein, 9 aa, Human cytomegalovirus
EEQDYRALL

SEQ ID NO: 45 - protein, 9 aa, Human cytomegalovirus
RENAGVALY

SEQ ID NO: 46 - protein, 10 aa, Human cytomegalovirus
AEPNFPKNVW

SEQ ID NO: 47 - protein, 10 aa, Human cytomegalovirus
EEATALGREL

SEQ ID NO: 48 - protein, 9 aa, Human cytomegalovirus
SESLVAKRY

SEQ ID NO: 49 - protein, 9 aa, Human cytomegalovirus
LENGVTHRF

SEQ ID NO: 50 - protein, 10 aa, Human cytomegalovirus
REQAAIPQIY

SEQ ID NO: 51 - protein, 9 aa, Human cytomegalovirus
YADPFFLKY

SEQ ID NO: 52 - protein, 9 aa, Human cytomegalovirus
VTEHDTLLY

SEQ ID NO: 53 - protein, 10 aa, Human cytomegalovirus
RTDPATLTAY

SEQ ID NO: 54 - protein, 9 aa, Human cytomegalovirus
ITEIALDRY

SEQ ID NO: 55 - protein, 10 aa, Human cytomegalovirus
NTDFRVLELY

SEQ ID NO: 56 - protein, 10 aa, Human cytomegalovirus
FVEGPGFMRY

SEQ ID NO: 57 - protein, 9 aa, Human cytomegalovirus
SLDRFIVQY

SEQ ID NO: 58 - protein, 10 aa, Human cytomegalovirus
YTSRGALYLY

SEQ ID NO: 59 - protein, 9 aa, Human cytomegalovirus
TSETHFGNY

SEQ ID NO: 60 - protein, 10 aa, Human cytomegalovirus
GSDALPAGLY

SEQ ID NO: 61 - protein, 11 aa, Human cytomegalovirus
VTDYGNVAFKY

SEQ ID NO: 62 - protein, 11 aa, Human cytomegalovirus
LLDELGAVFGY

SEQ ID NO: 63 - protein, 11 aa, Human cytomegalovirus
ISENGNLQVTY

SEQ ID NO: 64 - protein, 9 aa, Human cytomegalovirus
VTDPEHLMM

SEQ ID NO: 65 - protein, 10 aa, Human cytomegalovirus
DLDFGDLLKY

SEQ ID NO: 66 - protein, 11 aa, Human cytomegalovirus
YSDRRDHVWSY

SEQ ID NO: 67 - protein, 9 aa, Human cytomegalovirus
GLYTQPRWK

SEQ ID NO: 68 - protein, 9 aa, Human cytomegalovirus
RVKNRPIYR

SEQ ID NO: 69 - protein, 10 aa, Human cytomegalovirus
RSALGPFVGK

SEQ ID NO: 70 - protein, 9 aa, Human cytomegalovirus
KLGGALQAK

SEQ ID NO: 71 - protein, 9 aa, Human cytomegalovirus
KLGYRPHAK

SEQ ID NO: 72 - protein, 10 aa, Human cytomegalovirus
RVYAYDTREK

SEQ ID NO: 73 - protein, 9 aa, Human cytomegalovirus
GVSSVTLLK

SEQ ID NO: 74 - protein, 9 aa, Human cytomegalovirus
RVDPNLRNR

SEQ ID NO: 75 - protein, 9 aa, Human cytomegalovirus
SVRLPYMYK

SEQ ID NO: 76 - protein, 9 aa, Human cytomegalovirus
RTFAGTLSR

SEQ ID NO: 77 - protein, 9 aa, Human cytomegalovirus
RLADVLIKR

SEQ ID NO: 78 - protein, 10 aa, Human cytomegalovirus
RSVRLPYMYK

SEQ ID NO: 79 - protein, 9 aa, Human cytomegalovirus
SVSSAPLNK

SEQ ID NO: 80 - protein, 9 aa, Human cytomegalovirus
YLVRRPMTI

SEQ ID NO: 81 - protein, 9 aa, Human cytomegalovirus
VMKFKETSF

SEQ ID NO: 82 - protein, 9 aa, Human cytomegalovirus
TPLLKRLPL

SEQ ID NO: 83 - protein, 9 aa, Human cytomegalovirus
HLKLRPATF

SEQ ID NO: 84 - protein, 8 aa, Human cytomegalovirus
FISSKHTL

SEQ ID NO: 85 - protein, 9 aa, Human cytomegalovirus
QLRSVIRAL

SEQ ID NO: 86 - protein, 8 aa, Human cytomegalovirus
MLRLLFTL

SEQ ID NO: 87 - protein, 8 aa, Human cytomegalovirus
LPLKMLNI

SEQ ID NO: 88 - protein, 9 aa, Human cytomegalovirus
FPVEVRSHV

SEQ ID NO: 89 - protein, 9 aa, Human cytomegalovirus
DARSRIHNV

SEQ ID NO: 90 - protein, 9 aa, Human cytomegalovirus
IPPPQIPFV

SEQ ID NO: 91 - protein, 9 aa, Human cytomegalovirus
IAIPHFMVV

SEQ ID NO: 92 - protein, 9 aa, Human cytomegalovirus
IPHNWFLQV

SEQ ID NO: 93 - protein, 9 aa, Human cytomegalovirus
VPAAVYTTV

SEQ ID NO: 94 - protein, 9 aa, Human cytomegalovirus
FPAHDWPEV

SEQ ID NO: 95 - protein, 9 aa, Human cytomegalovirus
MPVTHPPEV

SEQ ID NO: 96 - protein, 10 aa, Human cytomegalovirus
FPDATVPATV

SEQ ID NO: 97 - protein, 10 aa, Human cytomegalovirus
LPYLSAERTV

SEQ ID NO: 98 - protein, 9 aa, Human cytomegalovirus
SLFYRAVAL

SEQ ID NO: 99 - protein, 9 aa, Human cytomegalovirus
LPYPRGYTL

SEQ ID NO: 100 - protein, 9 aa, Human cytomegalovirus
LPHERHREL

SEQ ID NO: 101 - protein, 9 aa, Human cytomegalovirus
NLVPMVATV

SEQ ID NO: 102 - protein, 11 aa, Human cytomegalovirus
RPHERNGFTVL

SEQ ID NO: 103 - protein, 9 aa, Human immunodeficiency virus
ELRRKMMYM

SEQ ID NO: 104 - protein, 9 aa, Human cytomegalovirus
YLLPAIVHI

SEQ ID NO: 105 - protein, 9 aa, Human cytomegalovirus
RLRPGGKKK

SEQ ID NO: 106 - protein, 8 aa, Human cytomegalovirus
GGKKKYKL

SEQ ID NO: 107 - protein, 11 aa, Human cytomegalovirus
EEIPASDDVLF

SEQ ID NO: 108 - protein, 9 aa, Human cytomegalovirus
DPYKATSAV

SEQ ID NO: 109 - protein, 11 aa, Human cytomegalovirus
YSEHPTFTSQY

SEQ ID NO: 110 - protein, 10 aa, Human cytomegalovirus
SEHPTFTSQY

SEQ ID NO: 111 - protein, 11 aa, Human cytomegalovirus
QEFFWDANDIY

SEQ ID NO: 112 - protein, 9 aa, Human cytomegalovirus
VLEETSVML

SEQ ID NO: 113 - protein, 10 aa, Human cytomegalovirus
TPGPGVRYPL

SEQ ID NO: 114 - protein, 9 aa, Human cytomegalovirus
GSEELRSLY

SEQ ID NO: 115 - DNA, 82 bp, Human cytomegalovirus
caaaaagtctggtgagtcgtttccgagcgactcgagatgcactccgcttcagtctatataccagtgaattcgagctcggtac

SEQ ID NO: 116 - DNA, 82 bp, Human cytomegalovirus
taagacagccttacagcttttgagtctagacagggtaacagccttcccttgtaagacagagaccatgattacgccaagctcc